(19) United States
(12) Patent Application Publication
(10) Pub. No.: US 2026/0272973 A1
(43) Pub. Date: Sep. 17, 2026

(54) COMPOSITIONS AND METHODS FOR IMPROVED GENE SILENCING

(71) Applicant: Atalanta Therapeutics, Inc., Boston, MA (US)

(72) Inventors: Matthew HASSLER, Boston, MA (US); Garth A. KINBERGER, Hyde Park, MA (US)

(21) Appl. No.: 19/167,510

(22) PCT Filed: Mar. 22, 2024
(86) PCT No.: PCT/US2024/021207

§ 371 Date: Sep. 22, 2025

Related U.S. Application Data
(60) Provisional application No. 63/454,097, filed on Mar. 23, 2023.

(51) Int. Cl. A61K 31/713; A61K 31/712; A61K 31/7125; C12N 15/113

(52) U.S. Cl. CPC A61K 31/713; A61K 31/712; A61K 31/7125; C12N 15/113; C12N 2310/14; C12N 2310/315; C12N 2310/321; C12N 2310/322; C12N 2310/343; C12N 2310/3515

(57) ABSTRACT

The present disclosure provides single- or double-stranded interfering RNA molecules (e.g., siRNA) that exhibit improved gene silencing. The disclosure provides siRNA molecules having a fixed nucleobase region. For example, the antisense strand of the siRNA molecule may have a nucleobase region that contains one or more mismatches relative to the target mRNA molecule. Alternatively, or in addition, the antisense strand may contain a region of nucleobases having a sequence that is independent of the target mRNA. The fixed region may be contained within an overhang region of the antisense strand. The siRNA molecules may contain specific patterns of nucleoside modifications and internucleoside linkage modifications, as pharmaceutical compositions including the same. The siRNA molecules may be branched siRNA molecules, such as di-branched, tri-branched, or tetra-branched siRNA molecules. The disclosed siRNA molecules may further feature a 5′ phosphorus stabilizing moiety and/or a hydrophobic moiety. Additionally, the disclosure provides methods for delivering the siRNA molecule of the disclosure to the central nervous system of a subject, such as a subject identified as having a neurodegenerative disease.

### TECHNICAL FIELD

This disclosure relates to small interfering RNA (siRNA) molecules, and compositions containing the same, that exhibit improved gene silencing. The disclosure further describes methods for silencing of a target gene and the treatment of diseases that may benefit from gene silencing by delivering the siRNA molecules to a target tissue of a subject in need.

### BACKGROUND

In many species, introduction of double-stranded RNA (dsRNA) induces potent and specific gene silencing by way of RNA interference (RNAi). This phenomenon occurs in both plants and animals and has roles in viral defense and transposon silencing mechanisms. Short interfering RNAs (siRNAs), which are generally much shorter than the target gene, have been shown to be effective at gene silencing and are, therefore, useful as therapeutic agents for silencing genes to restore genetic and biochemical pathway activity from a disease state towards a normal, healthy state. However, there remains a need for siRNA constructs that exhibit improved gene silencing.

### SUMMARY OF THE INVENTION

The present disclosure provides compositions and methods for improved gene silencing. Accordingly, the disclosure provides siRNA molecules having a fixed nucleobase region. For example, the antisense strand of the siRNA molecule may have a nucleobase region that contains one or more mismatches relative to the target mRNA molecule. Alternatively, or in addition, the antisense strand may contain a region of nucleobases having a sequence that is independent of the target mRNA. The fixed region may be contained within an overhang region of the antisense strand.

The siRNA molecules of the disclosure can be delivered directly to a subject in need of gene silencing by way of, for example, injection intrathecally, intracerebroventricularly, intrastriatally, intraparenchymally, intra-cisterna magna injection, such as by catheterization, intravenous injection, subcutaneous injection, or intramuscular injection.

In an aspect, the disclosure provides a small interfering RNA (siRNA) molecule that contains an antisense strand and a sense strand having complementarity to a portion of the antisense strand; wherein:
(i) the antisense strand includes, in the 5′-to-3′ direction, a first region of linked nucleotides and a second region of linked nucleotides;
(ii) the first region has complementarity sufficient to hybridize to a portion of a target mRNA transcript;
(iii) the second region includes an overhang that extends beyond the sense strand; and
(iv) the second region has one or more nucleotide mismatches relative to the target mRNA transcript.

In some embodiments, the second region has from 1 to 4 (e.g., 1, 2, 3, or 4) nucleotide mismatches relative to the target mRNA transcript. In some embodiments, the second region has 1 nucleotide mismatch relative to the target mRNA transcript. In some embodiments, the second region has 2 nucleotide mismatches relative to the target mRNA transcript. In some embodiments, the second region has 3 nucleotide mismatches relative to the target mRNA transcript. In some embodiments, the second region has 4 nucleotide mismatches relative to the target mRNA transcript. In some embodiments, the second region has from 1 to 3 nucleotide mismatches relative to the target mRNA transcript. In some embodiments, the second region has from 2 to 4 nucleotide mismatches relative to the target mRNA transcript. In some embodiments, the second region has 3 or 4 nucleotide mismatches relative to the target mRNA transcript. In some embodiments, the second region has 1 or 2 nucleotide mismatches relative to the target mRNA transcript. In some embodiments, the second region has 2 or 3 nucleotide mismatches relative to the target mRNA transcript.

In another aspect, the disclosure provides an siRNA molecule containing an antisense strand and a sense strand having complementarity to a portion of the antisense strand; wherein:
(i) the antisense strand includes, in the 5′-to-3′ direction, a first region of linked nucleotides and a second region of linked nucleotides;
(ii) the first region has complementarity sufficient to hybridize to a portion of a target mRNA transcript;
(iii) the second region includes an overhang that extends beyond the sense strand; and
(iv) the second region has a nucleobase sequence that is independent of the nucleobase sequence of the target mRNA transcript.

In some embodiments of either of the aspects described above, the linked nucleotides are contiguous nucleotides.

In some embodiments of the siRNA molecules described herein, the nucleobase sequence of the second region imparts a formed mRNA cleavage product with an increased off-rate from a RISC complex following cleavage of the target mRNA relative to a corresponding antisense strand that is fully complementary to the target mRNA. In some embodiments, the nucleobase sequence of the second region imparts the antisense strand with an increased binding affinity for an endogenous Argonaute (AGO) protein relative to a corresponding antisense strand that is fully complementary to the target mRNA. In some embodiments, the nucleobase sequence of the second region improves the half-life of an endogenous complex containing the antisense strand and an AGO protein relative to a corresponding endogenous complex containing a corresponding antisense strand that is fully complementary to the target mRNA.

In some embodiments, the second region is from 1 to 10 (e.g., from 1 to 9, from 1 to 8, from 1 to 7, from 1 to 6, from 1 to 5, from 1 to 4, from 1 to 3, from 1 to 2, from 2 to 10, from 3 to 10, from 4 to 10, from 5 to 10, from 6 to 10, from 7 to 10, from 8 to 10, from 9 to 10, from 2 to 7, from 3 to 7, from 4 to 7, from 5 to 7, from 6 to 7, from 2 to 6, from 3 to 6, from 4 to 6, from 5 to 6, from 2 to 5, from 3 to 5, from 4 to 5, 1, 2, 3, 4, 5, 6, 7, 8, 9, or 10) nucleotides in length. In some embodiments, the second region is 1 nucleotide in length. In some embodiments, the second region is 2 nucleotides in length. In some embodiments, the second region is 3 nucleotides in length. In some embodiments, the second region is 4 nucleotides in length. In some embodiments, the second region is 5 nucleotides in length. In some embodiments, the second region is 6 nucleotides in length. In some embodiments, the second region is from 1 to 5 nucleotides in length. In some embodiments, the second region is from 1 to 4 nucleotides in length. In some embodiments, the second region is from 1 to 3 nucleotides in length. In some embodiments, the second region is from 2 to 4 nucleotides in length. In some embodiments, the second region is 3 or 4 nucleotides in length. In some embodiments, the second region is 1 or 2 nucleotides in length. In some embodiments, the second region is 2 or 3 nucleotides in length.

In some embodiments, the second region contains at least one uridine nucleotide. In some embodiments, the second region contains two uridine nucleotides.

In some embodiments, the second region contains at least one modified internucleoside linkage. In some embodiments of any of the fixed regions described herein, the fixed region includes at least one (e.g., 1, 2, 3, 4, 5, or more) modified internucleoside linkage.

In some embodiments, at least one modified intersubunit linkage is of Formula E1:

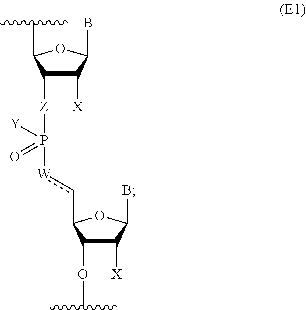

wherein:
each B is, independently, a base pairing moiety;
W is selected from the group consisting of O, S, B, BR², N, NR², OCH₂, OCH, CH₂, and CH, optionally wherein W is selected from the group consisting of OCH₂ and OCH;
each X is, independently, selected from the group consisting of halo (e.g., fluoro or chloro), hydroxy, and C₁₋₆ alkoxy, optionally wherein each X is, independently, selected from the group consisting of halo (e.g., fluoro or chloro) and C₁₋₆ alkoxy (e.g., methoxy, ethoxy, 2-methoxyethoxy, n-propoxy, sec-propoxy, n-butoxy, sec-butoxy, tert-butoxy, n-pentoxy, or n-heptoxy);
Y is selected from the group consisting of O⁻, OH, OR, NH⁻, NH₂, S⁻, and SH, optionally wherein Y is selected from the group consisting of O⁻, OH, and OR.
Z is selected from the group consisting of O, S, BR², NR², and CH₂;
R is a protecting group;
each R² is, independently, H or optionally substituted C₁-C₆ alkyl; and
[symbol] is an optional double bond.

In some embodiments of Formula E1, W is OCH₂.

In some embodiments of Formula E1, W is OCH and [symbol] is a double bond.

In some embodiments of Formula E1, Z is O.

In some embodiments of Formula E1, Z is CH₂.

In some embodiments of Formula E1, when Y is O⁻, either Z or W is not O.

In some embodiments of Formula E1, Z is CH₂ and W is CH₂. In some embodiments, the modified intersubunit linkage of Formula E1 is a modified intersubunit linkage of Formula E2:

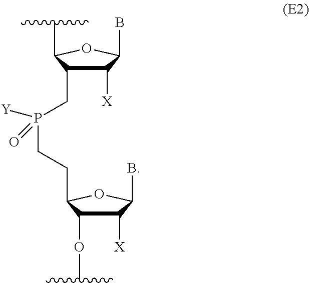

In some embodiments of Formula E1, Z is CH₂ and W is O. In some embodiments, the modified intersubunit linkage of Formula E1 is a modified intersubunit linkage of Formula E3:

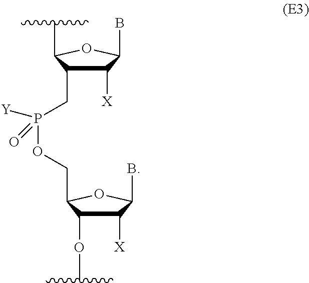

In some embodiments of Formula E1, Z is O and W is CH₂. In some embodiments, the modified intersubunit linkage of Formula E1 is a modified intersubunit linkage of Formula E4:

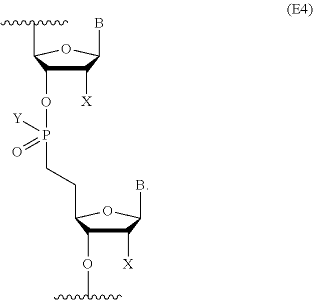

In some embodiments of Formula E1, Z is O and W is CH. In some embodiments, the modified intersubunit linkage of Formula E1 is a modified intersubunit linkage of Formula E5:

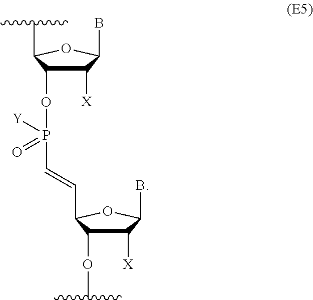

In some embodiments, the modified intersubunit linkage of Formula E1 is a modified intersubunit linkage of Formula E6:

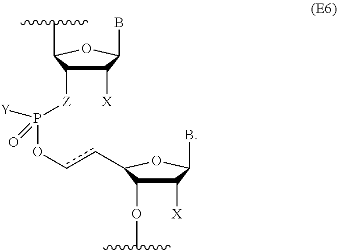

In some embodiments of Formula E6:
each B is, independently, a base pairing moiety;
each X is, independently, selected from the group consisting of halo, hydroxy, and C₁₋₆ alkoxy; optionally wherein each X is, independently, selected from the group consisting of halo (e.g., fluoro) and C₁₋₆ alkoxy (e.g., methoxy, ethoxy, n-propoxy, sec-propoxy, n-butoxy, sec-butoxy, tert-butoxy, n-pentoxy, or n-heptoxy);
Y is selected from the group consisting of O⁻, OH, OR, NH⁻, NH₂, S⁻, and SH, optionally wherein Y is selected from the group consisting of O⁻, OH, and OR;
Z is selected from the group consisting of O and CH₂; and
[symbol] is an optional double bond.

In some embodiments of Formula E6:
each X is, independently, selected from the group consisting of fluoro, hydroxy, and C₁₋₆ alkoxy; optionally wherein each X is, independently, selected from the group consisting of fluoro and C₁₋₆ alkoxy (e.g., methoxy, ethoxy, n-propoxy, sec-propoxy, n-butoxy, sec-butoxy, tert-butoxy, n-pentoxy, or n-heptoxy);
Y is selected from the group consisting of O⁻, OH, and OR;
Z is selected from the group consisting of O and CH₂; and
[symbol] is an optional double bond.

In some embodiments of Formula E6:
each X is, independently, selected from the group consisting of fluoro, hydroxy, methoxy, ethoxy, n-propoxy, sec-propoxy, n-butoxy, sec-butoxy, tert-butoxy, n-pentoxy, and n-heptoxy;
Y is selected from the group consisting of O⁻, OH, and OR;
Z is selected from the group consisting of O and CH₂; and
[symbol] is an optional double bond.

In some embodiments of Formula E6:
each X is, independently, selected from the group consisting of fluoro, hydroxy, methoxy, ethoxy, n-propoxy, sec-propoxy, n-butoxy, sec-butoxy, tert-butoxy, n-pentoxy, and n-heptoxy;
Y is selected from the group consisting of O⁻, OH, and OR;
Z is O; and
[symbol] is an optional double bond.

In some embodiments of Formula E6:
each X is, independently, selected from the group consisting of fluoro, hydroxy, methoxy, ethoxy, n-propoxy, sec-propoxy, n-butoxy, sec-butoxy, tert-butoxy, n-pentoxy, and n-heptoxy;
Y is selected from the group consisting of O⁻, OH, and OR;
Z is CH₂; and
[symbol] is an optional double bond.

In some embodiments of Formula E6:
each X is, independently, selected from the group consisting of fluoro, hydroxy, and methoxy;
Y is selected from the group consisting of O⁻, OH, and OR;
Z is O; and
[symbol] is an optional double bond.

In some embodiments of Formula E6:
each X is, independently, selected from the group consisting of fluoro, hydroxy, and methoxy;
Y is selected from the group consisting of O⁻, OH, and OR;
Z is CH₂; and
[symbol] is an optional double bond.

In some embodiments of Formula E1, Z is O and W is OCH₂. In some embodiments, the modified intersubunit linkage of Formula E1 is a modified intersubunit linkage of Formula E6a:

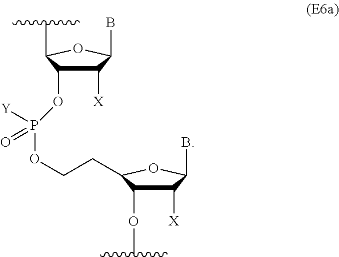

In some embodiments of Formula E1, Z is CH₂ and W is CH. In some embodiments, the modified intersubunit linkage of Formula E1 is a modified intersubunit linkage of Formula E7:

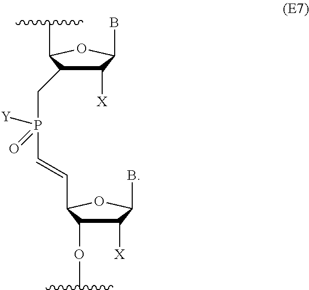

In some embodiments of Formula E1, the base pairing moiety B is selected from the group consisting of adenine, guanine, cytosine, and uracil.

In some embodiments, at least one modified intersubunit linkage is of Formula E8:

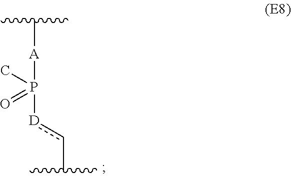

wherein:
D is selected from the group consisting of O, S, B, BR², N, NR², OCH₂, OCH, CH₂, and CH, optionally wherein D is selected from the group consisting of OCH₂ and OCH;
C is selected from the group consisting of O⁻, OH, OR¹, NH⁻, NH₂, S⁻, and SH, optionally wherein C is selected from the group consisting of O⁻, OH, and OR¹;
A is selected from the group consisting of O, S, BR², NR², and CH₂;
R¹ is a protecting group;
each R² is, independently, H or optionally substituted C₁-C₆ alkyl;
[symbol] is an optional double bond; and
the intersubunit is bridging two optionally modified nucleosides.

In some embodiments, D is OCH₂.

In some embodiments, D is OCH and [symbol] is a double bond.

In some embodiments, A is O.

In some embodiments, A is CH₂.

In some embodiments, when C is O⁻, either A or D is not O.

In some embodiments, D is CH₂. In another embodiment, the modified intersubunit linkage of Formula E8 is a modified intersubunit linkage of Formula E9:

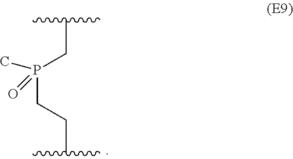 [SMILES: *CCP(C)(=O)C*]

In some embodiments, D is O. In another embodiment, the modified intersubunit linkage of Formula E8 is a modified intersubunit linkage of Formula E10:

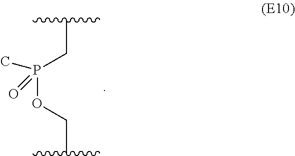 [SMILES: *COP(C)(=O)C*]

In some embodiments, D is CH₂. In another embodiment, the modified intersubunit linkage of Formula E8 is a modified intersubunit linkage of Formula E11:

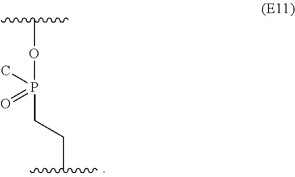 [SMILES: *CCP(C)(=O)O*]

In some embodiments, D is CH. In another embodiment, the modified intersubunit linkage of Formula E8 is a modified intersubunit linkage of Formula E12:

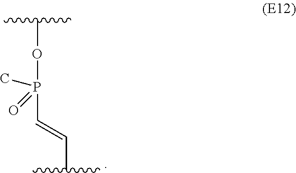 [SMILES: */C=C/P(C)(=O)O*]

In some embodiments, D is OCH₂. In another embodiment, the modified intersubunit linkage of Formula E8 is a modified intersubunit linkage of Formula E13:

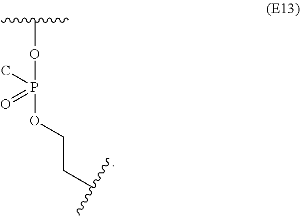 [SMILES: *CCOP(C)(=O)O*]

In another embodiment, the modified intersubunit linkage of Formula E8 is a modified intersubunit linkage of Formula E14:

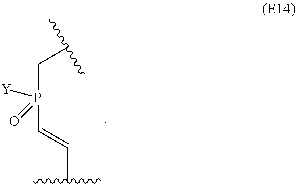

In another embodiment, the modified intersubunit linkage of Formula E8 is a modified intersubunit linkage of Formula E15:

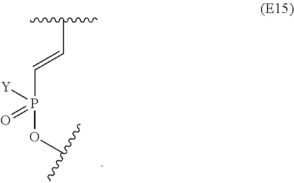

In some embodiments of the modified siRNA linkage, each optionally modified nucleoside is independently, at each occurrence, selected from the group consisting of adenosine, guanosine, cytidine, and uridine.

In some embodiments, at least one modified intersubunit linkage is of Formula E8:

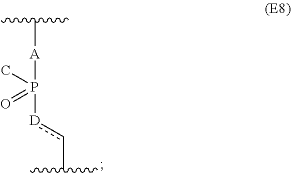

wherein:
D is selected from the group consisting of O, OCH₂, OCH, CH₂, and CH, optionally wherein D is selected from the group consisting of OCH₂ and OCH;
C is selected from the group consisting of O⁻, OH, OR¹, NH⁻, NH₂, S⁻, and SH, optionally wherein C is selected from the group consisting of O⁻, OH, and OR¹;
A is selected from the group consisting of O and CH₂;
R¹ is a protecting group selected from the group consisting of dimethoxytrityl (DMTr), succinate, tert-butyl dimethylsilyl (TBDMS), benzoyl (Bz), benzyl (Bn), methoxyethoxymethyl ether (MOM), methoxybenzyl ether (PMB), methylthiomethyl ether, pivaloyl (Piv), tetrahydropyranyl (THP), tetrahydrofuranyl (THF), trityl (Trt), triisopropylsilyl (TIPS), tert-butyldiphenylsilyl (TBDPS), and acetate;
[symbol] is an optional double bond;
and the intersubunit is bridging two optionally modified nucleosides.

In some embodiments, D is OCH₂.

In some embodiments, D is OCH and [symbol] is a double bond.

In some embodiments, A is O.

In some embodiments, A is CH₂.

In some embodiments of any of the modified intersubunit linkages described above, the base pairing moiety B is selected from the group consisting of adenine, guanine, cytosine, and uracil.

In some embodiments, R is a protecting group selected from the group consisting of dimethoxytrityl (DMTr), succinate, tert-butyl dimethylsilyl (TBDMS), benzoyl (Bz), benzyl (Bn), methoxyethoxymethyl ether (MOM), methoxybenzyl ether (PMB), methylthiomethyl ether, pivaloyl (Piv), tetrahydropyranyl (THP), tetrahydrofuranyl (THF), trityl (Trt), triisopropylsilyl (TIPS), tert-butyldiphenylsilyl (TBDPS), and acetate; and
[symbol] is an optional double bond.

In some embodiments, the second region includes at least one phosphorothioate internucleoside linkage.

In some embodiments, the second region contains at least one nucleotide including a modified ribose. In some embodiments, the second region contains at least one 2′-methoxy nucleotide. In some embodiments, the second region contains at least one 2′-fluoro nucleotide.

In some embodiments, the second region has any one of the following sequences, in the 5′ to 3′ direction:

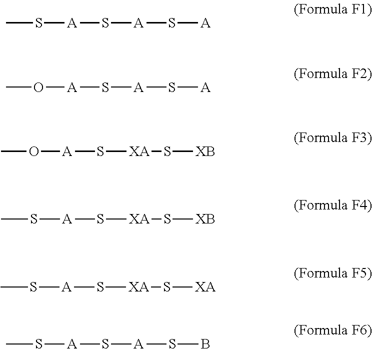

wherein
each S is a phosphorothioate internucleoside linkage;
each O is a phosphodiester internucleoside linkage;
each A is a 2′-methoxy ribonucleoside;
each B is a 2′-fluoro ribonucleoside;
each XA is a 2′-methoxy nucleotide of Formula E6a; and
each XB is a 2′-fluoro nucleotide of Formula E6a. In some embodiments, the second region has any one of the following sequences, in the 5′ to 3′ direction:

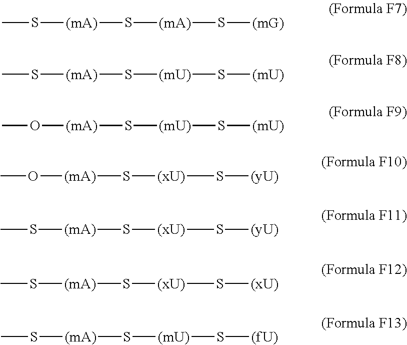

wherein
each S is a phosphorothioate internucleoside linkage,
each O is a phosphodiester internucleoside linkage,
each mA is a 2′-methoxyadenosine,
each mG is a 2′-methoxy guanidine,
each mU is a 2′-methoxyuridine,
each xU is a 2′methoxyuridine of Formula E6a, and
each yU is a 2′-fluorouridine of Formula E6a.

In some embodiments, the antisense strand has a structure represented by Formula I, wherein Formula I is, in the 5′-to-3′ direction:

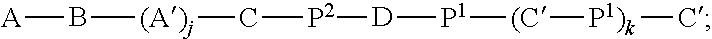

wherein A is represented by the formula C-P¹-D-P¹;
each A′ is represented by the formula C-P²-D-P²;
B is represented by the formula C-P²-D-P²-D-P²-D-P²;
each C is a 2′-O-methyl(2′-O-Me) ribonucleoside;
each C′, independently, is a 2′-O-Me ribonucleoside or a 2′-fluoro (2′-F) ribonucleoside;
each D is a 2′-F ribonucleoside;
each P¹ is a phosphorothioate internucleoside linkage;
each P² is a phosphodiester internucleoside linkage;
j is an integer from 1 to 7 (e.g., 1, 2, 3, 4, 5, 6, or 7); and
k is an integer from 1 to 7 (e.g., 1, 2, 3, 4, 5, 6, or 7).

In some embodiments, the antisense strand has a structure represented by Formula A1, wherein Formula A1 is, in the 5′-to-3′ direction:

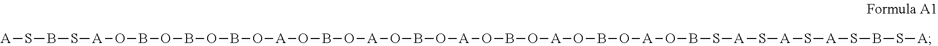

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the antisense strand has a structure represented by Formula II, wherein Formula II is, in the 5′-to-3′ direction:

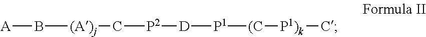

wherein A is represented by the formula C-P¹-D-P¹;
each A′ is represented by the formula C-P²-D-P²;
B is represented by the formula C-P²-D-P²-D-P²-D-P²;
each C is a 2′-O-methyl(2′-O-Me) ribonucleoside;
each C′, independently, is a 2′-O-Me ribonucleoside or a 2′-fluoro (2′-F) ribonucleoside;
each D is a 2′-F ribonucleoside;
each P¹ is a phosphorothioate internucleoside linkage;
each P² is a phosphodiester internucleoside linkage;
j is an integer from 1 to 7 (e.g., 1, 2, 3, 4, 5, 6, or 7); and
k is an integer from 1 to 7 (e.g., 1, 2, 3, 4, 5, 6, or 7).

In some embodiments, antisense strand has a structure represented by Formula A2, wherein Formula A2 is, in the 5′-to-3′ direction:

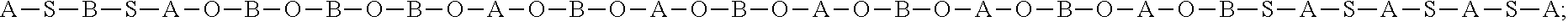

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula III, wherein Formula III is, in the 5′-to-3′ direction:

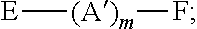

wherein E is represented by the formula (C-P¹)₂;
F is represented by the formula (C-P²)₃-D-P¹-C-P¹-C, (C-P²)₃-D-P²-C-P²-C, (C-P²)₃-D-P¹-C-P¹-D, or (C-P²)₃-D-P²-C-P²-D;
A′, C, D, P¹, and P² are as defined in Formula II; and
m is an integer from 1 to 7 (e.g., 1, 2, 3, 4, 5, 6, or 7).

In some embodiments, the sense strand has a structure represented by Formula S1, wherein Formula S1 is, in the 5′-to-3′ direction:

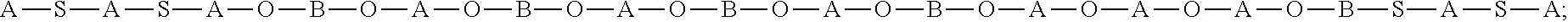

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula S2, wherein Formula S2 is, in the 5′-to-3′ direction:

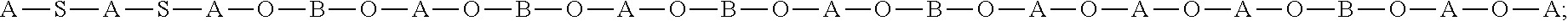

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula S3, wherein Formula S3 is, in the 5′-to-3′ direction:

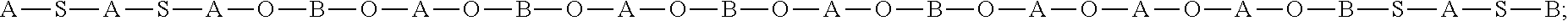

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula S4, wherein Formula S4 is, in the 5′-to-3′ direction:

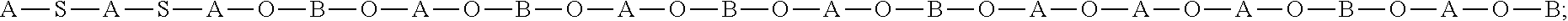

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the antisense strand has a structure represented by Formula IV, wherein Formula IV is, in the 5′-to-3′ direction:

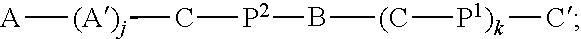

wherein A is represented by the formula C-P¹-D-P¹;
each A′ is represented by the formula C-P²-D-P²;
B is represented by the formula D-P¹-C-P¹-D-P¹;
each C is a 2′-O-Me ribonucleoside;
each C′, independently, is a 2′-O-Me ribonucleoside or a 2′-F ribonucleoside;
each D is a 2′-F ribonucleoside;
each P¹ is a phosphorothioate internucleoside linkage;
each P² is a phosphodiester internucleoside linkage;
j is an integer from 1 to 7 (e.g., 1, 2, 3, 4, 5, 6, or 7); and
k is an integer from 1 to 7 (e.g., 1, 2, 3, 4, 5, 6, or 7).

In some embodiments, the antisense strand has a structure represented by Formula A3, wherein Formula A3 is, in the 5′-to-3′ direction:

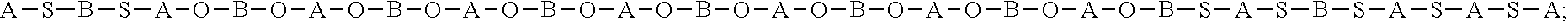

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula V, wherein Formula V is, in the 5′-to-3′ direction:

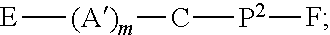

wherein E is represented by the formula (C-P¹)₂;
F is represented by the formula D-P¹-C-P¹-C, D-P²-C-P²-C, D-P¹-C-P¹-D, or D-P²-C-P²-D;
A′, C, D, P¹ and P² are as defined in Formula IV; and
m is an integer from 1 to 7 (e.g., 1, 2, 3, 4, 5, 6, or 7).

In some embodiments, the sense strand has a structure represented by Formula S5, wherein Formula S5 is, in the 5′-to-3′ direction:

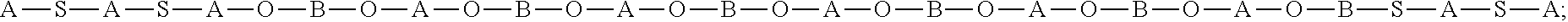

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula S6, wherein Formula S6 is, in the 5′-to-3′ direction:

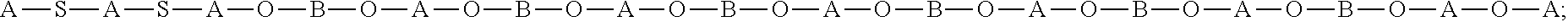

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula S7, wherein Formula S7 is, in the 5′-to-3′ direction:

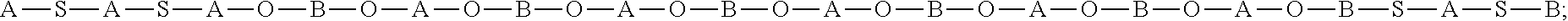

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula S8, wherein Formula S8 is, in the 5′-to-3′ direction:

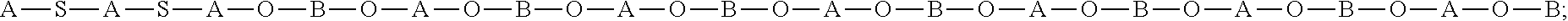

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the antisense strand has a structure represented by Formula VI, wherein Formula VI is, in the 5′-to-3′ direction:

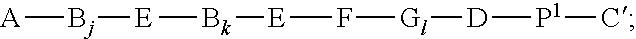

wherein A is represented by the formula C-P¹-D-P¹;
each B is represented by the formula C-P²;
each C is a 2′-O-Me ribonucleoside;
each C′, independently, is a 2′-O-Me ribonucleoside or a 2′-F ribonucleoside;
each D is a 2′-F ribonucleoside;
each E is represented by the formula D-P²-C-P²;
F is represented by the formula D-P¹-C-P¹;
each G is represented by the formula C-P¹;
each P¹ is a phosphorothioate internucleoside linkage;
each P² is a phosphodiester internucleoside linkage;
j is an integer from 1 to 7 (e.g., 1, 2, 3, 4, 5, 6, or 7);
k is an integer from 1 to 7 (e.g., 1, 2, 3, 4, 5, 6, or 7); and
l is an integer from 1 to 7 (e.g., 1, 2, 3, 4, 5, 6, or 7).

In some embodiments, the antisense strand has a structure represented by Formula A4, wherein Formula A4 is, in the 5′-to-3′ direction:

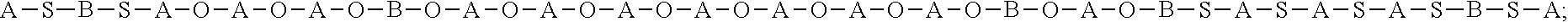

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula VII, wherein Formula VII is, in the 5′-to-3′ direction:

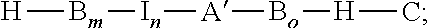

wherein A′ is represented by the formula C-P²-D-P²;
each H is represented by the formula (C-P¹)₂;
each I is represented by the formula (D-P²);
B, C, D, P¹ and P² are as defined in Formula VI;
m is an integer from 1 to 7 (e.g., 1, 2, 3, 4, 5, 6, or 7);
n is an integer from 1 to 7 (e.g., 1, 2, 3, 4, 5, 6, or 7); and
is an integer from 1 to 7 (e.g., 1, 2, 3, 4, 5, 6, or 7).

In some embodiments, the sense strand has a structure represented by Formula S9, wherein Formula S9 is, in the 5′-to-3′ direction:

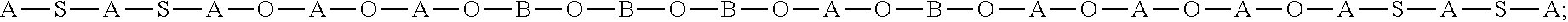

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the antisense strand also has a 5′ phosphorus stabilizing moiety at the 5′ end of the antisense strand.

In some embodiments, the sense strand also has a 5′ phosphorus stabilizing moiety at the 5′ end of the sense strand.

In some embodiments, each 5′ phosphorus stabilizing moiety is, independently, represented by any one of Formulas IX, XX, XI, XII, XIII, XIV, XV, or XVI:

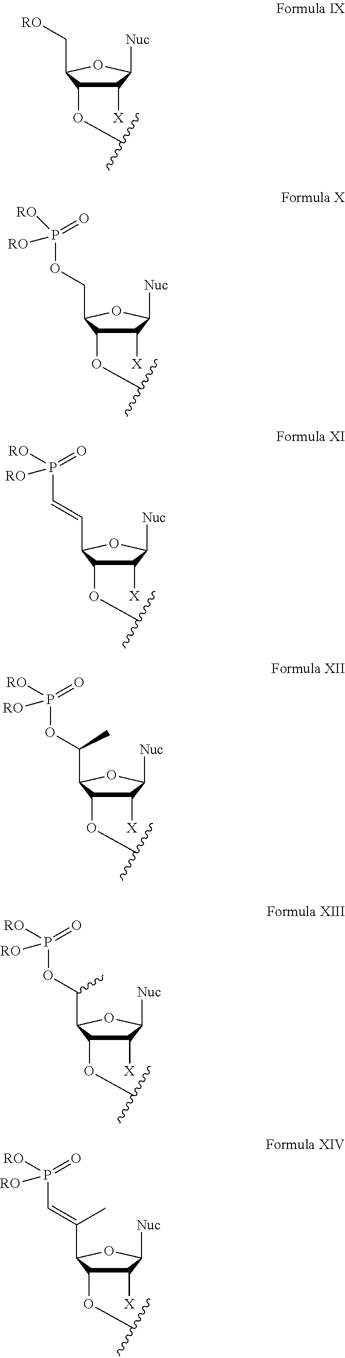

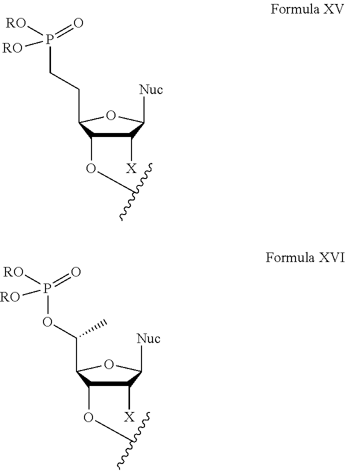

wherein Nuc represents a nucleobase, optionally wherein the nucleobase is selected from the group consisting of adenine, uracil, guanine, thymine, and cytosine, and R represents an optionally substituted alkyl, optionally substituted alkenyl, optionally substituted alkynyl, phenyl, benzyl, a cation (e.g., a monovalent cation), or hydrogen.

In some embodiments, the nucleobase is an adenine, uracil, guanine, thymine, or cytosine.

In some embodiments, the 5′ phosphorus stabilizing moiety is (E)-vinylphosphonate represented by Formula XI.

In some embodiments, the siRNA molecule also has a hydrophobic moiety at the 5′ or the 3′ end of the siRNA molecule.

In some embodiments, the hydrophobic moiety is selected from a group consisting of cholesterol, vitamin D, and tocopherol.

In some embodiments, the siRNA molecule is a branched siRNA molecule.

In some embodiments, the branched siRNA molecule is di-branched, tri-branched, or tetra-branched.

In some embodiments, the siRNA molecule is di-branched, optionally wherein the di-branched siRNA molecule is represented by any one of Formulas XVII, XVIII, or XIX:

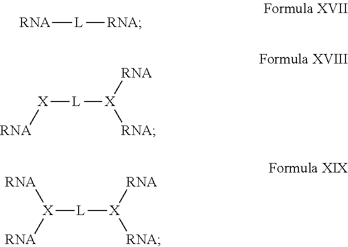

wherein each RNA is, independently, an siRNA molecule, L is a linker, and each X, independently, represents a branch point moiety.

In some embodiments, the di-branched siRNA molecule is represented by Formula XVII. In some embodiments, the di-branched siRNA molecule is represented by Formula XVIII. In some embodiments, the di-branched siRNA molecule is represented by Formula XIX.

In some embodiments, the siRNA molecule is tri-branched, optionally wherein the tri-branched siRNA molecule is represented by any one of Formulas XX, XXI, XXII, or XXIII:

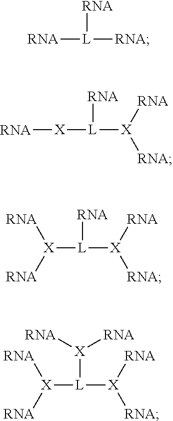

wherein each RNA is, independently, an siRNA molecule, L is a linker, and each X, independently, represents a branch point moiety.

In some embodiments, the tri-branched siRNA molecule is represented by Formula XX. In some embodiments, the tri-branched siRNA molecule is represented by Formula XXI. In some embodiments, the tri-branched siRNA molecule is represented by Formula XXII. In some embodiments, the tri-branched siRNA molecule is represented by Formula XXIII.

In some embodiments, the siRNA molecule is tetra-branched, optionally wherein the tetra-branched siRNA molecule is represented by any one of Formulas XXIV, XXV, XXVI, XXVII, or XXVIII:

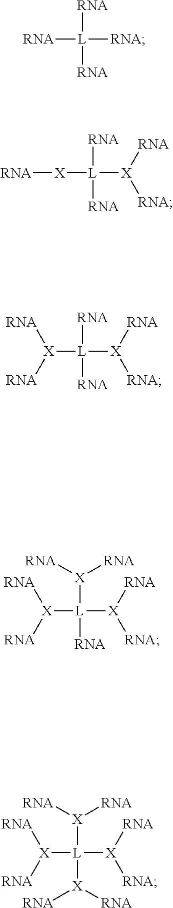

wherein each RNA is, independently, an siRNA molecule, L is a linker, and each X, independently, represents a branch point moiety.

In some embodiments, the tetra-branched siRNA molecule is represented by Formula XXIV. In some embodiments, the tetra-branched siRNA molecule is represented by Formula XXV. In some embodiments, the tetra-branched siRNA molecule is represented by Formula XXVI. In some embodiments, the tetra-branched siRNA molecule is represented by Formula XXVII. In some embodiments, the tetra-branched siRNA molecule is represented by Formula XXVIII.

In some embodiments of the branched siRNA, the linker is selected from a group consisting of one or more contiguous subunits of an ethylene glycol (e.g., polyethylene glycol (PEG), such as, e.g., triethylene glycol (TrEG) or tetraethylene glycol (TEG)), alkyl, carbohydrate, block copolymer, peptide, RNA, and DNA.

In some embodiments, the linker is an ethylene glycol oligomer. In some embodiments, the linker is an alkyl oligomer. In some embodiments, the linker is a carbohydrate oligomer. In some embodiments, the linker is a block copolymer. In some embodiments, the linker is a peptide oligomer. In some embodiments, the linker is an RNA oligomer. In some embodiments, the linker is a DNA oligomer.

In some embodiments, the ethylene glycol oligomer is a PEG. In some embodiments, the PEG is a TrEG. In some embodiments, the PEG is a TEG.

In some embodiments, the oligomer or copolymer contains 2 to 20 contiguous subunits (e.g., 2, 3, 4, 5, 6, 7, 8, 9, 10, 11, 12, 13, 14, 15, 16, 17, 18, 19, or 20 contiguous subunits).

In some embodiments, the linker attaches one or more (e.g., 1, 2, 3, 4, or more) siRNA molecules by way of a covalent bond-forming moiety.

In some embodiments, the covalent bond-forming moiety is selected from the group consisting of an alkyl, ester, amide, carbamate, phosphonate, phosphate, phosphorothioate, phosphoroamidate, triazole, urea, and formacetal.

In some embodiments, the linker includes a structure of Formula L1:

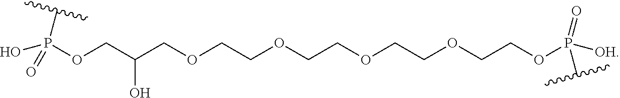 [SMILES: *P(=O)(O)OCCOCCOCCOCCOCC(O)COP(*)(=O)O]

In some embodiments, the linker includes a structure of Formula L2:

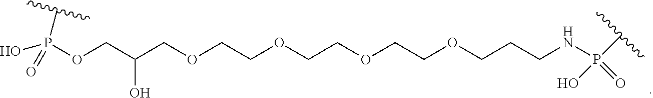 [SMILES: *P(=O)(O)NCCCOCCOCCOCCOCC(O)COP(*)(=O)O]

In some embodiments, the linker includes a structure of Formula L3:

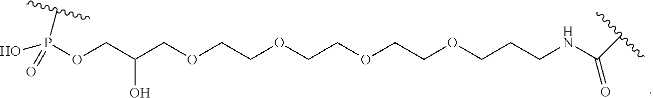 [SMILES: *C(=O)NCCCOCCOCCOCCOCC(O)COP(*)(=O)O]

In some embodiments, the linker includes a structure of Formula L4:

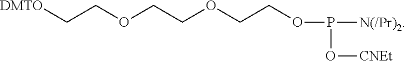

In some embodiments, the linker includes a structure of Formula L5:

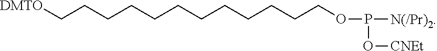

In some embodiments, the linker includes a structure of Formula L6:

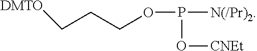

In some embodiments, the linker includes a structure of Formula L7:

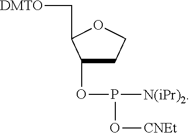

In some embodiments, the linker includes a structure of Formula L8:

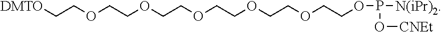

In some embodiments, the linker includes a structure of Formula L9:

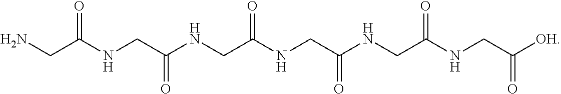 [SMILES: NCC(=O)NCC(=O)NCC(=O)NCC(=O)NCC(=O)NCC(=O)O]

In some embodiments of any of the siRNA molecules described herein, 50% or more of the ribonucleotides in the antisense strand are 2′-O-Me ribonucleotides (e.g., 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the ribonucleotides in the antisense strand may be 2′-O-Me ribonucleotides).

In some embodiments, 60% or more of the ribonucleotides in the antisense strand are 2′-O-Me ribonucleotides (e.g., 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the ribonucleotides in the antisense strand may be 2′-O-Me ribonucleotides).

In some embodiments, 70% or more of the ribonucleotides in the antisense strand are 2′-O-Me ribonucleotides (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the ribonucleotides in the antisense strand may be 2′-O-Me ribonucleotides).

In some embodiments, 80% or more of the ribonucleotides in the antisense strand are 2′-O-Me ribonucleotides (e.g., 80%, 81%, 82%, 83%, 84%, 85%, 86%, 87%, 88%, 89%, 90%, 91%, 92%, 93%, 94%, 95%, 96%, 97%, 98%, 99%, or 100% of the ribonucleotides in the antisense strand may be 2′-O-Me ribonucleotides).

In some embodiments, 90% or more of the ribonucleotides in the antisense strand are 2′-O-Me ribonucleotides (e.g., 90%, 91%, 92%, 93%, 94%, 95%, 96%, 97%, 98%, 99%, or 100% of the ribonucleotides in the antisense strand may be 2′-O-Me ribonucleotides).

In some embodiments, 10% or less of the internucleoside linkages are phosphodiester linkages or phosphorothioate linkages. In some embodiments, at least 10%, 20%, 30%, 40%, 50%, 60%, 70%, 80%, or 90% of the internucleoside linkages are phosphodiester linkages or phosphorothioate linkages. In some embodiments, 100% of the internucleoside linkages are phosphodiester linkages or phosphorothioate linkages.

In some embodiments, 9 internucleoside linkages are phosphodiester linkages or phosphorothioate linkages.

In some embodiments, the length of the antisense strand is between 10 and 30 nucleotides (e.g., 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides), 15 and 25 nucleotides (e.g., 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, or 25 nucleotides), or 18 and 23 nucleotides (e.g., 18 nucleotides, 19 nucleotides, 20 nucleotides, 21 nucleotides, 22 nucleotides, or 23 nucleotides). In some embodiments, the length of the antisense strand is 20 nucleotides. In some embodiments, the length of the antisense strand is 21 nucleotides. In some embodiments, the length of the antisense strand is 22 nucleotides. In some embodiments, the length of the antisense strand is 23 nucleotides. In some embodiments, the length of the antisense strand is 24 nucleotides. In some embodiments, the length of the antisense strand is 25 nucleotides. In some embodiments, the length of the antisense strand is 26 nucleotides. In some embodiments, the length of the antisense strand is 27 nucleotides. In some embodiments, the length of the antisense strand is 28 nucleotides. In some embodiments, the length of the antisense strand is 29 nucleotides. In some embodiments, the length of the antisense strand is 30 nucleotides.

In some embodiments, the siRNA molecules of the branched compound are joined to one another by way of a linker (e.g., an ethylene glycol oligomer, such as tetraethylene glycol). In some embodiments, the siRNA molecules of the branched compound are joined to one another by way of a linker between the sense strand of one siRNA molecule and the sense strand of the other siRNA molecule. In some embodiments, the siRNA molecules are joined by way of linkers between the antisense strand of one siRNA molecule and the antisense strand of the other siRNA molecule. In some embodiments, the siRNA molecules of the branched compound are joined to one another by way of a linker between the sense strand of one siRNA molecule and the antisense strand of the other siRNA molecule.

In some embodiments, the length of the sense strand is between 12 and 30 nucleotides (e.g., 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides), or 14 and 18 nucleotides (e.g., 14 nucleotides, 15 nucleotides, 16 nucleotides, 17 nucleotides, or 18 nucleotides). In some embodiments, the length of the sense strand is 15 nucleotides. In some embodiments, the length of the sense strand is 16 nucleotides. In some embodiments, the length of the sense strand is 17 nucleotides. In some embodiments, the length of the sense strand is 18 nucleotides. In some embodiments, the length of the sense strand is 19 nucleotides. In some embodiments, the length of the sense strand is 20 nucleotides. In some embodiments, the length of the sense strand is 21 nucleotides. In some embodiments, the length of the sense strand is 22 nucleotides. In some embodiments, the length of the sense strand is 23 nucleotides. In some embodiments, the length of the sense strand is 24 nucleotides. In some embodiments, the length of the sense strand is 25 nucleotides. In some embodiments, the length of the sense strand is 26 nucleotides. In some embodiments, the length of the sense strand is 27 nucleotides. In some embodiments, the length of the sense strand is 28 nucleotides. In some embodiments, the length of the sense strand is 29 nucleotides. In some embodiments, the length of the sense strand is 30 nucleotides.

In some embodiments, four internucleoside linkages are phosphorothioate linkages.

In some embodiments of the siRNA molecules described herein, the antisense strand is 18 nucleotides in length and the sense strand is 14 nucleotides in length. In some embodiments, the antisense strand is 18 nucleotides in length and the sense strand is 15 nucleotides in length. In some embodiments, the antisense strand is 18 nucleotides in length and the sense strand is 16 nucleotides in length. In some embodiments, the antisense strand is 18 nucleotides in length and the sense strand is 17 nucleotides in length. In some embodiments, the antisense strand is 18 nucleotides in length and the sense strand is 18 nucleotides in length. In some embodiments, the antisense strand is 19 nucleotides in length and the sense strand is 14 nucleotides in length. In some embodiments, the antisense strand is 19 nucleotides in length and the sense strand is 15 nucleotides in length. In some embodiments, the antisense strand is 19 nucleotides in length and the sense strand is 16 nucleotides in length. In some embodiments, the antisense strand is 19 nucleotides in length and the sense strand is 17 nucleotides in length. In some embodiments, the antisense strand is 19 nucleotides in length and the sense strand is 18 nucleotides in length. In some embodiments, the antisense strand is 19 nucleotides in length and the sense strand is 19 nucleotides in length. In some embodiments, the antisense strand is 20 nucleotides in length and the sense strand is 14 nucleotides in length. In some embodiments, the antisense strand is 20 nucleotides in length and the sense strand is 15 nucleotides in length. In some embodiments, the antisense strand is 20 nucleotides in length and the sense strand is 16 nucleotides in length. In some embodiments, the antisense strand is 20 nucleotides in length and the sense strand is 17 nucleotides in length. In some embodiments, the antisense strand is 20 nucleotides in length and the sense strand is 18 nucleotides in length. In some embodiments, the antisense strand is 20 nucleotides in length and the sense strand is 19 nucleotides in length. In some embodiments, the antisense strand is 20 nucleotides in length and the sense strand is 20 nucleotides in length. In some embodiments, the antisense strand is 21 nucleotides in length and the sense strand is 14 nucleotides in length. In some embodiments, the antisense strand is 21 nucleotides in length and the sense strand is 15 nucleotides in length. In some embodiments, the antisense strand is 21 nucleotides in length and the sense strand is 16 nucleotides in length. In some embodiments, the antisense strand is 21 nucleotides in length and the sense strand is 17 nucleotides in length. In some embodiments, the antisense strand is 21 nucleotides in length and the sense strand is 18 nucleotides in length. In some embodiments, the antisense strand is 21 nucleotides in length and the sense strand is 19 nucleotides in length. In some embodiments, the antisense strand is 21 nucleotides in length and the sense strand is 20 nucleotides in length. In some embodiments, the antisense strand is 21 nucleotides in length and the sense strand is 21 nucleotides in length. In some embodiments, the antisense strand is 22 nucleotides in length and the sense strand is 14 nucleotides in length. In some embodiments, the antisense strand is 22 nucleotides in length and the sense strand is 15 nucleotides in length. In some embodiments, the antisense strand is 22 nucleotides in length and the sense strand is 16 nucleotides in length. In some embodiments, the antisense strand is 22 nucleotides in length and the sense strand is 17 nucleotides in length. In some embodiments, the antisense strand is 22 nucleotides in length and the sense strand is 18 nucleotides in length. In some embodiments, the antisense strand is 22 nucleotides in length and the sense strand is 19 nucleotides in length. In some embodiments, the antisense strand is 22 nucleotides in length and the sense strand is 20 nucleotides in length. In some embodiments, the antisense strand is 22 nucleotides in length and the sense strand is 21 nucleotides in length. In some embodiments, the antisense strand is 22 nucleotides in length and the sense strand is 22 nucleotides in length. In some embodiments, the antisense strand is 23 nucleotides in length and the sense strand is 14 nucleotides in length. In some embodiments, the antisense strand is 23 nucleotides in length and the sense strand is 15 nucleotides in length. In some embodiments, the antisense strand is 23 nucleotides in length and the sense strand is 16 nucleotides in length. In some embodiments, the antisense strand is 23 nucleotides in length and the sense strand is 17 nucleotides in length. In some embodiments, the antisense strand is 23 nucleotides in length and the sense strand is 18 nucleotides in length. In some embodiments, the antisense strand is 23 nucleotides in length and the sense strand is 19 nucleotides in length. In some embodiments, the antisense strand is 23 nucleotides in length and the sense strand is 20 nucleotides in length. In some embodiments, the antisense strand is 23 nucleotides in length and the sense strand is 21 nucleotides in length. In some embodiments, the antisense strand is 23 nucleotides in length and the sense strand is 22 nucleotides in length. In some embodiments, the antisense strand is 23 nucleotides in length and the sense strand is 23 nucleotides in length. In some embodiments, the antisense strand is 24 nucleotides in length and the sense strand is 14 nucleotides in length. In some embodiments, the antisense strand is 24 nucleotides in length and the sense strand is 15 nucleotides in length. In some embodiments, the antisense strand is 24 nucleotides in length and the sense strand is 16 nucleotides in length. In some embodiments, the antisense strand is 24 nucleotides in length and the sense strand is 17 nucleotides in length. In some embodiments, the antisense strand is 24 nucleotides in length and the sense strand is 18 nucleotides in length. In some embodiments, the antisense strand is 24 nucleotides in length and the sense strand is 19 nucleotides in length. In some embodiments, the antisense strand is 24 nucleotides in length and the sense strand is 20 nucleotides in length. In some embodiments, the antisense strand is 24 nucleotides in length and the sense strand is 21 nucleotides in length. In some embodiments, the antisense strand is 24 nucleotides in length and the sense strand is 22 nucleotides in length. In some embodiments, the antisense strand is 24 nucleotides in length and the sense strand is 23 nucleotides in length. In some embodiments, the antisense strand is 24 nucleotides in length and the sense strand is 24 nucleotides in length. In some embodiments, the antisense strand is 25 nucleotides in length and the sense strand is 14 nucleotides in length. In some embodiments, the antisense strand is 25 nucleotides in length and the sense strand is 15 nucleotides in length. In some embodiments, the antisense strand is 25 nucleotides in length and the sense strand is 16 nucleotides in length. In some embodiments, the antisense strand is 25 nucleotides in length and the sense strand is 17 nucleotides in length. In some embodiments, the antisense strand is 25 nucleotides in length and the sense strand is 18 nucleotides in length. In some embodiments, the antisense strand is 25 nucleotides in length and the sense strand is 19 nucleotides in length. In some embodiments, the antisense strand is 25 nucleotides in length and the sense strand is 20 nucleotides in length. In some embodiments, the antisense strand is 25 nucleotides in length and the sense strand is 21 nucleotides in length. In some embodiments, the antisense strand is 25 nucleotides in length and the sense strand is 22 nucleotides in length. In some embodiments, the antisense strand is 25 nucleotides in length and the sense strand is 23 nucleotides in length. In some embodiments, the antisense strand is 25 nucleotides in length and the sense strand is 24 nucleotides in length. In some embodiments, the antisense strand is 25 nucleotides in length and the sense strand is 25 nucleotides in length. In some embodiments, the antisense strand is 26 nucleotides in length and the sense strand is 14 nucleotides in length. In some embodiments, the antisense strand is 26 nucleotides in length and the sense strand is 15 nucleotides in length. In some embodiments, the antisense strand is 26 nucleotides in length and the sense strand is 16 nucleotides in length. In some embodiments, the antisense strand is 26 nucleotides in length and the sense strand is 17 nucleotides in length. In some embodiments, the antisense strand is 26 nucleotides in length and the sense strand is 18 nucleotides in length. In some embodiments, the antisense strand is 26 nucleotides in length and the sense strand is 19 nucleotides in length. In some embodiments, the antisense strand is 26 nucleotides in length and the sense strand is 20 nucleotides in length. In some embodiments, the antisense strand is 26 nucleotides in length and the sense strand is 21 nucleotides in length. In some embodiments, the antisense strand is 26 nucleotides in length and the sense strand is 22 nucleotides in length. In some embodiments, the antisense strand is 26 nucleotides in length and the sense strand is 23 nucleotides in length. In some embodiments, the antisense strand is 26 nucleotides in length and the sense strand is 24 nucleotides in length. In some embodiments, the antisense strand is 26 nucleotides in length and the sense strand is 25 nucleotides in length. In some embodiments, the antisense strand is 26 nucleotides in length and the sense strand is 26 nucleotides in length. In some embodiments, the antisense strand is 27 nucleotides in length and the sense strand is 14 nucleotides in length. In some embodiments, the antisense strand is 27 nucleotides in length and the sense strand is 15 nucleotides in length. In some embodiments, the antisense strand is 27 nucleotides in length and the sense strand is 16 nucleotides in length. In some embodiments, the antisense strand is 27 nucleotides in length and the sense strand is 17 nucleotides in length. In some embodiments, the antisense strand is 27 nucleotides in length and the sense strand is 18 nucleotides in length. In some embodiments, the antisense strand is 27 nucleotides in length and the sense strand is 19 nucleotides in length. In some embodiments, the antisense strand is 27 nucleotides in length and the sense strand is 20 nucleotides in length. In some embodiments, the antisense strand is 27 nucleotides in length and the sense strand is 21 nucleotides in length. In some embodiments, the antisense strand is 27 nucleotides in length and the sense strand is 22 nucleotides in length. In some embodiments, the antisense strand is 27 nucleotides in length and the sense strand is 23 nucleotides in length. In some embodiments, the antisense strand is 27 nucleotides in length and the sense strand is 24 nucleotides in length. In some embodiments, the antisense strand is 27 nucleotides in length and the sense strand is 25 nucleotides in length. In some embodiments, the antisense strand is 27 nucleotides in length and the sense strand is 26 nucleotides in length. In some embodiments, the antisense strand is 27 nucleotides in length and the sense strand is 27 nucleotides in length. In some embodiments, the antisense strand is 28 nucleotides in length and the sense strand is 14 nucleotides in length. In some embodiments, the antisense strand is 28 nucleotides in length and the sense strand is 15 nucleotides in length. In some embodiments, the antisense strand is 28 nucleotides in length and the sense strand is 16 nucleotides in length. In some embodiments, the antisense strand is 28 nucleotides in length and the sense strand is 17 nucleotides in length. In some embodiments, the antisense strand is 28 nucleotides in length and the sense strand is 18 nucleotides in length. In some embodiments, the antisense strand is 28 nucleotides in length and the sense strand is 19 nucleotides in length. In some embodiments, the antisense strand is 28 nucleotides in length and the sense strand is 20 nucleotides in length. In some embodiments, the antisense strand is 28 nucleotides in length and the sense strand is 21 nucleotides in length. In some embodiments, the antisense strand is 28 nucleotides in length and the sense strand is 22 nucleotides in length. In some embodiments, the antisense strand is 28 nucleotides in length and the sense strand is 23 nucleotides in length. In some embodiments, the antisense strand is 28 nucleotides in length and the sense strand is 24 nucleotides in length. In some embodiments, the antisense strand is 28 nucleotides in length and the sense strand is 25 nucleotides in length. In some embodiments, the antisense strand is 28 nucleotides in length and the sense strand is 26 nucleotides in length. In some embodiments, the antisense strand is 28 nucleotides in length and the sense strand is 27 nucleotides in length. In some embodiments, the antisense strand is 28 nucleotides in length and the sense strand is 28 nucleotides in length. In some embodiments, the antisense strand is 29 nucleotides in length and the sense strand is 14 nucleotides in length. In some embodiments, the antisense strand is 29 nucleotides in length and the sense strand is 15 nucleotides in length. In some embodiments, the antisense strand is 29 nucleotides in length and the sense strand is 16 nucleotides in length. In some embodiments, the antisense strand is 29 nucleotides in length and the sense strand is 17 nucleotides in length. In some embodiments, the antisense strand is 29 nucleotides in length and the sense strand is 18 nucleotides in length. In some embodiments, the antisense strand is 29 nucleotides in length and the sense strand is 19 nucleotides in length. In some embodiments, the antisense strand is 29 nucleotides in length and the sense strand is 20 nucleotides in length. In some embodiments, the antisense strand is 29 nucleotides in length and the sense strand is 21 nucleotides in length. In some embodiments, the antisense strand is 29 nucleotides in length and the sense strand is 22 nucleotides in length. In some embodiments, the antisense strand is 29 nucleotides in length and the sense strand is 23 nucleotides in length. In some embodiments, the antisense strand is 29 nucleotides in length and the sense strand is 24 nucleotides in length. In some embodiments, the antisense strand is 29 nucleotides in length and the sense strand is 25 nucleotides in length. In some embodiments, the antisense strand is 29 nucleotides in length and the sense strand is 26 nucleotides in length. In some embodiments, the antisense strand is 29 nucleotides in length and the sense strand is 27 nucleotides in length. In some embodiments, the antisense strand is 29 nucleotides in length and the sense strand is 28 nucleotides in length. In some embodiments, the antisense strand is 29 nucleotides in length and the sense strand is 29 nucleotides in length. In some embodiments, the antisense strand is 30 nucleotides in length and the sense strand is 14 nucleotides in length. In some embodiments, the antisense strand is 30 nucleotides in length and the sense strand is 15 nucleotides in length. In some embodiments, the antisense strand is 30 nucleotides in length and the sense strand is 16 nucleotides in length. In some embodiments, the antisense strand is 30 nucleotides in length and the sense strand is 17 nucleotides in length. In some embodiments, the antisense strand is 30 nucleotides in length and the sense strand is 18 nucleotides in length. In some embodiments, the antisense strand is 30 nucleotides in length and the sense strand is 19 nucleotides in length. In some embodiments, the antisense strand is 30 nucleotides in length and the sense strand is 20 nucleotides in length. In some embodiments, the antisense strand is 30 nucleotides in length and the sense strand is 21 nucleotides in length. In some embodiments, the antisense strand is 30 nucleotides in length and the sense strand is 22 nucleotides in length. In some embodiments, the antisense strand is 30 nucleotides in length and the sense strand is 23 nucleotides in length. In some embodiments, the antisense strand is 30 nucleotides in length and the sense strand is 24 nucleotides in length. In some embodiments, the antisense strand is 30 nucleotides in length and the sense strand is 25 nucleotides in length. In some embodiments, the antisense strand is 30 nucleotides in length and the sense strand is 26 nucleotides in length. In some embodiments, the antisense strand is 30 nucleotides in length and the sense strand is 27 nucleotides in length. In some embodiments, the antisense strand is 30 nucleotides in length and the sense strand is 28 nucleotides in length. In some embodiments, the antisense strand is 30 nucleotides in length and the sense strand is 29 nucleotides in length. In some embodiments, the antisense strand is 30 nucleotides in length and the sense strand is 30 nucleotides in length.

In a further aspect, the disclosure provides a pharmaceutical composition containing an siRNA molecule of any of the preceding aspects or embodiments of the disclosure, and a pharmaceutically acceptable excipient, carrier, or diluent.

In a further aspect, the disclosure provides a method of delivering an siRNA molecule to the central nervous system (CNS) of a subject by administering the multimeric oligonucleotide, composition of siRNA molecules, or pharmaceutical composition of any of the foregoing aspects or embodiments of the disclosure to the CNS of the subject.

In some embodiments, the multimeric oligonucleotide, composition of siRNA molecules, or pharmaceutical composition is administered to the subject by way of intrastriatal, intracerebroventricular, or intrathecal injection.

In some embodiments, the delivering of the siRNA molecule or pharmaceutical composition to the CNS of the subject results in gene silencing of a target gene in the subject.

In some embodiments, the target gene is an overactive disease driver. In some embodiments, the target gene is a negative regulator of a gene with reduced expression that is associated with a disease state in the subject. In some embodiments, the target gene is a positive regulator of a gene with increased expression that is associated with a disease state in the subject. In some embodiments, the target gene is a splice isoform of the target gene, wherein the splice isoform reduces expression of the target gene.

In some embodiments, the gene silencing treats a disease state in the subject,

In some embodiments of the methods described herein, the siRNA molecule or the pharmaceutical composition is administered to the subject by way of intracerebroventricular, intrastriatal, intraparenchymal or intrathecal injection. In some embodiments, the siRNA molecule or the pharmaceutical composition is administered to the subject by way of intravenous, intramuscular, or subcutaneous injection.

In some embodiments of any of the methods described herein, the subject is a human.

In another aspect, the disclosure provides a kit containing an siRNA molecule or pharmaceutical composition of any of the preceding aspects or embodiments of the disclosure, and a package insert that instructs a user of the kit to perform the method of any of the preceding aspects or embodiments of the disclosure.

### Definitions

Unless otherwise defined herein, scientific, and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

As used herein, the term “nucleic acids” refers to RNA or DNA molecules consisting of a chain of ribonucleotides or deoxyribonucleotides, respectively.

As used herein, the term “therapeutic nucleic acid” refers to a nucleic acid molecule (e.g., ribonucleic acid) that has partial or complete complementarity to, and interacts with, a disease-associated target mRNA and mediates silencing of expression of the mRNA.

As used herein, the term “carrier nucleic acid” refers to a nucleic acid molecule (e.g., ribonucleic acid) that has sequence complementarity with, and hybridizes with, a therapeutic nucleic acid. As used herein, the term “3′ end” refers to the end of the nucleic acid that contains an unmodified hydroxyl group at the 3′ carbon of the ribose ring.

As used herein, the term “nucleoside” refers to a molecule made up of a heterocyclic base and its sugar.

As used herein, the term “nucleotide” refers to a nucleoside having a phosphate group, or a variant thereof, on its 3′ or 5′ sugar hydroxyl group. Examples of phosphate group variants include, but are not limited to, saturated alkyl phosphonates, unsaturated alkenyl phosphonates, phosphorothioates, and phosphoramidites.

The term “fixed” when used in the context of a region of an siRNA molecule, a region of nucleobases, a nucleobase sequence, a region of nucleotides, or a nucleotide sequence refers to a sequence of nucleotides that are included in an siRNA molecule (e.g., in the antisense strand of the siRNA molecule) that are independent of the sequence of the target mRNA transcript. The fixed region may be of any suitable length while still maintaining the silencing effect of the siRNA molecule. Such fixed sequences may contain one or more mismatches relative to the target mRNA transcript but may also contain one or more sequence matched nucleotides. That is, the fixed region has a defined sequence of nucleotides that can be inserted into any siRNA molecule regardless of the sequence of the target gene of interest. The fixed region may be a region of contiguous or linked nucleotides, though it need not be. For example, the fixed region may be a region of 1 to 10 (e.g., 1, 2, 3, 4, 5, 6, 7, 8, 9, or 10) contiguous nucleotides with a sequence independent of the target mRNA transcript. Alternatively, there may be sequence matched nucleotides (that is, nucleotides that are complementary to the target mRNA transcript) intervening with nucleotides that are independent of the target mRNA transcript. For example, there may be one or more mismatched nucleotides, followed by one or more sequence matched nucleotides, followed by one or more mismatched nucleotides, or any other permutation of matched and mismatched nucleotides.

As used herein, the term “contiguous” refers to nucleotides that are linked to one another by way of a direct covalent bond.

As used herein, the term “linked” refers to both contiguous nucleotides as defined herein as well as nucleotides joined by way of a linker. The linker may be, for example, an alkyl chain (e.g., methylene, ethylene, propylene, or any larger linear or branched alkylene group), a polyethylene glycol chain (e.g., a TrEG or TEG linker), or any other linker described herein or known in the art.

A “target mRNA transcript” refers to an mRNA transcript that has complementarity sufficient to hybridize to the antisense strand of an siRNA molecule of the disclosure and thereby undergo gene silencing.

“RISC” refers to the RNA-induced silencing complex that mediates RNA interference. The term as used herein may refer to any RISC complex from any organism.

The term “cleavage products” refers to the degraded mRNA after interaction with the RISC complex, after which the RISC complex is free to interact with another mRNA molecule.

In the context of this disclosure, the term “oligonucleotide” refers to an oligomer or polymer of ribonucleic acid (RNA) or deoxyribonucleic acid (DNA) or mimetics thereof. This term includes oligonucleotides composed of naturally-occurring nucleobases, sugars and covalent internucleoside (backbone) linkages as well as oligonucleotides having non-naturally-occurring (e.g., modified) portions that function similarly. Such modified or substituted oligonucleotides are often preferred over native forms because of desirable properties such as, for example, enhanced cellular uptake, enhanced affinity for nucleic acid target and increased stability in the presence of nucleases.

As used herein, the term “siRNA” refers to small interfering RNA duplexes that induce the RNA interference (RNAi) pathway. siRNA molecules may vary in length (generally, between 10 and 30 base pairs) and may contain varying degrees of complementarity to their target mRNA. The term “siRNA” includes duplexes of two separate strands, as well as single strands that optionally form hairpin structures including a duplex region.

As used herein, the term “antisense strand” refers to the strand of the siRNA duplex that contains some degree of complementarity to the target gene.

As used herein, the term “sense strand” refers to the strand of the siRNA duplex that contains complementarity to the antisense strand.

As used herein, the term “overhang” refers to a single-stranded portion of a nucleic acid molecule that is otherwise double-stranded (e.g., a double-stranded siRNA) located at one or both termini (i.e., at the 5′ and/or 3′ terminus).

The term “interfering RNA molecule” refers to an RNA molecule, such as a small interfering RNA (siRNA), microRNA (miRNA), short hairpin RNA (shRNA), or an antisense oligonucleotide (ASO) that suppresses the endogenous function of a target RNA transcript.

As used herein, the terms “express” and “expression” refer to one or more of the following events: (1) production of an RNA template from a DNA sequence (e.g., by transcription); (2) processing of an RNA transcript (e.g., by splicing, editing, 5′ cap formation, and/or 3′ end processing); and (3) translation of an RNA into a polypeptide or protein. In the context of a gene that encodes a protein product, the terms “gene expression” and the like are used interchangeably with the terms “protein expression” and the like. Expression of a gene or protein of interest in a patient can manifest, for example, by detecting: an increase in the quantity or concentration of mRNA encoding corresponding protein (as assessed, e.g., using RNA detection procedures described herein or known in the art, such as quantitative polymerase chain reaction (qPCR) and RNA seq techniques), an increase in the quantity or concentration of the corresponding protein (as assessed, e.g., using protein detection methods described herein or known in the art, such as enzyme-linked immunosorbent assays (ELISA), among others), and/or an increase in the activity of the corresponding protein (e.g., in the case of an enzyme, as assessed using an enzymatic activity assay described herein or known in the art) in a sample obtained from the patient. As used herein, a cell is considered to “express” a gene or protein of interest if one or more, or all, of the above events can be detected in the cell or in a medium in which the cell resides. For example, a gene or protein of interest is considered to be “expressed” by a cell or population of cells if one can detect (i) production of a corresponding RNA transcript, such as an mRNA template, by the cell or population of cells (e.g., using RNA detection procedures described herein); (ii) processing of the RNA transcript (e.g., splicing, editing, 5′ cap formation, and/or 3′ end processing, such as using RNA detection procedures described herein); (iii) translation of the RNA template into a protein product (e.g., using protein detection procedures described herein); and/or (iv) post-translational modification of the protein product (e.g., using protein detection procedures described herein).

As used herein, the terms “target,” “targeting,” and “targeted,” in the context of the design of an siRNA, refers to generating an antisense strand so as to anneal the antisense strand to a region within the mRNA transcript of interest in a manner that results in a reduction in translation of the mRNA into the protein product.

As used herein, the terms “chemically modified nucleotide,” “nucleotide analog,” “altered nucleotide,” and “modified nucleotide” refer to a non-standard nucleotide, including non-naturally occurring ribonucleotides or deoxyribonucleotides. Exemplary nucleotide analogs are modified at any position so as to alter certain chemical properties of the nucleotide yet retain the ability of the nucleotide analog to perform its intended function.

As used herein, the term “metabolically stabilized” refers to RNA molecules that contain ribonucleotides that have been chemically modified in order to decrease the rate of metabolism of an RNA molecule that is administered to a subject. Exemplary modifications include 2′-hydroxy to 2′-O-methoxy or 2′-fluoro, and phosphodiester to phosphorothioate.

As used herein, the term “phosphorothioate” refers to a phosphate group of a nucleotide that is modified by substituting one or more of the oxygens of the phosphate group with sulfur.

As used herein, the terms “internucleoside linkage,” “internucleoside bond,” and the like refer to the bonds between nucleosides in a nucleic acid molecule.

As used herein, the term “antagomirs” refers to nucleic acids that can function as inhibitors of miRNA activity.

As used herein, the term “gapmers” refers to chimeric antisense nucleic acids that contain a central block of deoxynucleotide monomers sufficiently long to induce RNase H cleavage. The deoxynucleotide block is flanked by ribonucleotide monomers or ribonucleotide monomers containing modifications.

As used herein, the term “mixmers” refers to nucleic acids that contain a mix of locked nucleic acids (LNAs) and DNA.

As used herein, the term “guide RNAs” refers to nucleic acids that have sequence complementarity to a specific sequence in the genome immediately or 1 base pair upstream of the protospacer adjacent motif (PAM) sequence as used in CRISPR/Cas9 gene editing systems. Alternatively, “guide RNAs” may refer to nucleic acids that have sequence complementarity (e.g., are antisense) to a specific messenger RNA (mRNA) sequence. In this context, a guide RNA may also have sequence complementarity to a “passenger RNA” sequence of equal or shorter length, which is identical or substantially identical to the sequence of mRNA to which the guide RNA hybridizes.

As used herein, the term “branched siRNA” refers to a compound containing two or more double-stranded siRNA molecules covalently bound to one another. Branched siRNA molecules may be “di-branched,” also referred to herein as “di-siRNA,” wherein the siRNA molecule includes 2 siRNA molecules covalently bound to one another, e.g., by way of a linker. Branched siRNA molecules may be “tri-branched,” also referred to herein as “tri-siRNA,” wherein the siRNA molecule includes 3 siRNA molecules covalently bound to one another, e.g., by way of a linker. Branched siRNA molecules may be “tetra-branched,” also referred to herein as “tetra-siRNA,” wherein the siRNA molecule includes 4 siRNA molecules covalently bound to one another, e.g., by way of a linker.

As used herein, the term “branch point moiety” refers to a chemical moiety of a branched siRNA structure of the disclosure that may be covalently linked to a 5′ end or a 3′ end of an antisense strand or a sense strand of an siRNA molecule and which may support the attachment of additional single- or double-stranded siRNA molecules. Non-limiting examples of branch point moieties suitable for use in conjunction with the disclosed methods and compositions include, e.g., phosphoroamidite, tosylated solketal, 1,3-diaminopropanol, pentaerythritol, and any one of the branch point moieties described in U.S. Pat. No. 10,478,503.

The term “phosphate moiety” as used herein, refers to a terminal phosphate group that includes phosphates as well as modified phosphates. The phosphate moiety may be located at either terminus but is preferred at the 5′-terminal nucleoside. In one aspect, the terminal phosphate is unmodified having the formula —O—P(═O)(OH)OH. In another aspect, the terminal phosphate is modified such that one or more of the O and OH groups are replaced with H, O, S, N(R′) or alkyl where R′ is H, an amino protecting group or unsubstituted or substituted alkyl. In some embodiments, the 5′ and or 3′ terminal group may include from 1 to 3 phosphate moieties that are each, independently, unmodified (di- or tri-phosphates) or modified.

As used herein, the term “5′ phosphorus stabilizing moiety” refers to a terminal phosphate group that includes phosphates as well as modified phosphates (e.g., phosphorothioates, phosphodiesters, phosphonates). The phosphate moiety may be located at either terminus but is preferred at the 5′-terminal nucleoside. In one aspect, the terminal phosphate is unmodified having the formula —O—P(═O)(OH)OH. In another aspect, the terminal phosphate is modified such that one or more of the O and OH groups are replaced with H, O, S, N(R′), or alkyl where R′ is H, an amino protecting group, or unsubstituted or substituted alkyl. In some embodiments, the 5′ and or 3′ terminal group may include from 1 to 3 phosphate moieties that are each, independently, unmodified (di- or tri-phosphates) or modified.

The phosphate group of the nucleotide may also be modified, e.g., by substituting one or more of the oxygens of the phosphate group with sulfur (e.g., phosphorothioates), or by making other substitutions which allow the nucleotide to perform its intended function such as described in, for example, Eckstein, Antisense Nucleic Acid Drug Dev. 10:117-21, 2000; Rusckowski et al., Antisense Nucleic Acid Drug Dev. 10:333-45, 2000; Stein, Antisense Nucleic Acid Drug Dev. 11:317-25, 2001; Vorobjev et al., Antisense Nucleic Acid Drug Dev. 11:77-85, 2001; and U.S. Pat. No. 5,684,143. Certain of the above-referenced modifications (e.g., phosphate group modifications) preferably decrease the rate of hydrolysis of, for example, polynucleotides including said analogs in vivo or in vitro.

As used herein, the term “complementary” refers to two nucleotides that form canonical Watson-Crick base pairs. For the avoidance of doubt, Watson-Crick base pairs in the context of the present disclosure include adenine-thymine, adenine-uracil, and cytosine-guanine base pairs. A proper Watson-Crick base pair is referred to in this context as a “match,” while each unpaired nucleotide, and each incorrectly paired nucleotide, is referred to as a “mismatch.” Alignment for purposes of determining percent nucleic acid sequence complementarity can be achieved in various ways that are within the capabilities of one of skill in the art, for example, using publicly available computer software such as BLAST, BLAST-2, or Megalign software.

“Percent (%) sequence complementarity” with respect to a reference polynucleotide sequence is defined as the percentage of nucleic acids in a candidate sequence that are complementary to the nucleic acids in the reference polynucleotide sequence, after aligning the sequences and introducing gaps, if necessary, to achieve the maximum percent sequence complementarity. A given nucleotide is considered to be “complementary” to a reference nucleotide as described herein if the two nucleotides form canonical Watson-Crick base pairs. For the avoidance of doubt, Watson-Crick base pairs in the context of the present disclosure include adenine-thymine, adenine-uracil, and cytosine-guanine base pairs. A proper Watson-Crick base pair is referred to in this context as a “match,” while each unpaired nucleotide, and each incorrectly paired nucleotide, is referred to as a “mismatch.” Alignment for purposes of determining percent nucleic acid sequence complementarity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal complementarity over the full length of the sequences being compared. As an illustration, the percent sequence complementarity of a given nucleic acid sequence, A, to a given nucleic acid sequence, B, (which can alternatively be phrased as a given nucleic acid sequence, A that has a certain percent complementarity to a given nucleic acid sequence, B) is calculated as follows:

$$100 multiplied by ( the fraction X / Y )$$

where X is the number of complementary base pairs in an alignment (e.g., as executed by computer software, such as BLAST) in that program's alignment of A and B, and where Y is the total number of nucleic acids in B. It will be appreciated that where the length of nucleic acid sequence A is not equal to the length of nucleic acid sequence B, the percent sequence complementarity of A to B will not equal the percent sequence complementarity of B to A. As used herein, a query nucleic acid sequence is considered to be “completely complementary” to a reference nucleic acid sequence if the query nucleic acid sequence has 100% sequence complementarity to the reference nucleic acid sequence.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

$$100 multiplied by ( the fraction X / Y )$$

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

The term “complementarity sufficient to hybridize,” as used herein, refers to a nucleic acid sequence or a portion thereof that need not be fully complementary (e.g., 100% complementary) to a target region or a nucleic acid sequence or a portion thereof that has one or more nucleotide mismatches relative to the target region but that is still capable of hybridizing to the target region under specified conditions. For example, the nucleic acid may be, e.g., 95% complementary, 90%, complementary, 85% complementary, 80% complementary, 75% complementary, 70% complementary, 65% complementary, 60% complementary, 55% complementary, 50% complementary, or less, but still form sufficient base pairs with the target so as to hybridize across its length.

“Hybridization” or “annealing” of nucleic acids is achieved when one or more nucleoside residues within a polynucleotide base pairs with one or more complementary nucleosides to form a stable duplex. The base pairing is typically driven by hydrogen bonding events. Hybridization includes Watson-Crick base pairs formed from natural and/or modified nucleobases. The hybridization can also include non-Watson-Crick base pairs, such as wobble base pairs (guanosine-uracil, hypoxanthine-uracil, hypoxanthine-adenine, and hypoxanthine-cytosine) and Hoogsteen base pairs. Nucleic acids need not be 100% complementary to undergo hybridization. For example, one nucleic acid may be, e.g., 95% complementary, 90%, complementary, 85% complementary, 80% complementary, 75% complementary, 70% complementary, 65% complementary, 60% complementary, 55% complementary, 50% complementary, or less, relative to another nucleic acid, but the two nucleic acids may still form sufficient base pairs with one another so as to hybridize.

The “stable duplex” formed upon the annealing/hybridization of one nucleic acid to another is a duplex structure that is not denatured by a stringent wash. Exemplary stringent wash conditions are known in the art and include temperatures of about 5° C. less than the melting temperature of an individual strand of the duplex and low concentrations of monovalent salts, such as monovalent salt concentrations (e.g., NaCl concentrations) of less than 0.2 M (e.g., 0.2 M, 0.19 M, 0.18 M, 0.17 M, 0.16 M, 0.15 M, 0.14 M, 0.13 M, 0.12 M, 0.11 M) 0.1 M, 0.09 M, 0.08 M, 0.07 M, 0.06 M, 0.05 M, 0.04 M, 0.03 M, 0.02 M, 0.01 M, or less).

The term “gene silencing” refers to the suppression of gene expression, e.g., endogenous gene expression of a target gene, which may be mediated through processes that affect transcription and/or through processes that affect post-transcriptional mechanisms. In some embodiments, gene silencing occurs when an RNAi molecule initiates the inhibition or degradation of the mRNA transcribed from a gene of interest in a sequence-specific manner by way of RNA interference, thereby preventing translation of the gene's product.

The phrase “overactive disease driver gene,” as used herein, refers to a gene having increased activity and/or expression that contributes to or causes a disease state in a subject (e.g., a human). The disease state may be caused or exacerbated by the overactive disease driver gene directly or by way of an intermediate gene(s).

The term “negative regulator,” as used herein, refers to a gene that negatively regulates (e.g., reduces or inhibits) the expression and/or activity of another gene or set of genes (e.g., dysregulated gene or dysregulated gene pathway).

The term “positive regulator,” as used herein, refers to a gene that positively regulates (e.g., increases or saturates) the expression and/or activity of another gene or set of genes (e.g., dysregulated gene or dysregulated gene pathway).

As used herein, the term “ethylene glycol chain” refers to a carbon chain with the formula ((CH₂OH)₂).

As used herein, “alkyl” refers to a saturated hydrocarbon group. Alkyl groups may be acyclic or cyclic and contain only C and H when unsubstituted. When an alkyl residue having a specific number of carbons is named, all geometric isomers having that number of carbons are intended to be encompassed and described; thus, for example, “butyl” is meant to include n-butyl, sec-butyl, and iso-butyl. Examples of alkyl include ethyl, propyl, butyl, pentyl, hexyl, heptyl, octyl, cyclopropyl, cyclobutyl, cyclopentyl, cyclohexyl, cycloheptyl, cyclooctyl, and the like. In some embodiments, alkyl may be substituted. Suitable substituents that may be introduced into an alkyl group include, for example, hydroxy, alkoxy, amino, alkylamino, and halo, among others.

As used herein, “alkenyl” refers to an acyclic or cyclic unsaturated hydrocarbon group having at least one site of olefinic unsaturation (i.e., having at least one moiety of the formula C═C). Alkenyl groups contain only C and H when unsubstituted. When an alkenyl residue having a specific number of carbons is named, all geometric isomers having that number of carbons are intended to be encompassed and described; thus, for example, “butenyl” is meant to include n-butenyl, sec-butenyl, and iso-butenyl. Examples of alkenyl include —CH═CH₂, —CH₂—CH═CH₂, and —CH₂—CH═CH—CH═CH₂. In some embodiments, alkenyl may be substituted. Suitable substituents that may be introduced into an alkenyl group include, for example, hydroxy, alkoxy, amino, alkylamino, and halo, among others.

As used herein, “alkynyl” refers to an acyclic or cyclic unsaturated hydrocarbon group having at least one site of acetylenic unsaturation (i.e., having at least one moiety of the formula C≡C). Alkynyl groups contain only C and H when unsubstituted. When an alkynyl residue having a specific number of carbons is named, all geometric isomers having that number of carbons are intended to be encompassed and described; thus, for example, “pentynyl” is meant to include n-pentynyl, sec-pentynyl, iso-pentynyl, and tert-pentynyl. Examples of alkynyl include —C≡CH and —C≡C—CH₃. In some embodiments, alkynyl may be substituted. Suitable substituents that may be introduced into an alkynyl group include, for example, hydroxy, alkoxy, amino, alkylamino, and halo, among others.

As used herein the term “phenyl” denotes a monocyclic arene in which one hydrogen atom from a carbon atom of the ring has been removed. A phenyl group may be unsubstituted or substituted with one or more suitable substituents, wherein the substituent replaces an H of the phenyl group.

As used herein, the term “benzyl” refers to monovalent radical obtained when a hydrogen atom attached to the methyl group of toluene is removed. A benzyl group generally has the formula of phenyl-CH₂—. A benzyl group may be unsubstituted or substituted with one or more suitable substituents. For example, the substituent may replace an H of the phenyl component and/or an H of the methylene (—CH₂—) component.

As used herein, the term “amide” refers to an alkyl, alkenyl, alkynyl, or aromatic group that is attached to an amino-carbonyl functional group.

As used herein, the term “triazole” refers to heterocyclic compounds with the formula (C₂H₃N₃), having a five-membered ring of two carbons and three nitrogens, the positions of which can change resulting in multiple isomers.

As used herein, the term “terminal group” refers to the group at which a carbon chain or nucleic acid ends.

As used herein, an “amino acid” refers to a molecule containing amine and carboxyl functional groups and a side chain specific to the amino acid.

In some embodiments the amino acid is chosen from the group of proteinogenic amino acids. In some embodiments, the amino acid is an L-amino acid or a D-amino acid. In some embodiments, the amino acid is a synthetic amino acid (e.g., a beta-amino acid).

As used herein, the term “lipophilic amino acid” refers to an amino acid including a hydrophobic moiety (e.g., an alkyl chain or an aromatic ring).

As used herein, the term “target of delivery” refers to the organ or part of the body to which it is desired to deliver the branched oligonucleotide compositions.

As used herein, the term “between X and Y” is inclusive of the values of X and Y. For example, “between X and Y” refers to the range of values between the value of X and the value of Y, as well as the value of X and the value of Y.

As used herein, the terms “subject’ and “patient” are used interchangeably and refer to an organism, such as a mammal (e.g., a human), that is suffering from, or is at risk of, a disease, disorder, or condition, as determined by a qualified professional (e.g., a doctor or a nurse practitioner) with or without known in the art laboratory test(s) of sample(s) from the subject.

As used herein, the term “reference subject” refers to a healthy control subject of the same or similar, e.g., age, sex, geographical region, and/or education level as a subject treated with a composition of the disclosure. A healthy reference subject is one that does not suffer from a disease associated with expression of a dysregulated gene or a dysregulated gene pathway. Moreover, a healthy reference subject is one that does not suffer from a disease associated with altered (e.g., increased or decreased) expression and/or activity of a gene.

As used herein, the terms “neuroinflammatory disease” and “neuroinflammatory disorder” are used interchangeably to refer to any condition that is in some way caused by neuroinflammation. “Neuroinflammation” refers to a range of immune responses in the central nervous system (e.g., in microglia). Neuroinflammation may be brain-derived or result from a systemic inflammatory response.

As used herein, the terms “neurodegenerative disease” and “neurodegenerative disorder” are used interchangeably to refer to any condition that is in some way caused by a loss of function or death of cells of the central nervous system or peripheral nervous system. Exemplary neurodegenerative diseases are Alzheimer's disease, Huntington's disease, amyotrophic lateral sclerosis, frontotemporal dementia, and spinocerebellar ataxias.

As used herein, the terms “treat,” “treated,” and “treating” mean both therapeutic treatment and prophylactic or preventative measures wherein the object is to prevent, ameliorate, or slow down (lessen) an undesired physiological condition, disorder, or disease, or obtain beneficial or desired clinical results. Beneficial or desired clinical results include, but are not limited to, a reduction in a patient's reliance on pharmacological treatments; alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical, cognitive, or behavioral parameter, not necessarily discernible by the patient; or enhancement or improvement of condition, disorder, or disease. Treatment includes eliciting a clinically significant response without excessive levels of side effects. Treatment also includes prolonging survival as compared to expected survival if not receiving treatment.

As used herein, the terms “benefit” and “response” are used interchangeably in the context of a subject undergoing therapy for the treatment of a disease. For example, clinical benefits in the context of a subject administered an siRNA molecule or siRNA composition of the disclosure include, without limitation, a reduction in the duration and/or frequency of symptoms of the disease experienced by the subject, and/or; a reduction in disease-associated phenotypes, and/or; a reduction in wild type transcripts, mutant transcripts, variant transcripts, or overexpressed transcripts, and/or splice isoforms of transcripts of a target gene.

### BRIEF DESCRIPTION OF THE FIGURES

FIG. 1 shows the in vitro knockdown of PRNP with siRNA molecules of the disclosure that contained a fixed nucleobase sequence at the 3′ end of the antisense strand when compared to the fully sequence matched siRNA molecule. The siRNA molecules had sequences at the 3′ end of the antisense strand as set out in Table 3, below.

FIGS. 2A, 2B, and 2C are graphs demonstrating the ability of siRNA molecules of the disclosure containing a fixed nucleobase sequence at the 3′ end of the antisense strand to silence PRNP in vivo. The siRNA molecules had fixed regions at the 3′ end of the antisense strand as set out in Table 4, below. Each siRNA was administered at a 5 nmol (FIG. 2A), 1 nmol (FIG. 2B), and 0.2 nmol (FIG. 2C) dose.

FIGS. 3A and 3B are graphs demonstrating the ability of siRNA molecules of the disclosure containing a fixed nucleobase sequence at the 3′ end of the antisense strand to silence HPRT1 in vivo. The siRNA molecules had fixed regions at the 3′ end of the antisense strand as set out in Table 5, below. Each siRNA was administered at a 3 nmol (FIG. 3A) and 1 nmol (FIG. 3B) dose.

FIGS. 3C-3E are graphs demonstrating the dose-dependent nature of HPRT1 knockdown by siRNA molecules of the disclosure containing a fixed nucleobase sequence at the 3′ end of the antisense strand. The siRNA molecules had fixed regions at the 3′ end of the antisense strand as set out in Table 5, below. FIG. 3C shows all siRNA molecules in Table 5. FIG. 3D shows only siRNA molecules 11 and 12 in Table 5. FIG. 3D shows only siRNA molecules 11 and 15 in Table 5.

FIG. 4 is a graph showing the IC50 values (pM), % mRNA expression, and goodness of fit (R²) for all 64 possible fixed nucleobase sequences of the three terminal nucleobases at the antisense strands of siRNA molecules targeting HPRT1.

### DETAILED DESCRIPTION

The present invention provides short-interfering RNA (siRNA) molecules, including single- and double-stranded short interfering RNA (ds-siRNA), and methods for their use in treating a patient in need of gene silencing (e.g., a patient having dysregulated gene expression, such as a patient with, e.g., Alzheimer's disease, amyotrophic lateral sclerosis, Parkinson's disease, frontotemporal dementia, Huntington's disease, multiple sclerosis, or progressive supranuclear palsy). siRNA molecules are capable of mediating RNA interference (RNAi) by degrading mRNA with a complementary nucleotide sequence, thus reducing, or altogether preventing, the translation of the target gene.

The siRNA molecules of the disclosure may contain a fixed sequence at the end of the sense strand or the antisense strand (e.g., at the 5′ end of the sense strand, the 5′ end of the antisense strand, the 3′ end of the sense strand, or the 3′ end of the antisense strand). The fixed sequence may be contained within an overhang portion of either strand (e.g., an overhang region of the sense strand that extends beyond the antisense strand or an overhang region of the antisense strand that extends beyond the sense strand).

The siRNA molecules described herein may employ a variety of chemical modifications. For example, the siRNA molecules described herein may include specific patterns of chemical modifications (e.g., 2′ ribose modifications or internucleoside linkage modifications) to improve resistance against nuclease enzymes, toxicity profile, and physicochemical properties (e.g., thermostability).

The siRNA molecules of the disclosure may feature an antisense strand having a nucleic acid sequence that is complementary to a region of an mRNA transcript in a target gene. The degree of complementarity of the antisense strand to the region of the target mRNA transcript may be sufficient for the antisense strand to anneal over the full length of the region of the mRNA transcript. For example, the antisense strand may have a nucleic acid sequence that is at least 60% complementary (e.g., 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementary) to the region of the target mRNA transcript.

### Fixed Nucleobase Regions

In some embodiments of the siRNA molecules of the disclosure, the antisense strand and/or the sense strand contains a region of nucleotides with a fixed sequence of nucleobases. In some embodiments, the region of fixed nucleotides is contained within the sense strand. In some embodiments, the region of fixed nucleotides is contained within the antisense strand. In some embodiments, the region of fixed nucleotides is part of the antisense strand and contains one or more mismatches relative to the target mRNA transcript. In some embodiments, the region of fixed nucleotides has a sequence that is independent of the sequence of the target mRNA transcript.

Without being bound by theory, certain fixed regions may impart a formed mRNA cleavage product with an increased off-rate from a RISC complex following cleavage of the target mRNA. Alternatively, or in addition, certain fixed regions may impart the antisense strand with an increased binding affinity for an endogenous Argonaute (AGO) protein relative to a corresponding antisense strand that is fully complementary to the target mRNA.

The present disclosure is based, at least in part, on the surprising discovery that installing fixed nucleobase regions (e.g., at the 3′ end of the antisense strand and/or in a region of the antisense strand overhanging the sense strand) that are independent of the target mRNA transcript and/or have one or more (e.g., from 1 to 4) nucleotide mismatches relative to the mRNA transcript impart surprising benefits on siRNA molecules. The fixed nucleobase regions of the disclosure effectuate surprising results in a manner that depends on a variety of factors, including, e.g., the precise number of mismatches in the tail, as well as the nature and location of chemical modifications in the tail. The length of the fixed nucleobase region has surprisingly been discovered to be an important factor, and the benefit imparted by installing the fixed nucleobase region may be affected by varying the length of the fixed nucleobase region. The sections that follow describes these and other parameters in further detail.

In some embodiments, certain fixed nucleobase sequences (e.g., at the 3′ end of the antisense strand and/or in a 3′ region of the antisense strand overhanging the sense strand) exhibit decreased neurotoxicity when administered to the CNS as compared to the corresponding siRNA that is fully complementary to a target mRNA transcript. In some embodiments, certain fixed nucleobase sequences (e.g., at the 3′ end of the antisense strand and/or in a 3′ region of the antisense strand overhanging the sense strand) have no observed acute neurotoxicity upon administration to the CNS.

In some embodiments, the fixed nucleobase region has from 1 to 4 (e.g., 1, 2 3, or 4) nucleotide mismatches relative to the target mRNA transcript. In some embodiments, the fixed nucleobase region has 1 nucleotide mismatch relative to the target mRNA transcript. In some embodiments, the fixed nucleobase region has 2 nucleotide mismatches relative to the target mRNA transcript. In some embodiments, the fixed nucleobase region has 3 nucleotide mismatches relative to the target mRNA transcript. In some embodiments, the fixed nucleobase region has 4 nucleotide mismatches relative to the target mRNA transcript. In some embodiments, the fixed nucleobase region has from 1 to 3 nucleotide mismatches relative to the target mRNA transcript. In some embodiments, the fixed nucleobase region has from 2 to 4 nucleotide mismatches relative to the target mRNA transcript. In some embodiments, the fixed nucleobase region has 3 or 4 nucleotide mismatches relative to the target mRNA transcript. In some embodiments, the fixed nucleobase region has 1 or 2 nucleotide mismatches relative to the target mRNA transcript. In some embodiments, the fixed nucleobase region has 2 or 3 nucleotide mismatches relative to the target mRNA transcript.

Mismatches at the 3′ end of the antisense strand may improve RISC-mediated target silencing, as complete complementarity between a RISC-loaded antisense strand and a target mRNA may destabilize the RISC complex and/or elicit ubiquitin ligase mediated degradation of the RISC complex.

In some embodiments, the 3′ end of the antisense strand has from 1 to 4 (e.g., 1, 2 3, or 4) nucleotide mismatches relative to the target mRNA transcript. In some embodiments, the 3′ end of the antisense strand has 1 nucleotide mismatch relative to the target mRNA transcript. In some embodiments, the 3′ end of the antisense strand has 2 nucleotide mismatches relative to the target mRNA transcript. In some embodiments, the 3′ end of the antisense strand has 3 nucleotide mismatches relative to the target mRNA transcript. In some embodiments, the 3′ end of the antisense strand has 4 nucleotide mismatches relative to the target mRNA transcript. In some embodiments, the 3′ end of the antisense strand has from 1 to 3 nucleotide mismatches relative to the target mRNA transcript. In some embodiments, the 3′ end of the antisense strand has from 2 to 4 nucleotide mismatches relative to the target mRNA transcript. In some embodiments, the 3′ end of the antisense strand has 3 or 4 nucleotide mismatches relative to the target mRNA transcript. In some embodiments, the 3′ end of the antisense strand has 1 or 2 nucleotide mismatches relative to the target mRNA transcript. In some embodiments, the 3′ end of the antisense strand has 2 or 3 nucleotide mismatches relative to the target mRNA transcript.

In some embodiments, the siRNA molecule may contain one or more regions as described above at the 5′ end of the sense strand. In some embodiments, the siRNA molecule may contain one or more regions as described above at the 5′ end of the antisense strand. In some embodiments, the siRNA molecule may contain one or more regions as described above at the 3′ end of the sense strand. In some embodiments, the siRNA molecule may contain one or more regions as described above at the 3′ end of the antisense strand. In some embodiments, the fixed region is contained within a region of the sense strand that overhangs the antisense strand. In some embodiments, the fixed region is contained within a region of the antisense strand that overhangs the sense strand.

In some embodiments, the fixed region is 1 nucleotide in length. In some embodiments, the fixed region is 2 nucleotides in length. In some embodiments, the fixed region is 3 nucleotides in length. In some embodiments, the fixed region is 4 nucleotides in length. In some embodiments, the fixed region is 5 nucleotides in length. In some embodiments, the fixed region is 6 nucleotides in length. In some embodiments, the fixed region is 7 nucleotides in length. In some embodiments, the fixed region is 8 nucleotides in length. In some embodiments, the fixed region is 9 nucleotides in length. In some embodiments, the fixed region is 10 nucleotides in length.

The fixed may be a region of contiguous or linked nucleotides, though it need not be. For example, the fixed region may be a region of 1 to 10 (e.g., 1, 2, 3, 4, 5, 6, 7, 8, 9, or 10) contiguous nucleotides with a sequence independent of the target mRNA transcript. Alternatively, there may be sequence matched nucleotides (that is, nucleotides that are complementary to the target mRNA transcript) intervening with nucleotides that are independent of the target mRNA transcript. For example, there may be one or more mismatched nucleotides, followed by one or more sequence matched nucleotides, followed by one or more mismatched nucleotides, or any other permutation of matched and mismatched nucleotides, without affecting the ability of the siRNA molecule to carry out gene silencing.

In some embodiments, the fixed region is 3 nucleotides in length and is at the 3′ end of the antisense strand in a region overhanging the sense strand. In some embodiments, the fixed region has a nucleotide sequence selected from the sequences of Table 1a, below.

TABLE 1a
Exemplary Fixed Nucleobase Sequences

| | | | | | | | |
|---|---|---|---|---|---|---|---|
| AAA | AGA | CAA | CGA | GAA | GGA | UAA | UGA |
| AAC | AGC | CAC | CGC | GAC | GGC | UAC | UGC |
| AAG | AGG | CAG | CGG | GAG | GGG | UAG | UGG |
| AAU | AGU | CAU | CGU | GAU | GGU | UAU | UGU |
| ACA | AUA | CCA | CUA | GCA | GUA | UCA | UUA |
| ACC | AUC | CCC | CUC | GCC | GUC | UCC | UUC |
| ACG | AUG | CCG | CUG | GCG | GUG | UCG | UUG |
| ACU | AUU | CCU | CUU | GCU | GUU | UCU | UUU |

In some embodiments, the fixed region is 4 nucleotides in length and is at the 3′ end of the antisense strand in a region overhanging the sense strand. In some embodiments, the fixed region has a nucleotide sequence selected from the sequences of Table 1b, below.

TABLE 1b
Exemplary Fixed Nucleobase Sequences

| | | | | | | | |
|---|---|---|---|---|---|---|---|
| AAAA | UAAA | GAAA | CAAA | AGAA | UGAA | GGAA | CGAA |
| AAAC | UAAC | GAAC | CAAC | AGAC | UGAC | GGAC | CGAC |
| AAAG | UAAG | GAAG | CAAG | AGAG | UGAG | GGAG | CGAG |
| AAAU | UAAU | GAAU | CAAU | AGAU | UGAU | GGAU | CGAU |
| AACA | UACA | GACA | CACA | AGCA | UGCA | GGCA | CGCA |
| AACC | UACC | GACC | CACC | AGCC | UGCC | GGCC | CGCC |
| AACG | UACG | GACG | CACG | AGCG | UGCG | GGCG | CGCG |
| AACU | UACU | GACU | CACU | AGCU | UGCU | GGCU | CGCU |
| AAGA | UAGA | GAGA | CAGA | AGGA | UGGA | GGGA | CGGA |
| AAGC | UAGC | GAGC | CAGC | AGGC | UGGC | GGGC | CGGC |
| AAGG | UAGG | GAGG | CAGG | AGGG | UGGG | GGGG | CGGG |
| AAGU | UAGU | GAGU | CAGU | AGGU | UGGU | GGGU | CGGU |
| AAUA | UAUA | GAUA | CAUA | AGUA | UGUA | GGUA | CGUA |
| AAUC | UAUC | GAUC | CAUC | AGUC | UGUC | GGUC | CGUC |
| AAUG | UAUG | GAUG | CAUG | AGUG | UGUG | GGUG | CGUG |
| AAUU | UAUU | GAUU | CAUU | AGUU | UGUU | GGUU | CGUU |
| ACAA | UCAA | GCAA | CCAA | AUAA | UUAA | GUAA | CUAA |
| ACAC | UCAC | GCAC | CCAC | AUAC | UUAC | GUAC | CUAC |
| ACAG | UCAG | GCAG | CCAG | AUAG | UUAG | GUAG | CUAG |
| ACAU | UCAU | GCAU | CCAU | AUAU | UUAU | GUAU | CUAU |
| ACCA | UCCA | GCCA | CCCA | AUCA | UUCA | GUCA | CUCA |
| ACCC | UCCC | GCCC | CCCC | AUCC | UUCC | GUCC | CUCC |
| ACCG | UCCG | GCCG | CCCG | AUCG | UUCG | GUCG | CUCG |
| ACCU | UCCU | GCCU | CCCU | AUCU | UUCU | GUCU | CUCU |
| ACGA | UCGA | GCGA | CCGA | AUGA | UUGA | GUGA | CUGA |
| ACGC | UCGC | GCGC | CCGC | AUGC | UUGC | GUGC | CUGC |
| ACGG | UCGG | GCGG | CCGG | AUGG | UUGG | GUGG | CUGG |
| ACGU | UCGU | GCGU | CCGU | AUGU | UUGU | GUGU | CUGU |
| ACUA | UCUA | GCUA | CCUA | AUUA | UUUA | GUUA | CUUA |
| ACUC | UCUC | GCUC | CCUC | AUUC | UUUC | GUUC | CUUC |
| ACUG | UCUG | GCUG | CCUG | AUUG | UUUG | GUUG | CUUG |
| ACUU | UCUU | GCUU | CCUU | AUUU | UUUU | GUUU | CUUU |

In some embodiments, the fixed regions described herein may have any combination of modifications (e.g., 2′ sugar modifications, internucleoside linkage modifications, modified nucleobases, and/or any of the modified intersubunit linkages of Formula E1 and subformula thereof).

In some embodiments of any of the fixed regions described herein, the fixed region includes at least one (e.g., 1, 2, 3, 4, 5, or more) modified internucleoside linkage.

In some embodiments, at least one modified intersubunit linkage is of Formula E1:

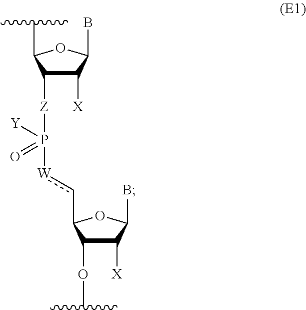

wherein:
   each B is, independently, a base pairing moiety;
   W is selected from the group consisting of O, S, B, BR², N, NR², OCH₂, OCH, CH₂, and CH, optionally wherein W is selected from the group consisting of OCH₂ and OCH;
   each X is, independently, selected from the group consisting of halo (e.g., fluoro or chloro), hydroxy, and C₁₋₆ alkoxy, optionally wherein each X is, independently, selected from the group consisting of halo (e.g., fluoro or chloro) and C₁₋₆ alkoxy (e.g., methoxy, ethoxy, 2-methoxyethoxy, n-propoxy, sec-propoxy, n-butoxy, sec-butoxy, tert-butoxy, n-pentoxy, or n-heptoxy);
   Y is selected from the group consisting of O⁻, OH, OR, NH⁻, NH₂, S⁻, and SH, optionally wherein Y is selected from the group consisting of O⁻, OH, and OR.
Z is selected from the group consisting of O, S, BR², NR², and CH₂;
R is a protecting group;
each R² is, independently, H or optionally substituted C₁-C₆ alkyl; and
[symbol] is an optional double bond.

In some embodiments of Formula E1, W is OCH₂.

In some embodiments of Formula E1, W is OCH and [symbol] is a double bond.

In some embodiments of Formula E1, Z is O.

In some embodiments of Formula E1, Z is CH₂.

In some embodiments of Formula E1, when Y is O⁻, either Z or W is not O.

In some embodiments of Formula E1, Z is CH₂ and W is CH₂. In some embodiments, the modified intersubunit linkage of Formula E1 is a modified intersubunit linkage of Formula E2:

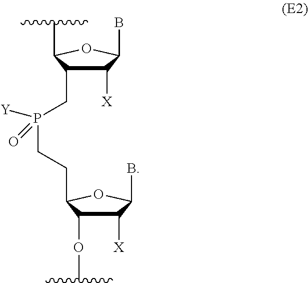

In some embodiments of Formula E1, Z is CH₂ and W is O. In some embodiments, the modified intersubunit linkage of Formula E1 is a modified intersubunit linkage of Formula E3:

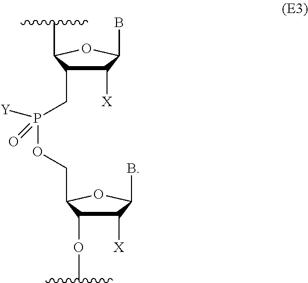

In some embodiments of Formula E1, Z is O and W is CH₂. In some embodiments, the modified intersubunit linkage of Formula E1 is a modified intersubunit linkage of Formula E4:

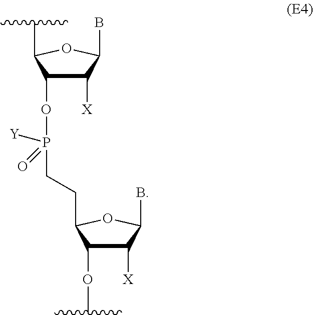

In some embodiments of Formula E1, Z is O and W is CH. In some embodiments, the modified intersubunit linkage of Formula E1 is a modified intersubunit linkage of Formula E5:

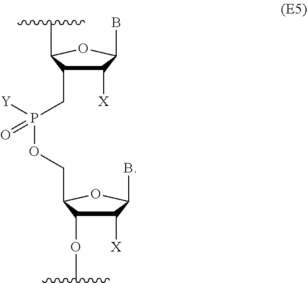

In some embodiments, the modified intersubunit linkage of Formula E1 is a modified intersubunit linkage of Formula E6:

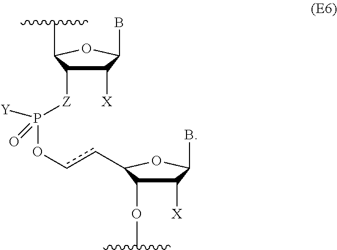

In some embodiments of Formula E6:
each B is, independently, a base pairing moiety;
   each X is, independently, selected from the group consisting of halo, hydroxy, and C₁₋₆ alkoxy; optionally wherein each X is, independently, selected from the group consisting of halo (e.g., fluoro) and C₁₋₆ alkoxy (e.g., methoxy, ethoxy, n-propoxy, sec-propoxy, n-butoxy, sec-butoxy, tert-butoxy, n-pentoxy, or n-heptoxy);
   Y is selected from the group consisting of O⁻, OH, OR, NH⁻, NH₂, S⁻, and SH, optionally wherein Y is selected from the group consisting of O⁻, OH, and OR;
Z is selected from the group consisting of O and CH₂; and
[symbol] is an optional double bond.

In some embodiments of Formula E6:
each X is, independently, selected from the group consisting of fluoro, hydroxy, and C₁₋₆ alkoxy; optionally wherein each X is, independently, selected from the group consisting of fluoro and C₁₋₆ alkoxy (e.g., methoxy, ethoxy, n-propoxy, sec-propoxy, n-butoxy, sec-butoxy, tert-butoxy, n-pentoxy, or n-heptoxy);
Y is selected from the group consisting of O⁻, OH, and OR;
Z is selected from the group consisting of O and CH₂; and
[symbol] is an optional double bond.

In some embodiments of Formula E6:
each X is, independently, selected from the group consisting of fluoro, hydroxy, methoxy, ethoxy, n-propoxy, sec-propoxy, n-butoxy, sec-butoxy, tert-butoxy, n-pentoxy, and n-heptoxy;
Y is selected from the group consisting of O⁻, OH, and OR;
Z is selected from the group consisting of O and CH₂; and
[symbol] is an optional double bond.

In some embodiments of Formula E6:
each X is, independently, selected from the group consisting of fluoro, hydroxy, methoxy, ethoxy, n-propoxy, sec-propoxy, n-butoxy, sec-butoxy, tert-butoxy, n-pentoxy, and n-heptoxy;
Y is selected from the group consisting of O⁻, OH, and OR;
Z is O; and
[symbol] is an optional double bond.

In some embodiments of Formula E6:
each X is, independently, selected from the group consisting of fluoro, hydroxy, methoxy, ethoxy, n-propoxy, sec-propoxy, n-butoxy, sec-butoxy, tert-butoxy, n-pentoxy, and n-heptoxy;
Y is selected from the group consisting of O⁻, OH, and OR;
Z is CH₂; and
[symbol] is an optional double bond.

In some embodiments of Formula E6:
each X is, independently, selected from the group consisting of fluoro, hydroxy, and methoxy;
Y is selected from the group consisting of O⁻, OH, and OR;
Z is O; and
[symbol] is an optional double bond.

In some embodiments of Formula E6:
each X is, independently, selected from the group consisting of fluoro, hydroxy, and methoxy;
Y is selected from the group consisting of O⁻, OH, and OR;
Z is CH₂; and
[symbol] is an optional double bond.

In some embodiments of Formula E1, Z is O and W is OCH₂. In some embodiments, the modified intersubunit linkage of Formula E1 is a modified intersubunit linkage of Formula E6a:

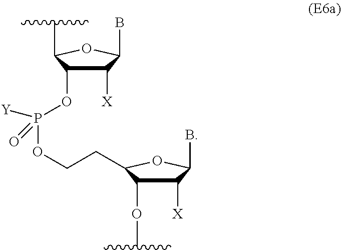

In some embodiments of Formula E1, Z is CH₂ and W is CH. In some embodiments, the modified intersubunit linkage of Formula E1 is a modified intersubunit linkage of Formula E7:

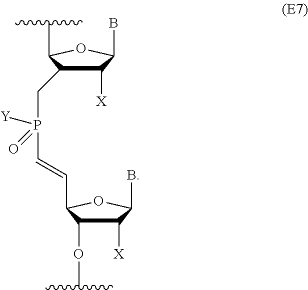

In some embodiments of Formula E1, the base pairing moiety B is selected from the group consisting of adenine, guanine, cytosine, and uracil.

In some embodiments, at least one modified intersubunit linkage is of Formula E8:

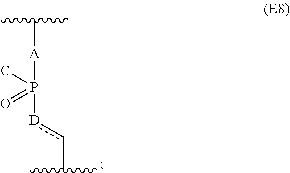

wherein:
   D is selected from the group consisting of O, S, B, BR², N, NR², OCH₂, OCH, CH₂, and CH, optionally wherein D is selected from the group consisting of OCH₂ and OCH;
   C is selected from the group consisting of O⁻, OH, OR¹, NH⁻, NH₂, S⁻, and SH, optionally wherein C is selected from the group consisting of O⁻, OH, and OR¹;
   A is selected from the group consisting of O, S, BR², NR² and CH₂;
   R¹ is a protecting group;
   each R² is, independently, H or optionally substituted C₁-C₆ alkyl;
   [symbol] is an optional double bond; and
   the intersubunit is bridging two optionally modified nucleosides.

In some embodiments, D is OCH₂.

In some embodiments, D is OCH and [symbol] is a double bond.

In some embodiments, A is O.

In some embodiments, A is CH₂.

In some embodiments, when C is O⁻, either A or D is not O.

In some embodiments, D is CH₂. In another embodiment, the modified intersubunit linkage of Formula E8 is a modified intersubunit linkage of Formula E9.

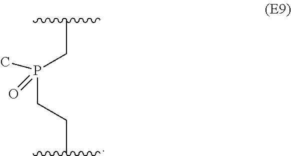 [SMILES: *CCP(C)(=O)C*]

In some embodiments, D is O. In another embodiment, the modified intersubunit linkage of Formula E8 is a modified intersubunit linkage of Formula E10:

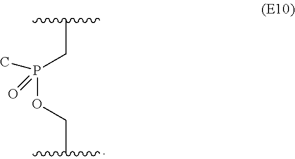 [SMILES: *COP(C)(=O)C*]

In some embodiments, D is CH₂. In another embodiment, the modified intersubunit linkage of Formula E8 is a modified intersubunit linkage of Formula E11:

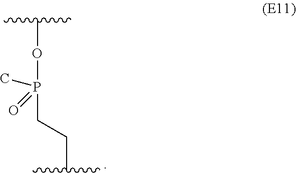 [SMILES: *CCP(C)(=O)O*]

In some embodiments, D is CH. In another embodiment, the modified intersubunit linkage of Formula E8 is a modified intersubunit linkage of Formula E12:

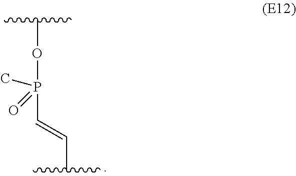 [SMILES: */C=C/P(C)(=O)O*]

In some embodiments, D is OCH₂. In another embodiment, the modified intersubunit linkage of Formula E8 is a modified intersubunit linkage of Formula E13:

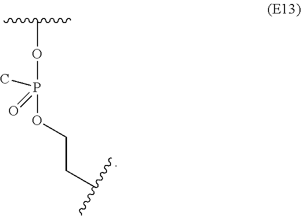 [SMILES: *CCOP(C)(=O)O*]

In another embodiment, the modified intersubunit linkage of Formula E8 is a modified intersubunit linkage of Formula E14:

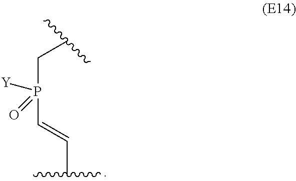

In another embodiment, the modified intersubunit linkage of Formula E8 is a modified intersubunit linkage of Formula E15:

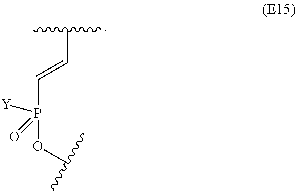

In some embodiments of the modified siRNA linkage, each optionally modified nucleoside is independently, at each occurrence, selected from the group consisting of adenosine, guanosine, cytidine, and uridine.

In some embodiments, at least one modified intersubunit linkage is of Formula E8:

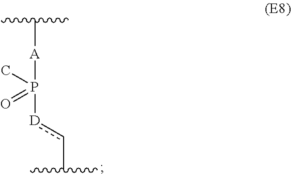

wherein:
   D is selected from the group consisting of O, OCH₂, OCH, CH₂, and CH, optionally wherein D is selected from the group consisting of OCH₂ and OCH;
   C is selected from the group consisting of O⁻, OH, OR¹, NH⁻, NH₂, S⁻, and SH, optionally wherein C is selected from the group consisting of O⁻, OH, and OR¹;
   A is selected from the group consisting of O and CH₂;
R1 is a protecting group selected from the group consisting of dimethoxytrityl (DMTr), succinate, tert-butyl dimethylsilyl (TBDMS), benzoyl (Bz), benzyl (Bn), methoxyethoxymethyl ether (MOM), methoxybenzyl ether (PMB), methylthiomethyl ether, pivaloyl (Piv), tetrahydropyranyl (THP), tetrahydrofuranyl (THF), trityl (Trt), triisopropylsilyl (TIPS), tert-butyldiphenylsilyl (TBDPS), and acetate;
[symbol] is an optional double bond;
and the intersubunit is bridging two optionally modified nucleosides.

In some embodiments, D is OCH₂.

In some embodiments, D is OCH and [symbol] is a double bond.

In some embodiments, A is O.

In some embodiments, A is CH₂.

In some embodiments of any of the modified intersubunit linkages described above, the base pairing moiety B is selected from the group consisting of adenine, guanine, cytosine, and uracil.

In some embodiments, R is a protecting group selected from the group consisting of dimethoxytrityl (DMTr), succinate, tert-butyl dimethylsilyl (TBDMS), benzoyl (Bz), benzyl (Bn), methoxyethoxymethyl ether (MOM), methoxybenzyl ether (PMB), methylthiomethyl ether, pivaloyl (Piv), tetrahydropyranyl (THP), tetrahydrofuranyl (THF), trityl (Trt), triisopropylsilyl (TIPS), tert-butyldiphenylsilyl (TBDPS), and acetate; and
[symbol] is an optional double bond.

In some embodiments, the fixed region contains at least one (e.g., 1, 2, 3, 4, 5, or more) phosphorothioate internucleoside linkage.

In some embodiments, the fixed region contains at least one (e.g., 1, 2, 3, 4, 5, or more) nucleotides containing a modified ribose. In some embodiments, the fixed region contains at least one (e.g., 1, 2, 3, 4, 5, or more) 2′-methoxy nucleotides. In some embodiments, the fixed region contains at least one (e.g., 1, 2, 3, 4, 5, or more) 2′-fluoro nucleotides.

In some embodiments, the fixed region is three nucleotides in length at the 3′ end of the antisense strand, optionally wherein the fixed region is within a portion of the antisense strand that overhangs the sense strand. In some embodiments, the fixed region has any one of Formula F1-F6:

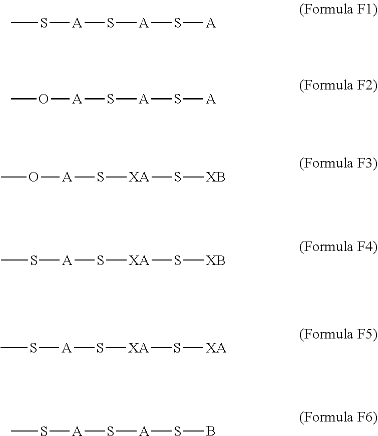

wherein S is a phosphorothioate internucleoside linkage, O is a phosphodiester internucleoside linkage, A is a 2′-methoxy nucleotide, B is a 2′-fluoro nucleotide, XA is a 2′-methoxy nucleotide of Formula E6a, and XB is a 2′-fluoro nucleotide of Formula E6a.

In some embodiments, the fixed region has any of the following patterns:

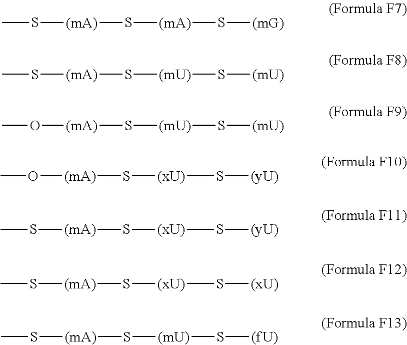

wherein S is a phosphorothioate internucleoside linkage, O is a phosphodiester internucleoside linkage, mA is a 2′-methoxyadenosine, mG is a 2′-methoxy guanidine, mU is a 2′-methoxyuridine, xU is a 2′methoxyuridine of Formula E6a, and yU is a 2′-fluorouridine of Formula E6a.
   siRNA Structure

The siRNA molecules of the disclosure may be in the form of a single-stranded (ss) or double-stranded (ds) oligonucleotide structure. In some embodiments, the siRNA molecules may be di-branched, tri-branched, or tetra-branched molecules. Furthermore, the siRNA molecules of the disclosure may contain one or more phosphodiester internucleoside linkages and/or an analog thereof, such as a phosphorothioate internucleoside linkage. The siRNA molecules of the disclosure may further contain chemically modified nucleosides having 2′ sugar modifications.

The simplest siRNAs consist of a ribonucleic acid, including a ss- or ds-structure, formed by a first strand (i.e., antisense strand), and in the case of a ds-siRNA, a second strand (i.e., sense strand). The first strand includes a stretch of contiguous nucleotides that is at least partially complementary to a target nucleic acid. The second strand also includes a stretch of contiguous nucleotides where the second stretch is at least partially identical to a target nucleic acid. The first strand and said second strand may be hybridized to each other to form a double-stranded structure. The hybridization typically occurs by Watson Crick base pairing.

Depending on the sequence of the first and second strand, the hybridization or base pairing is not necessarily complete or perfect, which means that the first and second strand are not 100% base-paired due to mismatches. One or more mismatches may also be present within the duplex without necessarily impacting the siRNA RNAi activity.

The first strand contains a stretch of contiguous nucleotides which is essentially complementary to a target nucleic acid. Typically, the target nucleic acid sequence is, in accordance with the mode of action of interfering ribonucleic acids, a ss-RNA, preferably an mRNA. Such hybridization occurs most likely through Watson Crick base pairing but is not necessarily limited thereto. The extent to which the first strand has a complementary stretch of contiguous nucleotides to a target nucleic acid sequence may be between 80% and 100%, e.g., 80%, 85%, 90%, 95%, or 100% complementary.

The siRNA molecules described herein may employ modifications to the nucleobase, phosphate backbone, ribose core, 5′- and 3′-ends, and branching, wherein multiple strands of siRNA may be covalently linked.

### Lengths of Small Interfering RNA Molecules

It is within the scope of the disclosure that any length, known and previously unknown in the art, may be employed for the current invention. As described herein, potential lengths for an antisense strand of the siRNA molecules of the present disclosure is between 10 and 30 nucleotides (e.g., 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides), 15 and 25 nucleotides (e.g., 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, or 25 nucleotides), or 18 and 23 nucleotides (e.g., 18 nucleotides, 19 nucleotides, 20 nucleotides, 21 nucleotides, 22 nucleotides, or 23 nucleotides). In some embodiments, the antisense strand is 20 nucleotides. In some embodiments, the antisense strand is 21 nucleotides. In some embodiments, the antisense strand is 22 nucleotides. In some embodiments, the antisense strand is 23 nucleotides. In some embodiments, the antisense strand is 24 nucleotides. In some embodiments, the antisense strand is 25 nucleotides. In some embodiments, the antisense strand is 26 nucleotides. In some embodiments, the antisense strand is 27 nucleotides. In some embodiments, the antisense strand is 28 nucleotides. In some embodiments, the antisense strand is 29 nucleotides. In some embodiments, the antisense strand is 30 nucleotides.

In some embodiments, the sense strand of the siRNA molecules of the present disclosure is between 12 and 30 nucleotides (e.g., 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides), or 14 and 23 nucleotides (e.g., 14 nucleotides, 15 nucleotides, 16 nucleotides, 17 nucleotides, 18 nucleotides, 19 nucleotides, 20 nucleotides, 21 nucleotides, 22 nucleotides, or 23 nucleotides). In some embodiments, the sense strand is 15 nucleotides. In some embodiments, the sense strand is 16 nucleotides. In some embodiments, the sense strand is 17 nucleotides. In some embodiments, the sense strand is 18 nucleotides. In some embodiments, the sense strand is 19 nucleotides. In some embodiments, the sense strand is 20 nucleotides. In some embodiments, the sense strand is 21 nucleotides. In some embodiments, the sense strand is 22 nucleotides. In some embodiments, the sense strand is 23 nucleotides. In some embodiments, the sense strand is 24 nucleotides. In some embodiments, the sense strand is 25 nucleotides. In some embodiments, the sense strand is 26 nucleotides. In some embodiments, the sense strand is 27 nucleotides. In some embodiments, the sense strand is 28 nucleotides. In some embodiments, the sense strand is 29 nucleotides. In some embodiments, the sense strand is 30 nucleotides.

### 2′ Sugar Modifications

The present disclosure may include ss- and ds-siRNA molecule compositions including at least one (e.g., at least 1, 2, 3, 4, 5, 6, 7, 8, 9, 10, 11, or more) nucleosides having 2′ sugar modifications. Possible 2′-modifications include all possible orientations of OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C1 to C10 alkyl or C2 to C10 alkenyl and alkynyl. In some embodiments, the modification includes a 2′-O-methyl(2′-O-Me) modification. Other potential sugar substituent groups include: C1 to C10 lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, a group for improving the pharmacokinetic properties of an oligonucleotide, or a group for improving the pharmacodynamic properties of an oligonucleotide, and other substituents having similar properties. In some embodiments, the modification includes 2′-methoxyethoxy (2′-O—CH₂CH₂OCH₃, also known as 2′-O-(2-methoxyethyl) or 2′-MOE). In some embodiments, the modification includes 2′-dimethylaminooxyethoxy, i.e., a O(CH₂)₂ON(CH₃)₂ group, also known as 2′-DMAOE, and 2′-dimethylaminoethoxyethoxy (also known in the art as 2′-O-dimethylamino-ethoxy-ethyl or 2′-DMAEOE), i.e., 2′-O—CH₂OCH₂N(CH₃)₂. Other potential sugar substituent groups include, e.g., aminopropoxy (—OCH₂CH₂CH₂NH₂), allyl (—CH₂—CH═CH₂), —O-allyl (—O—CH₂—CH═CH₂) and fluoro (F). 2′-sugar substituent groups may be in the arabino (up) position or ribo (down) position. In some embodiments, the 2′-arabino modification is 2′-F. Similar modifications may also be made at other positions on the siRNA molecule, particularly the 3′ position of the sugar on the 3′ terminal nucleoside or in 2′-5′ linked oligonucleotides and the 5′ position of 5′ terminal nucleotide. Oligonucleotides may also have sugar mimetics such as cyclobutyl moieties in place of the pentofuranosyl sugar.

### Nucleobase Modifications

The siRNA molecules of the disclosure may also include nucleosides or other surrogate or mimetic monomeric subunits that include a nucleobase (often referred to in the art simply as “base” or “heterocyclic base moiety”). The nucleobase is another moiety that has been extensively modified or substituted and such modified and or substituted nucleobases are amenable to the present disclosure. As used herein, “unmodified” or “natural” nucleobases include the purine bases adenine (A) and guanine (G), and the pyrimidine bases thymine (T), cytosine (C) and uracil (U). Modified nucleobases also referred herein as heterocyclic base moieties include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in Kroschwitz, J. I., ed. The Concise Encyclopedia of Polymer Science and Engineering, New York, John Wiley & Sons, 1990, pp. 858-859; those disclosed by Englisch et al., Angewandte Chemie, International Edition 30:613, 1991; and those disclosed by Sanghvi, Y. S., Chapter 16, Antisense Research and Applications, CRC Press, Gait, M. J. ed., 1993, pp. 289-302. The siRNA molecules of the present disclosure may also include polycyclic heterocyclic compounds in place of one or more heterocyclic base moieties. A number of tricyclic heterocyclic compounds have been previously reported. These compounds are routinely used in antisense applications to increase the binding properties of the modified strand to a target strand.

Representative cytosine analogs that make three hydrogen bonds with a guanosine in a second strand include 1,3-diazaphenoxazine-2-one (Kurchavov et al., Nucleosides and Nucleotides, 16:1837-46, 1997), 1,3-diazaphenothiazine-2-one (Lin et al. Am. Chem. Soc., 117:3873-4, 1995), and 6,7,8,9-tetrafluoro-1,3-diazaphenoxazine-2-one (Wang et al., Tetrahedron Lett., 39:8385-8, 1998). Incorporated into oligonucleotides, these base modifications were shown to hybridize with complementary guanine and the latter was also shown to hybridize with adenine and to enhance helical thermal stability by extended stacking interactions (also see U.S. Ser. No. 10/155,920 and U.S. Ser. No. 10/013,295, both of which are herein incorporated by reference in their entirety). Further helix-stabilizing properties have been observed when a cytosine analog/substitute has an aminoethoxy moiety attached to the rigid 1,3-diazaphenoxazine-2-one scaffold (Lin et al., Am. Chem. Soc., 120:8531-2, 1998).

### Internucleoside Linkage Modifications

Another variable in the design of the present disclosure is the internucleoside linkage making up the phosphate backbone of the siRNA molecule. Although the natural RNA phosphate backbone may be employed here, derivatives thereof may be used which enhance desirable characteristics of the siRNA molecule. Although not limiting, of particular importance in the present disclosure is protecting parts, or the whole, of the siRNA molecule from hydrolysis. One example of a modification that decreases the rate of hydrolysis is phosphorothioates. Any portion or the whole of the backbone may contain phosphate substitutions (e.g., phosphorothioates). For instance, the internucleoside linkages may be between 0 and 100% phosphorothioate, e.g., between 0 and 100%, 10 and 100%, 20 and 100%, 30 and 100%, 40 and 100%, 50 and 100%, 60 and 100%, 70 and 100%, 80 and 100%, 90 and 100%, 0 and 90%, 0 and 80%, 0 and 70%, 0 and 60%, 0 and 50%, 0 and 40%, 0 and 30%, 0 and 20%, 0 and 10%, 10 and 90%, 20 and 80%, 30 and 70%, 40 and 60%, 10 and 40%, 20 and 50%, 30 and 60%, 40 and 70%, 50 and 80%, or 60 and 90% phosphorothioate linkages. Similarly, the internucleoside linkages may be between 0 and 100% phosphodiester linkages, e.g., between 0 and 100%, 10 and 100%, 20 and 100%, 30 and 100%, 40 and 100%, 50 and 100%, 60 and 100% 70 and 100%, 80 and 100%, 90 and 100%, 0 and 90%, 0 and 80%, 0 and 70%, 0 and 60%, 0 and 50%, 0 and 40%, 0 and 30%, 0 and 20%, 0 and 10%, 10 and 90%, 20 and 80%, 30 and 70%, 40 and 60%, 10 and 40%, 20 and 50%, 30 and 60%, 40 and 70%, 50 and 80%, or 60 and 90% phosphodiester linkages.

Specific examples of some potential siRNA molecules useful in this invention include oligonucleotides containing modified e.g., non-naturally occurring internucleoside linkages. As defined in this specification, oligonucleotides having modified internucleoside linkages include internucleoside linkages that retain a phosphorus atom and internucleoside linkages that do not have a phosphorus atom. For the purposes of this specification, and as sometimes referenced in the art, modified oligonucleotides that do not have a phosphorus atom in their internucleoside backbone can also be considered to be oligonucleosides. A preferred phosphorus containing modified internucleoside linkage is the phosphorothioate internucleoside linkage. In some embodiments, the modified oligonucleotide backbones containing a phosphorus atom therein include, for example, phosphorothioates, phosphorodithioates, phosphotriesters, aminoalkylphosphotriesters, methyl and other alkyl phosphonates including 3′-alkylene phosphonates, 5′-alkylene phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates, and boranophosphates having normal 3′-5′ linkages, 2′-5′ linked analogs of these, and those having inverted polarity wherein one or more internucleoside linkages is a 3′ to 3′, 5′ to 5′ or 2′ to 2′ linkage. Exemplary U.S. patents describing the preparation of phosphorus-containing linkages include but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; 5,625,050; 6,028,188; 6,124,445; 6,160,109; 6,169,170; 6,172,209; 6,239,265; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; and U.S. Pat. No. RE39464, the entire contents of each of which are hereby incorporated herein by reference.

In some embodiments, the modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide, and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts. Non-limiting examples of U.S. patents that teach the preparation of non-phosphorus backbones include, but are not limited to, U.S. Pat. Nos. 5,034,506; 5,166,315; 5,185,444; 5,214,134; 5,216,141; 5,235,033; 5,64,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and 5,677,439, the entire contents of each of which are hereby incorporated herein by reference.

### Intersubunit Modifications

Alternatively, or in addition to the modifications discussed above, this section discloses additional intersubunit modifications that may be contained within the siRNA molecules of the disclosure. The siRNA molecules may contain at least 1 (e.g., at least 2, at least 3, at least 4, at least 5, at least 6, or more) intersubunit linkage modifications as disclosed herein. These modifications may be present within the fixed or overhang region of the antisense strand. In some embodiments, there may be 1, 2, 3, 4, 5, or more modifications as described in this section in a second region of the antisense strand that overhangs the sense strand.

In some embodiments, at least one modified intersubunit linkage is of Formula E1:

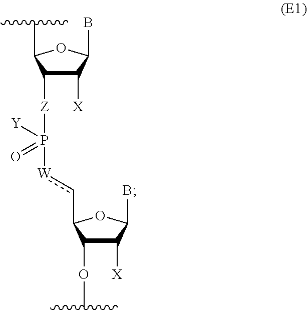

wherein:
   each B is, independently, a base pairing moiety;
   W is selected from the group consisting of O, S, B, BR², N, NR², OCH₂, OCH, CH₂, and CH, optionally wherein W is selected from the group consisting of OCH₂ and OCH;
   each X is, independently, selected from the group consisting of halo (e.g., fluoro or chloro), hydroxy, and C₁₋₆ alkoxy, optionally wherein each X is, independently, selected from the group consisting of halo (e.g., fluoro or chloro) and C₁₋₆ alkoxy (e.g., methoxy, ethoxy, 2-methoxyethoxy, n-propoxy, sec-propoxy, n-butoxy, sec-butoxy, tert-butoxy, n-pentoxy, or n-heptoxy);
   Y is selected from the group consisting of O⁻, OH, OR, NH⁻, NH₂, S⁻, and SH, optionally wherein Y is selected from the group consisting of O⁻, OH, and OR.
Z is selected from the group consisting of O, S, BR², NR², and CH₂;
R is a protecting group;
each R² is, independently, H or optionally substituted C₁-C₆ alkyl; and
[symbol] is an optional double bond.

In some embodiments of Formula E1, W is OCH₂.

In some embodiments of Formula E1, W is OCH and [symbol] is a double bond.

In some embodiments of Formula E1, Z is O.

In some embodiments of Formula E1, Z is CH₂.

In some embodiments of Formula E1, when Y is O⁻, either Z or W is not O.

In some embodiments of Formula E1, Z is CH₂ and W is CH₂. In some embodiments, the modified intersubunit linkage of Formula E1 is a modified intersubunit linkage of Formula E2:

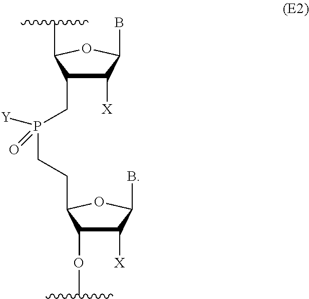

In some embodiments of Formula E1, Z is CH₂ and W is O. In some embodiments, the modified intersubunit linkage of Formula E1 is a modified intersubunit linkage of Formula E3:

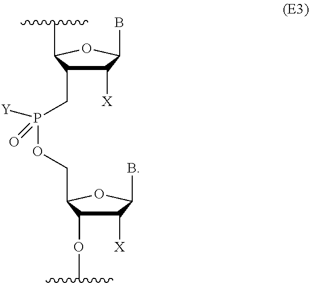

In some embodiments of Formula E1, Z is O and W is CH₂. In some embodiments, the modified intersubunit linkage of Formula E1 is a modified intersubunit linkage of Formula E4:

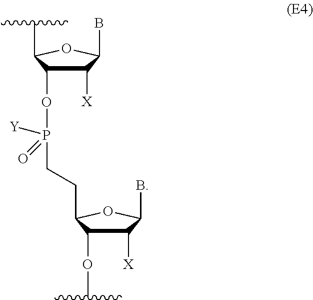

In some embodiments of Formula E1, Z is O and W is CH. In some embodiments, the modified intersubunit linkage of Formula E1 is a modified intersubunit linkage of Formula E5:

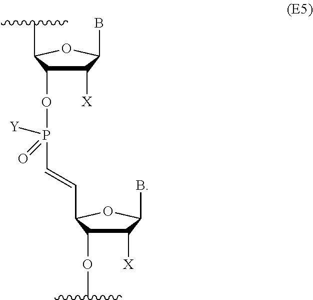

In some embodiments, the modified intersubunit linkage of Formula E1 is a modified intersubunit linkage of Formula E6:

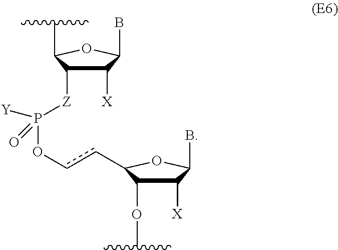

In some embodiments of Formula E6:
each B is, independently, a base pairing moiety;
   each X is, independently, selected from the group consisting of halo, hydroxy, and C₁₋₆ alkoxy; optionally wherein each X is, independently, selected from the group consisting of halo (e.g., fluoro) and C₁₋₆ alkoxy (e.g., methoxy, ethoxy, n-propoxy, sec-propoxy, n-butoxy, sec-butoxy, tert-butoxy, n-pentoxy, or n-heptoxy);
   Y is selected from the group consisting of O⁻, OH, OR, NH⁻, NH₂, S⁻, and SH, optionally wherein Y is selected from the group consisting of O⁻, OH, and OR;
Z is selected from the group consisting of O and CH₂; and
[symbol] is an optional double bond.

In some embodiments of Formula E6:
each X is, independently, selected from the group consisting of fluoro, hydroxy, and C₁₋₆ alkoxy; optionally wherein each X is, independently, selected from the group consisting of fluoro and C₁₋₆ alkoxy (e.g., methoxy, ethoxy, n-propoxy, sec-propoxy, n-butoxy, sec-butoxy, tert-butoxy, n-pentoxy, or n-heptoxy);
Y is selected from the group consisting of O⁻, OH, and OR;
Z is selected from the group consisting of O and CH₂; and
[symbol] is an optional double bond.

In some embodiments of Formula E6:
each X is, independently, selected from the group consisting of fluoro, hydroxy, methoxy, ethoxy, n-propoxy, sec-propoxy, n-butoxy, sec-butoxy, tert-butoxy, n-pentoxy, and n-heptoxy;
Y is selected from the group consisting of O⁻, OH, and OR;
Z is selected from the group consisting of O and CH₂; and
[symbol] is an optional double bond.

In some embodiments of Formula E6:
each X is, independently, selected from the group consisting of fluoro, hydroxy, methoxy, ethoxy, n-propoxy, sec-propoxy, n-butoxy, sec-butoxy, tert-butoxy, n-pentoxy, and n-heptoxy;
Y is selected from the group consisting of O⁻, OH, and OR;
Z is O; and
[symbol] is an optional double bond.

In some embodiments of Formula E6:
each X is, independently, selected from the group consisting of fluoro, hydroxy, methoxy, ethoxy, n-propoxy, sec-propoxy, n-butoxy, sec-butoxy, tert-butoxy, n-pentoxy, and n-heptoxy;
Y is selected from the group consisting of O⁻, OH, and OR;
Z is CH₂; and
[symbol] is an optional double bond.

In some embodiments of Formula E6:
each X is, independently, selected from the group consisting of fluoro, hydroxy, and methoxy;
Y is selected from the group consisting of O⁻, OH, and OR;
Z is O; and
[symbol] is an optional double bond.

In some embodiments of Formula E6:
each X is, independently, selected from the group consisting of fluoro, hydroxy, and methoxy;
Y is selected from the group consisting of O⁻, OH, and OR;
Z is CH₂; and
[symbol] is an optional double bond.

In some embodiments of Formula E1, Z is O and W is OCH₂. In some embodiments, the modified intersubunit linkage of Formula E1 is a modified intersubunit linkage of Formula E6a:

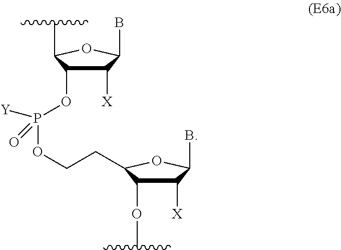

In some embodiments of Formula E1, Z is CH₂ and W is CH. In some embodiments, the modified intersubunit linkage of Formula E1 is a modified intersubunit linkage of Formula E7:

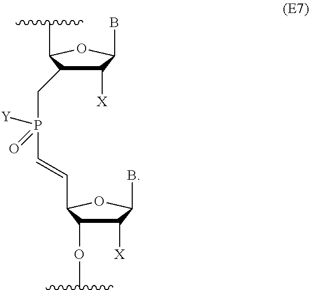

In some embodiments of Formula E1, the base pairing moiety B is selected from the group consisting of adenine, guanine, cytosine, and uracil.

In some embodiments, at least one modified intersubunit linkage is of Formula E8:

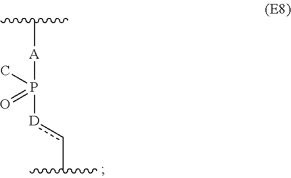

wherein:
   D is selected from the group consisting of O, S, B, BR², N, NR², OCH₂, OCH, CH₂, and CH, optionally wherein D is selected from the group consisting of OCH₂ and OCH;
   C is selected from the group consisting of O⁻, OH, OR¹, NH⁻, NH₂, S⁻, and SH, optionally wherein C is selected from the group consisting of O⁻, OH, and OR¹;
   A is selected from the group consisting of O, S, BR², NR², and CH₂;
   R1 is a protecting group;
   each R² is, independently, H or optionally substituted C₁-C₆ alkyl;
   [symbol] is an optional double bond; and
   the intersubunit is bridging two optionally modified nucleosides.

In some embodiments, D is OCH₂.

In some embodiments, D is OCH and [symbol] is a double bond.

In some embodiments, A is O.

In some embodiments, A is CH₂.

In some embodiments, when C is O⁻, either A or D is not O.

In some embodiments, D is CH₂. In another embodiment, the modified intersubunit linkage of Formula E8 is a modified intersubunit linkage of Formula F9.

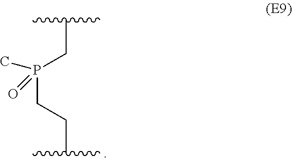 [SMILES: *CCP(C)(=O)C*]

In some embodiments, D is O. In another embodiment, the modified intersubunit linkage of Formula E8 is a modified intersubunit linkage of Formula E10:

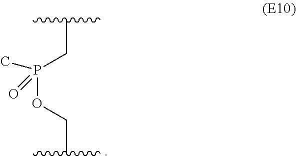 [SMILES: *COP(C)(=O)C*]

In some embodiments, D is CH₂. In another embodiment, the modified intersubunit linkage of Formula E8 is a modified intersubunit linkage of Formula E11:

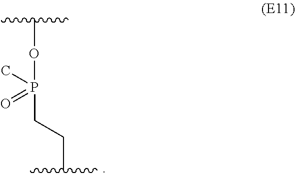 [SMILES: *CCP(C)(=O)O*]

In some embodiments, D is CH. In another embodiment, the modified intersubunit linkage of Formula E8 is a modified intersubunit linkage of Formula E12:

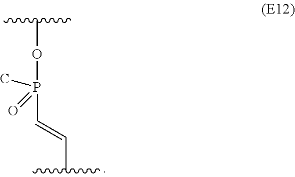 [SMILES: */C=C/P(C)(=O)O*]

In some embodiments, D is OCH₂. In another embodiment, the modified intersubunit linkage of Formula E8 is a modified intersubunit linkage of Formula E13:

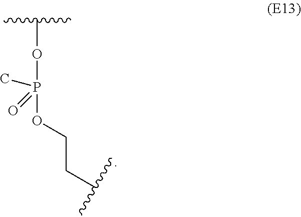 [SMILES: *CCOP(C)(=O)O*]

In another embodiment, the modified intersubunit linkage of Formula E8 is a modified intersubunit linkage of Formula E14:

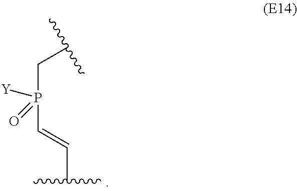

In another embodiment, the modified intersubunit linkage of Formula E8 is a modified intersubunit linkage of Formula E15:

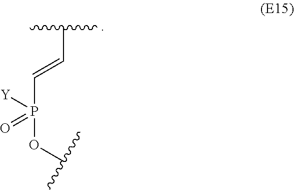

In some embodiments of the modified siRNA linkage, each optionally modified nucleoside is independently, at each occurrence, selected from the group consisting of adenosine, guanosine, cytidine, and uridine.

In some embodiments, at least one modified intersubunit linkage is of Formula E8:

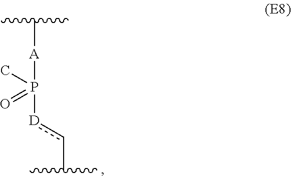

wherein:
   D is selected from the group consisting of O, OCH₂, OCH, CH₂, and CH, optionally wherein D is selected from the group consisting of OCH₂ and OCH;
   C is selected from the group consisting of O⁻, OH, OR¹, NH⁻, NH₂, S⁻, and SH, optionally wherein C is selected from the group consisting of O⁻, OH, and OR¹;
   A is selected from the group consisting of O and CH₂;
R¹ is a protecting group selected from the group consisting of dimethoxytrityl (DMTr), succinate, tert-butyl dimethylsilyl (TBDMS), benzoyl (Bz), benzyl (Bn), methoxyethoxymethyl ether (MOM), methoxybenzyl ether (PMB), methylthiomethyl ether, pivaloyl (Piv), tetrahydropyranyl (THP), tetrahydrofuranyl (THF), trityl (Trt), triisopropylsilyl (TIPS), tert-butyldiphenylsilyl (TBDPS), and acetate;
[symbol] is an optional double bond;
and the intersubunit is bridging two optionally modified nucleosides.

In some embodiments, D is OCH₂.

In some embodiments, D is OCH and [symbol] is a double bond.

In some embodiments, A is O.

In some embodiments, A is CH₂.

In some embodiments of any of the modified intersubunit linkages described above, the base pairing moiety B is selected from the group consisting of adenine, guanine, cytosine, and uracil.

In some embodiments, R is a protecting group selected from the group consisting of dimethoxytrityl (DMTr), succinate, tert-butyl dimethylsilyl (TBDMS), benzoyl (Bz), benzyl (Bn), methoxyethoxymethyl ether (MOM), methoxybenzyl ether (PMB), methylthiomethyl ether, pivaloyl (Piv), tetrahydropyranyl (THP), tetrahydrofuranyl (THF), trityl (Trt), triisopropylsilyl (TIPS), tert-butyldiphenylsilyl (TBDPS), and acetate; and
[symbol] is an optional double bond.
   Patterns of Modifications of siRNA Molecules

The following section provides a set of exemplary scaffolds into which the siRNA molecules of the disclosure may be incorporated.

In some embodiments of the disclosure, the siRNA may contain an antisense strand including a region represented by Formula I, wherein Formula I is, in the 5′-to-3′ direction:

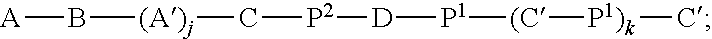

wherein A is represented by the formula C-P¹-D-P¹; each A′ is represented by the formula C-P²-D-P²; B is represented by the formula C-P²-D-P²-D-P²-D-P²; each C is a 2′-O-methyl(2′-O-Me) ribonucleoside; each C′, independently, is a 2′-O-Me ribonucleoside or a 2′-fluoro (2′-F) ribonucleoside; each D is a 2′-F ribonucleoside; each P¹ is a phosphorothioate internucleoside linkage; each P² is a phosphodiester internucleoside linkage; j is an integer from 1 to 7 (e.g., 1, 2, 3, 4, 5, 6, or 7); and k is an integer from 1 to 7 (e.g., 1, 2, 3, 4, 5, 6, or 7). In some embodiments, j is 4. In some embodiments, k is 4. In some embodiments, j is 4 and k is 4. The antisense is complementary (e.g., fully or partially complementary) to a target nucleic acid sequence.

In some embodiments, the antisense strand includes a structure represented by Formula A1, wherein Formula A1 is, in the 5′-to-3′ direction:

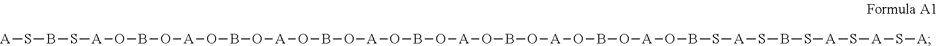

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the siRNA may contain an antisense strand including a region represented by Formula II, wherein Formula II is, in the 5′-to-3′ direction:

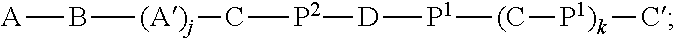

wherein A is represented by the formula C-P¹-D-P¹; each A′ is represented by the formula C-P²-D-P²; B is represented by the formula C-P²-D-P²-D-P²-D-P²; each C is a 2′-O-methyl(2′-O-Me) ribonucleoside; each C′, independently, is a 2′-O-Me ribonucleoside or a 2′-fluoro (2′-F) ribonucleoside; each D is a 2′-F ribonucleoside; each P¹ is a phosphorothioate internucleoside linkage; each P² is a phosphodiester internucleoside linkage; j is an integer from 1 to 7 (e.g., 1, 2, 3, 4, 5, 6, or 7); and k is an integer from 1 to 7 (e.g., 1, 2, 3, 4, 5, 6, or 7). In some embodiments, j is 4. In some embodiments, k is 4. In some embodiments, j is 4 and k is 4. The antisense is complementary (e.g., fully or partially complementary) to a target nucleic acid sequence.

In some embodiments of the disclosure, the antisense strand includes a structure represented by Formula A2, wherein Formula A2 is, in the 5′-to-3′ direction:

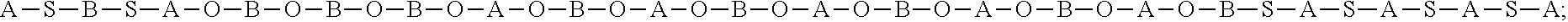

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula III, wherein Formula III is, in the 5′-to-3′ direction:

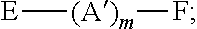

wherein E is represented by the formula (C-P¹)₂; F is represented by the formula (C-P²)₃-D-P¹-C-P¹-C, (C-P²)₃-D-P²-C-P²-C, (C-P²)₃-D-P¹-C-P¹-D, or (C-P²)₃-D-P²-C-P²-D; A′, C, D, P¹, and P² are as defined in Formula I; and m is an integer from 1 to 7 (e.g., 1, 2, 3, 4, 5, 6, or 7). In some embodiments, m is 4. The sense strand is complementary (e.g., fully or partially complementary) to the antisense strand.

In some embodiments of the disclosure, the sense strand includes a structure represented by Formula S1, wherein Formula S1 is, in the 5′-to-3′ direction:

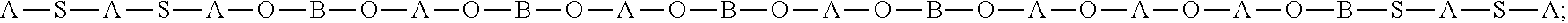

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula S2, wherein Formula S2 is, in the 5′-to-3′ direction:

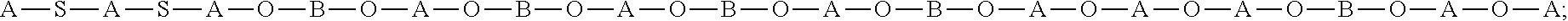

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula S3, wherein Formula S3 is, in the 5′-to-3′ direction:

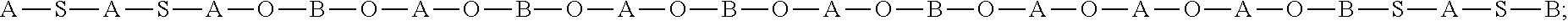

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula S4, wherein Formula S4 is, in the 5′-to-3′ direction:

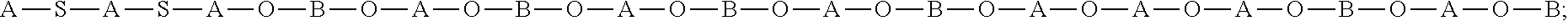

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the siRNA may contain an antisense strand including a region represented by Formula IV, wherein Formula IV is, in the 5′-to-3′ direction:

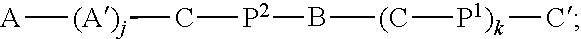

wherein A is represented by the formula C-P¹-D-P¹; each A′ is represented by the formula C-P²-D-P²; B is represented by the formula D-P¹-C-P¹-D-P¹; each C is a 2′-O-Me ribonucleoside; each C′, independently, is a 2′-O-Me ribonucleoside or a 2′-F ribonucleoside; each D is a 2′-F ribonucleoside; each P¹ is a phosphorothioate internucleoside linkage; each P² is a phosphodiester internucleoside linkage; j is an integer from 1 to 7 (e.g., 1, 2, 3, 4, 5, 6, or 7); and k is an integer from 1 to 7 (e.g., 1, 2, 3, 4, 5, 6, or 7). In some embodiments, j is 6. In some embodiments, k is 4. In some embodiments, j is 6 and k is 4. The antisense strand is complementary (e.g., fully or partially complementary) to a target nucleic acid.

In some embodiments of the disclosure, the antisense strand includes a structure represented by Formula A3, wherein Formula A3 is, in the 5′-to-3′ direction:

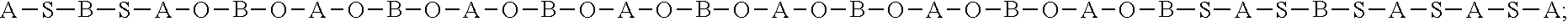

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the siRNA of the disclosure may have a sense strand represented by Formula V, wherein Formula V is, in the 5′-to-3′ direction:

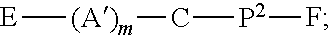

wherein E is represented by the formula (C-P¹)₂; F is represented by the formula D-P¹-C-P¹-C, D-P²-C-P²-C, D-P¹-C-P¹-D, or D-P²-C-P²-D; A′, C, D, P¹, and P² are as defined in Formula IV; and m is an integer from 1 to 7 (e.g., 1, 2, 3, 4, 5, 6, or 7). In some embodiments, m is 5. The sense strand is complementary (e.g., fully or partially complementary) to the antisense strand.

In some embodiments of the disclosure, the sense strand includes a structure represented by Formula S5, wherein Formula S5 is, in the 5′-to-3′ direction:

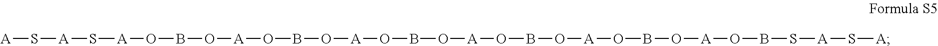

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula S6, wherein Formula S6 is, in the 5′-to-3′ direction:

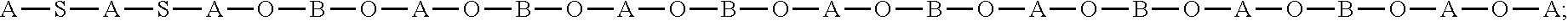

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula S7, wherein Formula S7 is, in the 5′-to-3′ direction:

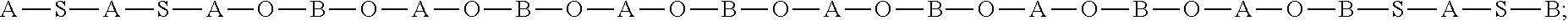

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula S8, wherein Formula S8 is, in the 5′-to-3′ direction:

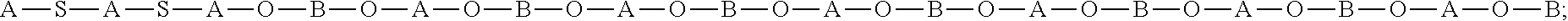

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the siRNA may contain an antisense strand including a region represented by Formula VI, wherein Formula VI is, in the 5′-to-3′ direction:

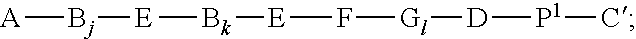

wherein A is represented by the formula C-P¹-D-P¹; each B is represented by the formula C-P²; each C is a 2′-O-Me ribonucleoside; each C′, independently, is a 2′-O-Me ribonucleoside or a 2′-F ribonucleoside; each D is a 2′-F ribonucleoside; each E is represented by the formula D-P²-C-P²; F is represented by the formula D-P¹-C-P¹; each G is represented by the formula C-P¹; each P¹ is a phosphorothioate internucleoside linkage; each P² is a phosphodiester internucleoside linkage; j is an integer from 1 to 7 (e.g., 1, 2, 3, 4, 5, 6, or 7); k is an integer from 1 to 7 (e.g., 1, 2, 3, 4, 5, 6, or 7); and l is an integer from 1 to 7 (e.g., 1, 2, 3, 4, 5, 6, or 7). In some embodiments, j is 3. In some embodiments, k is 6. In some embodiments, l is 2. In some embodiments, j is 3, k is 6, and l is 2. The antisense strand is complementary (e.g., fully or partially complementary) to a target nucleic acid.

In some embodiments of the disclosure, the antisense strand includes a structure represented by Formula A4, wherein Formula A4 is, in the 5′-to-3′ direction:

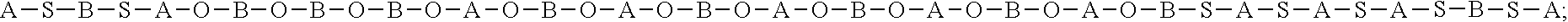

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the siRNA may contain a sense strand including a region represented by Formula VII, wherein Formula VII is, in the 5′-to-3′ direction:

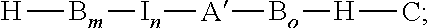

wherein A′ is represented by the formula C-P²-D-P²; each H is represented by the formula (C-P¹)₂; each I is represented by the formula (D-P²); B, C, D, P¹, and P² are as defined in Formula VI; m is an integer from 1 to 7 (e.g., 1, 2, 3, 4, 5, 6, or 7); n is an integer from 1 to 7 (e.g., 1, 2, 3, 4, 5, 6, or 7); and o is an integer from 1 to 7 (e.g., 1, 2, 3, 4, 5, 6, or 7). In some embodiments, m is 3. In some embodiments, n is 3. In some embodiments, o is 3. In some embodiments, m is 3, n is 3, and o is 3. The sense strand is complementary (e.g., fully or partially complementary) to the antisense strand.

In some embodiments of the disclosure, the sense strand includes a structure represented by Formula S9, wherein Formula S9 is, in the 5′-to-3′ direction:

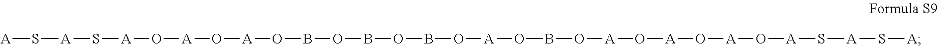

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the siRNA may contain an antisense strand including a region that is represented by Formula VIII:

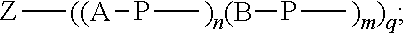

wherein Z is a 5′ phosphorus stabilizing moiety; each A is a 2′-O-methyl(2′-O-Me) ribonucleoside; each B is a 2′-fluoro-ribonucleoside; each P is, independently, an internucleoside linkage selected from a phosphodiester linkage and a phosphorothioate linkage; n is an integer from 1 to 5 (e.g., 1, 2, 3, 4, or 5); m is an integer from 1 to 5 (e.g., 1, 2, 3, 4, or 5); and q is an integer between 1 and 30 (1, 2, 3, 4, 5, 6, 7, 8, 9, 10, 11, 12, 13, 14, 15, 16, 17, 18, 19, 20, 21, 22, 23, 24, 25, 26, 27, 28, 29, or 30).
   Methods of siRNA Synthesis

The siRNA molecules of the disclosure can be synthesized by standard methods known in the art as further discussed below, e.g., by use of an automated DNA synthesizer, such as are commercially available from, for example, Biosearch, Applied Biosystems, Inc.

The siRNA agent can be prepared using solution-phase or solid-phase organic synthesis or both. Organic synthesis offers the advantage that the oligonucleotide including unnatural or modified nucleotides can be easily prepared. siRNA molecules of the disclosure can be prepared using solution-phase or solid-phase organic synthesis or both.

Further, it is contemplated that for any siRNA agent disclosed herein, further optimization could be achieved by systematically either adding or removing linked nucleosides to generate longer or shorter sequences. Further still, such optimized sequences can be adjusted by, e.g., the introduction of modified nucleosides, and/or modified internucleoside linkages as described herein or as known in the art, including alternative nucleosides, alternative sugar moieties, and/or alternative internucleoside linkages as known in the art and/or discussed herein to further optimize the molecule (e.g., increasing serum stability or circulating half-life, increasing thermal stability, enhancing transmembrane delivery, and/or targeting to a particular location or cell type).

### 5′ Phosphorus Stabilizing Moieties

To further protect the siRNA molecules of this disclosure from degradation, a 5′-phosphorus stabilizing moiety may be employed. A 5′-phosphorus stabilizing moiety replaces the 5′-phosphate to prevent hydrolysis of the phosphate. Hydrolysis of the 5′-phosphate prevents binding to RISC, a necessary step in gene silencing. Any replacement for phosphate that does not impede binding to RISC is contemplated in this disclosure. In some embodiments, the replacement for the 5′-phosphate is also stable to in vivo hydrolysis. Each strand of a siRNA molecule may independently and optionally employ any suitable 5′-phosphorus stabilizing moiety.

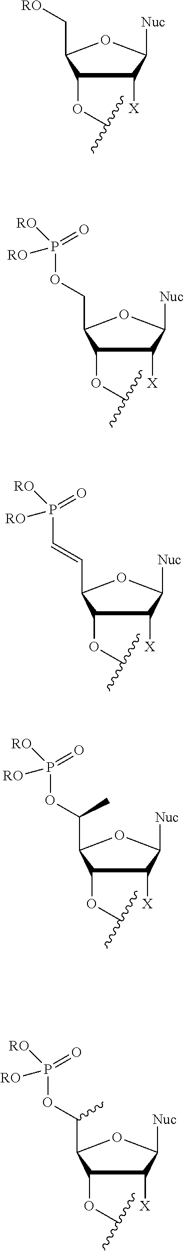

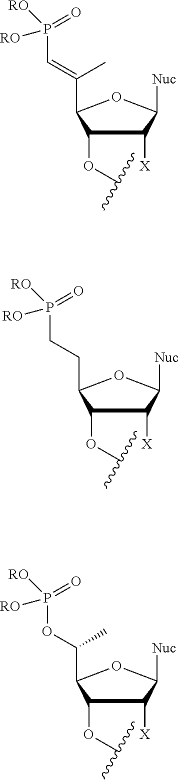

Some exemplary endcaps are demonstrated in Formulas IX-XVI. Nuc in Formulas IX-XVI represents a nucleobase or nucleobase derivative or replacement as described herein. X in formula IX-XVI represents a 2′-modification as described herein. Some embodiments employ hydroxy as in Formula IX, phosphate as in Formula X, vinylphosphonates as in Formula XI and XIV, 5′-methyl-substituted phosphates as in Formula XII, XIII, and XVI, methylenephosphonates as in Formula XV, or vinyl 5′-vinylphsophonate as a 5′-phosphorus stabilizing moiety as demonstrated in Formula XI.

### Hydrophobic Moieties

The present disclosure further provides siRNA molecules having one or more hydrophobic moieties attached thereto. The hydrophobic moiety may be covalently attached to the 5′ end or the 3′ end of the siRNA molecules of the disclosure. Non-limiting examples of hydrophobic moieties suitable for use with the siRNA molecules of the disclosure may include cholesterol, vitamin D, tocopherol, phosphatidylcholine (PC), docosahexaenoic acid, docosanoic acid, PC-docosanoic acid, eicosapentaenoic acid, lithocholic acid or any combination of the aforementioned hydrophobic moieties with PC.

siRNA Branching

The siRNA molecules of the disclosure may be branched. For example, the siRNA molecules of the disclosure may have one of several branching patterns, as described herein.

According to the present disclosure, the siRNA molecules disclosed herein may be branched siRNA molecules. The siRNA molecule may not be branched, or may be di-branched, tri-branched, or tetra-branched, connected through a linker. Each main branch may be further branched to allow for 2, 3, 4, 5, 6, 7, or 8 separate RNA single- or double-strands. The branch points on the linker may stem from the same atom, or separate atoms along the linker. Some exemplary embodiments are listed in Table 2.

TABLE 2
Branched siRNA Structures

| Di-branched | Tri-branched | Tetra-branched |
|---|---|---|
| RN-L-RNA Formula XVII | 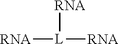 | 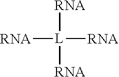 |
| | Formula XX | Formula XXIV |
| 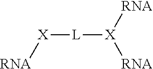 | 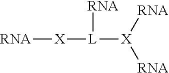 | 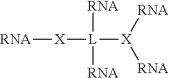 |
| Formula XVIII | Formula XXI | Formula XXV |
| 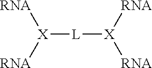 | 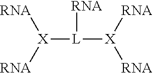 | 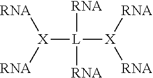 |
| Formula XIX | Formula XXII | Formula XXVI |
| | 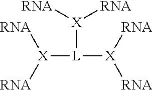 | 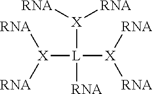 |
| | Formula XXIII | Formula XXVII |
| | | 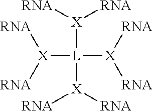 |
| | | Formula XXVIII |

In some embodiments, the siRNA molecule is a branched siRNA molecule. In some embodiments, the branched siRNA molecule is di-branched, tri-branched, or tetra-branched. In some embodiments, the di-branched siRNA molecule is represented by any one of Formulas XVII-XIX, wherein each RNA, independently, is an siRNA molecule, L is a linker, and each X, independently, represents a branch point moiety (e.g., phosphoroamidite, tosylated solketal, 1,3-diaminopropanol, pentaerythritol, or any one of the branch point moieties described in U.S. Pat. No. 10,478,503).

In some embodiments, the tri-branched siRNA molecule represented by any one of Formulas XX-XXIII, wherein each RNA, independently, is an siRNA molecule, L is a linker, and each X, independently, represents a branch point moiety.

In some embodiments, the tetra-branched siRNA molecule represented by any one of Formulas XXIV-XXVIII, wherein each RNA, independently, is an siRNA molecule, L is a linker, and each X, independently, represents a branch point moiety.

### Linkers

Multiple strands of siRNA described herein may be covalently attached by way of a linker. The effect of this branching improves, inter alia, cell permeability allowing better access into cells (e.g., neurons or glial cells) in the CNS. Any linking moiety may be employed which is not incompatible with the siRNAs of the present invention. Linkers include ethylene glycol chains of 2 to 10 subunits (e.g., 2, 3, 4, 5, 6, 7, 8, 9, or 10 subunits), alkyl chains, carbohydrate chains, block copolymers, peptides, RNA, DNA, and others. In some embodiments, any carbon or oxygen atom of the linker is optionally replaced with a nitrogen atom, bears a hydroxyl substituent, or bears an oxo substituent. In some embodiments, the linker is a poly-ethylene glycol (PEG) linker. The PEG linkers suitable for use with the disclosed compositions and methods include linear or non-linear PEG linkers. Examples of non-linear PEG linkers include branched PEGs, linear forked PEGs, or branched forked PEGs.

PEG linkers of various weights may be used with the disclosed compositions and methods. For example, the PEG linker may have a weight that is between 5 and 500 Daltons. In some embodiments, a PEG linker having a weight that is between 500 and 1,000 Dalton may be used. In some embodiments, a PEG linker having a weight that is between 1,000 and 10,000 Dalton may be used. In some embodiments, a PEG linker having a weight that is between 200 and 20,000 Dalton may be used. In some embodiments, the linker is covalently attached to a sense strand of the siRNA. In some embodiments, the linker is covalently attached to an antisense strand of the siRNA. In some embodiments, the PEG linker is a triethylene glycol (TrEG) linker. In some embodiments, the PEG linker is a tetraethylene glycol (TEG) linker.

In some embodiments, the linker is an alkyl chain linker. In some embodiments, the linker is a peptide linker. In some embodiments, the linker is an RNA linker. In some embodiments, the linker is a DNA linker.

Linkers may covalently link 2, 3, 4, or 5 unique siRNA strands. The linker may covalently bind to any part of the siRNA oligomer. In some embodiments, the linker attaches to the 3′ end of nucleosides of each siRNA strand. In some embodiments, the linker attaches to the 5′ end of nucleosides of each siRNA strand. In some embodiments, the linker attaches to a nucleoside of an siRNA strand (e.g., sense or antisense strand) by way of a covalent bond-forming moiety. In some embodiments, the covalent-bond-forming moiety is selected from the group consisting of an alkyl, ester, amide, carbonate, carbamate, triazole, urea, formacetal, phosphonate, phosphate, and phosphate derivative (e.g., phosphorothioate, phosphoramidate, etc.).

In some embodiments, the linker has a structure of Formula L1:

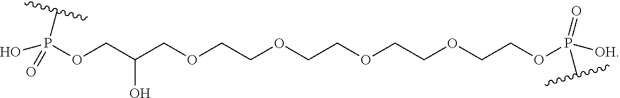 [SMILES: *P(=O)(O)OCCOCCOCCOCCOCC(O)COP(*)(=O)O]

In some embodiments, the linker has a structure of Formula L2:

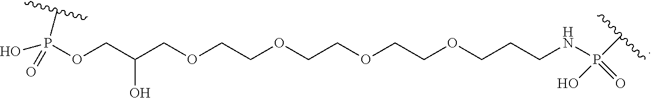 [SMILES: *P(=O)(O)NCCCOCCOCCOCCOCC(O)COP(*)(=O)O]

In some embodiments, the linker has a structure of Formula L3:

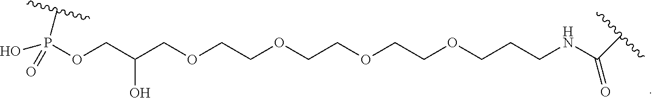 [SMILES: *C(=O)NCCCOCCOCCOCCOCC(O)COP(*)(=O)O]

In some embodiments, the linker has a structure of Formula L4:

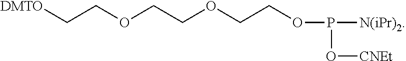

In some embodiments, the linker has a structure of Formula L5:

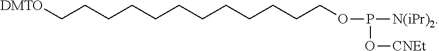

In some embodiments, the linker has a structure of Formula L6:

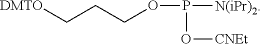

In some embodiments, the linker has a structure of Formula L7, as is shown below:

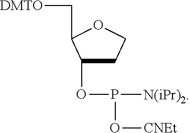

In some embodiments, the linker has a structure of Formula L8:

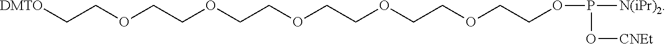

In some embodiments, the linker has a structure of Formula L9:

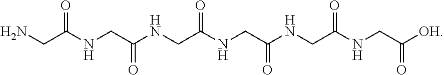 [SMILES: NCC(=O)NCC(=O)NCC(=O)NCC(=O)NCC(=O)NCC(=O)O]

In some embodiments, the selection of a linker for use with one or more of the branched siRNA molecules disclosed herein may be based on the hydrophobicity of the linker, such that, e.g., desirable hydrophobicity is achieved for the one or more branched siRNA molecules of the disclosure. For example, a linker containing an alkyl chain may be used to increase the hydrophobicity of the branched siRNA molecule as compared to a branched siRNA molecule having a less hydrophobic linker or a hydrophilic linker.

The siRNA agents disclosed herein may be synthesized and/or modified by methods well established in the art, such as those described in Beaucage, S. L. et al. (edrs.), Current Protocols in Nucleic Acid Chemistry, John Wiley & Sons, Inc., New York, N.Y., 2000, which is hereby incorporated herein by reference.

### Methods of Treatment

The disclosure provides methods of treating a subject in need of gene silencing. The gene silencing may be performed in order to silence defective or overactive genes, silence negative regulators of genes with reduced expression, silence wild type genes with an activating role in a pathway(s) that increases activity of a disease driver gene, silence splice isoforms of a gene(s) that, when selectively knocked down, may elevate total expression of the gene(s), among other reasons, so long as the goal is to restore genetic and biochemical pathway activity from a disease state towards a healthy state. The method may include delivering to the CNS of the subject (e.g., a human) an siRNA molecule of the disclosure or a pharmaceutical composition containing the same by any appropriate route of administration (e.g., intrastriatal, intracerebroventricular, intrathecal injection, intrastriatal injection, intra-cisterna magna injection by catheterization, intraparenchymal injection, intravenous injection, subcutaneous injection, or intramuscular injection). The active compound can be administered in any suitable dose. The actual dosage amount of a composition of the present disclosure administered to a patient can be determined by physical and physiological factors such as body weight, severity of condition, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject. Administration may occur any suitable number of times per day, and for as long as necessary. Subjects may be adult or pediatric humans, with or without comorbid diseases.

### Selection of Subjects

Subjects that may be treated with the siRNA molecules disclosed herein are subjects in need of treatment of, for example, any medical risk(s) associated with a gain of function mutation in the target gene. Subjects that may be treated with the siRNA molecules disclosed herein may include, for example, humans, monkeys, rats, mice, pigs, and other mammals containing at least one orthologous copy of the target gene. Subjects may be adult or pediatric humans, with or without comorbid diseases.

### Pharmaceutical Compositions

The siRNA molecules in the present disclosure may be formulated into a pharmaceutical composition for administration to a subject in a biologically compatible form suitable for administration in vivo. Accordingly, the present disclosure provides a pharmaceutical composition containing an siRNA molecule of the disclosure in admixture with a suitable diluent, carrier, or excipient. The siRNA molecules may be administered, for example, directly into the CNS or affected tissues of the subject (e.g., by way of intracerebroventricular, intrastriatally, intrathecal injection, intra-cisterna magna injection by catheterization, intraparenchymal injection, intravenous injection, subcutaneous injection, or intramuscular injection).

Conventional procedures and ingredients for the selection and preparation of suitable formulations are described, for example, in Remington, J. P. The Science and Practice of Pharmacy, Easton, PA. Mack Publishers, 2012, 22^{nd} ed. and in The United States Pharmacopeial Convention, The National Formulary, United States Pharmacopeial, 2015, USP 38 NF 33).

Under ordinary conditions of storage and use, a pharmaceutical composition may contain a preservative, e.g., to prevent the growth of microorganisms. Pharmaceutical compositions may include sterile aqueous solutions, dispersions, or powders, e.g., for the extemporaneous preparation of sterile solutions or dispersions. In all cases the form may be sterilized using techniques known in the art and may be fluidized to the extent that may be easily administered to a subject in need of treatment.

A pharmaceutical composition may be administered to a subject, e.g., a human subject, alone or in combination with pharmaceutically acceptable carriers, as noted herein, the proportion of which may be determined by the solubility and/or chemical nature of the compound, chosen route of administration, and standard pharmaceutical practice.

### Dosing Regimens

A physician having ordinary skill in the art can readily determine an effective amount of the siRNA molecule for administration to a mammalian subject (e.g., a human) in need thereof. For example, a physician could start prescribing doses of one the siRNA molecules of the disclosure at levels lower than that required to achieve the desired therapeutic effect and gradually increase the dosage until the desired effect is achieved. Alternatively, a physician may begin a treatment regimen by administering one of the siRNA molecules of the disclosure at a high dose and subsequently administer progressively lower doses until reaching a minimal dosage at which a therapeutic effect is achieved (e.g., a reduction in expression of a target gene sequence). In general, a suitable daily dose of one of the siRNA molecules of the disclosure will be an amount of the siRNA molecule which is the lowest dose effective to produce a therapeutic effect. The ss- or ds-siRNA molecules of the disclosure may be administered by injection, e.g., intrathecally, intracerebroventricularly, by intra-cisterna magna injection by catheterization, intraparenchymally, intravenously, subcutaneously, or intramuscularly. A daily dose of a therapeutic composition of the siRNA molecules of the disclosure may be administered as a single dose or as two, three, four, five, six or more doses administered separately at appropriate intervals throughout the day, week, month, or year, optionally, in unit dosage forms. While it is possible for the siRNA molecules of the disclosure to be administered alone, it may also be administered as a pharmaceutical formulation in combination with excipients, carriers, and optionally, additional therapeutic agents.

### Routes of Administration

The method of the disclosure contemplates any route of administration tolerated by the therapeutic composition. Some embodiments of the method include injection intrathecally, intracerebroventricularly, intrastriatally, intraparenchymally, or by intra-cisterna magna injection by catheterization.

Intrathecal injection is the direct injection into the spinal column or subarachnoid space. By injecting directly into the CSF of the spinal column the siRNA molecules of the disclosure have direct access to cells (e.g., neurons and glial cells) in the spinal column and a route to access the cells in the brain by bypassing the blood brain barrier.

Intracerebroventricular (ICV) injection is a method to directly inject into the CSF of the cerebral ventricles. Similar to intrathecal injection, ICV is a method of injection which bypasses the blood brain barrier. Using ICV allows the advantage of access to the cells of the brain and spinal column without the danger of the therapeutic being degraded in the blood.

Intrastriatal injection is the direct injection into the striatum, or corpus striatum. The striatum is an area in the subcortical basal ganglia in the brain. Injecting into the striatum bypasses the blood brain barrier and the pharmacokinetic challenges of injection into the blood stream and allows for direct access to the cells of the brain.

Intraparenchymal administration is the direct injection into the parenchyma (e.g., the brain parenchyma). Injection into the brain parenchyma allows for injection directly into brain regions affected by a disease or disorder while bypassing the blood brain barrier.

Intra-cisterna magna injection by catheterization is the direct injection into the cisterna magna. The cisterna magna is the area of the brain located between the cerebellum and the dorsal surface of the medulla oblongata. Injecting into the cisterna magna results in more direct delivery to the cells of the cerebellum, brainstem, and spinal cord.

In some embodiments of the methods described herein, the therapeutic composition may be delivered to the subject by way of systemic administration, e.g., intravenously, intramuscularly, or subcutaneously.

Intravenous (IV) injection is a method to directly inject into the bloodstream of a subject. The IV administration may be in the form of a bolus dose or by way of continuous infusion, or any other method tolerated by the therapeutic composition.

Intramuscular (IM) injection is injection into a muscle of a subject, such as the deltoid muscle or gluteal muscle. IM may allow for rapid absorption of the therapeutic composition.

Subcutaneous injection is injection into subcutaneous tissue. Absorption of compositions delivered subcutaneously may be slower than IV or IM injection, which may be beneficial for compositions requiring continuous absorption.

### EXAMPLES

The following examples are put forth so as to provide those of ordinary skill in the art with a description of how the compositions and methods described herein may be used, made, and evaluated, and are intended to be purely exemplary of the disclosure and are not intended to limit the scope of what the inventors regard as their disclosure.

### Example 1. Effect of Fixed Nucleobase Sequences at the 3′End of an Antisense Strand of a Ds-siRNA Molecule

This example demonstrates the ability of PRNP-targeting siRNA molecules of the disclosure with a fixed nucleobase sequence at the 3′ end of the antisense strand. siRNA molecules with two uridines at the 3′ end of the antisense strand were compared with the sequence matched.

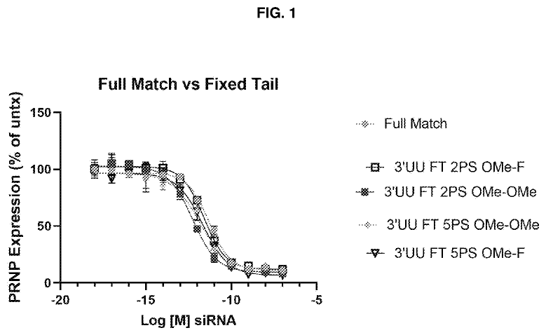

siRNA molecules targeting PRNP were delivered into Hela cells by lipid-mediated cellular uptake (RNAiMax). Hela cells were seeded and simultaneously transfected with varied concentrations of the siRNA molecules using RNAiMax. PRNP mRNA expression was measured 24 hours post transfection. FIG. 1 and Table 3 demonstrate that each of the siRNA molecules effectively silence PRNP.

TABLE 3

| siRNA | | | |
|---|---|---|---|
| ID | Description | 3′-Modification Pattern | IC50 |
| 1 | Sequence match, 5PS | -S-(mG)-S-(mC)-S-(mA)-S-(mA)-S-(mG) | 4.844 pM |
| 2 | 3′-UU (FT), 5PS, OMe—OMe | -S-(mG)-S-(mC)-S-(mA)-S-(mU)-S-(mU) | 1.340 pM |
| 3 | 3′-UU FT, 2PS, OMe—OMe | -O-(mG)-O-(mC)-O-(mA)-S-(mU)-S-(mU) | 0.5395 pM |
| 4 | 3′UU FT, 5PS, OMe—F | -S-(mG)-S-(mC)-S-(mA)-S-(mU)-S-(fU) | 2.096 pM |
| 5 | 3′UU FT, 2PS, OMe—F | -O-(mG)-O-(mC)-O-(mA)-S-(mU)-S-(fU) | 2.527 pM |

In Table 3, O represents a phosphodiester internucleoside linkage, S represents a phosphorothioate internucleoside linkage, mG represents a 2′-methoxy guanosine, mC is a 2′-methoxy cytosine, mA is a 2′-methoxy adenosine, mU is a 2′-methoxy uridine, and fU is a 2′-fluoro uridine.

### Example 2. Effect of Including Modified Internucleoside Linkages or Modified Ribose Moieties in Fixed Nucleobase Sequences at the 3′End of an Antisense Strand of a ds-siRNA Molecule

This example demonstrates the advantages of including modified internucleoside linkages in a fixed nucleobase region at the 3′ end of an antisense strand of an siRNA molecule. The modified internucleoside linkages include modified internucleoside linkages of any one of Formulas E1-E15 (e.g., a modified internucleoside linkage of Formula E6a) or phosphorothioate internucleoside linkages. Additionally, this example investigates the effect of including modified ribose moieties (e.g., 2′-methoxy nucleosides and/or 2′-fluoro nucleosides).

7- to 8-week-old PRNP Tg26378 mice were administered PBS control or siRNA molecules targeting a PRNP mRNA transcript by way of bilateral intracerebroventricular injection. 10 μL per animal (5 μL/vent) was injected at a rate of 2.0 μL/minute. Tissue was collected from the frontal cortex (fCtx), motor cortex (mCTx), striatum (Cpu), and hippocampus (HP). N=8 for each condition tested. The tissues were analyzed for the amount of PRNP mRNA expression relative to the PBS control. Each condition was separately tested with an injection of 0.2 nmol, 1 nmol, and 5 nmol of siRNA molecule. The siRNA molecules tested are described in Table 4, below.

TABLE 4

| | | 3′ |
|---|---|---|
| | | Chemical |
| | | Modifi- |
| siRNA | | cation |
| ID | Nucleobase Sequence | Pattern |
| 6 | Two terminal nucleobases at the 3′ end of the | Formula F1 |
| | antisense strand are fixed as UU; otherwise | |
| | sequence matched to a target PRNP transcript | |
| 7 | Two terminal nucleobases at the 3′ end of the | Formula F3 |
| | antisense strand are fixed as UU; otherwise | |
| | sequence matched to a target PRNP transcript | |
| 8 | Two terminal nucleobases at the 3′ end of the | Formula F5 |
| | antisense strand are fixed as UU; otherwise | |
| | sequence matched to a target PRNP transcript | |
| 9 | Two terminal nucleobases at the 3′ end of the | Formula F4 |
| | antisense strand are fixed as UU; otherwise | |
| | sequence matched to a target PRNP transcript | |
| 10 | Two terminal nucleobases at the 3′ end of the | Formula F6 |
| | antisense strand are fixed as UU; otherwise | |
| | sequence matched to a target PRNP transcript | |

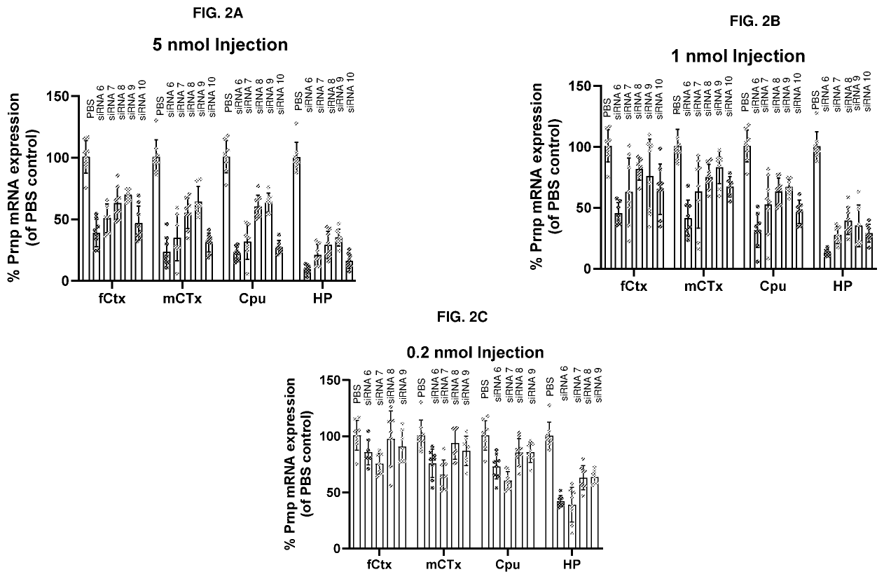

The results of this experiment are shown in FIGS. 2A (5 nmol injection) 2B (1 nmol injection) and 2C (0.2 nmol injection) with the results reported as the % of PRNP mRNA measured relative to control mice treated with PBS. These results demonstrate that including fixed nucleobase sequences effectively silence PRNP mRNA in vivo.

### Example 3. siRNA Molecules with Fixed Nucleobase Regions

This example further demonstrates the advantages of including modified internucleoside linkages in a fixed nucleobase region at the 3′ end of an antisense strand of an siRNA molecule. The modified internucleoside linkages include modified internucleoside linkages of any one of Formulas E1-E15 (e.g., a modified internucleoside linkage of Formula E6a) or phosphorothioate internucleoside linkages. Additionally, this example investigates the effect of including modified ribose moieties (e.g., 2′-methoxy nucleosides and/or 2′-fluoro nucleosides).

8- to 9-week-old FVB/N female mice were administered PBS control or siRNA molecules targeting a HPRT1 mRNA transcript by way of bilateral intracerebroventricular injection as described in Example 2. Tissue was collected from the frontal cortex (fCtx), motor cortex (mCTx), striatum (Cpu), and hippocampus (HP). N=8-10 for each condition tested. The tissues were analyzed after 28 days for HPRT1 mRNA expression relative to the PBS control. Each condition was separately tested with an injection of 1 nmol, or 3 nmol of siRNA molecule. The siRNA molecules tested are described in Table 5, below.

TABLE 5

| | | | 3′ |
|---|---|---|---|
| | | | Chemical |
| | | | Modifi- |
| siRNA | | | cation |
| ID | Nucleobase Sequence | Description | Pattern |
| 11 | Two terminal nucleobases at the | 3′-UU FT, 5PS, | Formula F1 |
| | 3′ end of the antisense strand are | OMe—OMe | |
| | fixed as UU; otherwise sequence | | |
| | matched to a target HPRT1 | | |
| | transcript | | |
| 12 | Two terminal nucleobases at the | 3′-UU FT, 5PS, | Formula F6 |
| | 3′ end of the antisense strand are | OMe—F | |
| | fixed as UU; otherwise sequence | | |
| | matched to a target HPRT1 | | |
| | transcript | | |
| 13 | Two terminal nucleobases at the | 3′-Ex(UU) FT, | Formula F3 |
| | 3′ end of the antisense strand are | 2PS, OMe—F | |
| | fixed as UU; otherwise sequence | | |
| | matched to a target HPRT1 | | |
| | transcript | | |
| 14 | Two terminal nucleobases at the | 3′-Ex(UU) FT, | Formula F4 |
| | 3′ end of the antisense strand are | 5PS, OMe—F | |
| | fixed as UU; otherwise sequence | | |
| | matched to a target HPRT1 | | |
| | transcript | | |
| 15 | Two terminal nucleobases at the | 3′-Ex(UU) FT, | Formula F5 |
| | 3′ end of the antisense strand are | 5PS, | |
| | fixed as UU; otherwise sequence | OMe—OMe | |
| | matched to a target HPRT1 | | |
| | transcript | | |
| 16 | Fully sequence matched to a | Seq. Matched, | Formula F1 |
| | target HPRT1 transcript | 5PS, | |
| | | OMe—OMe | |

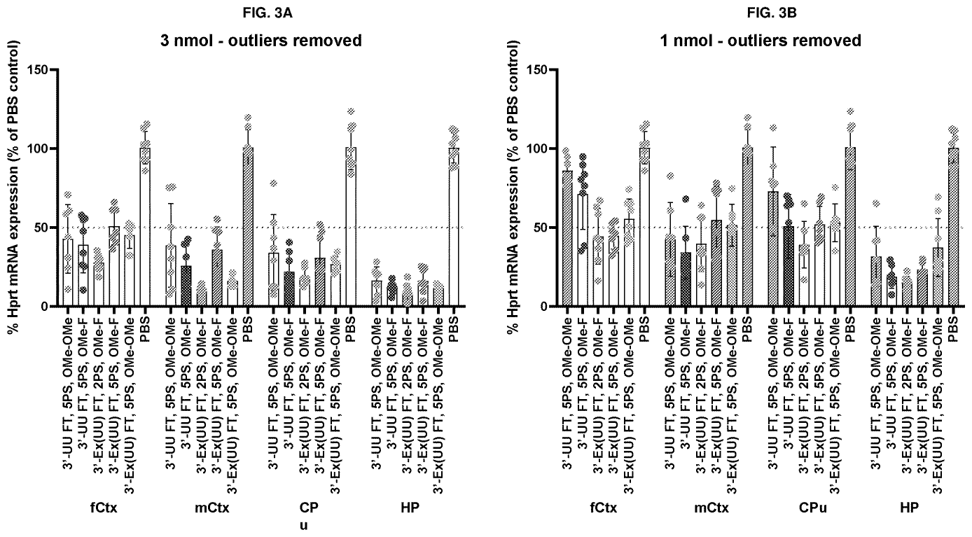

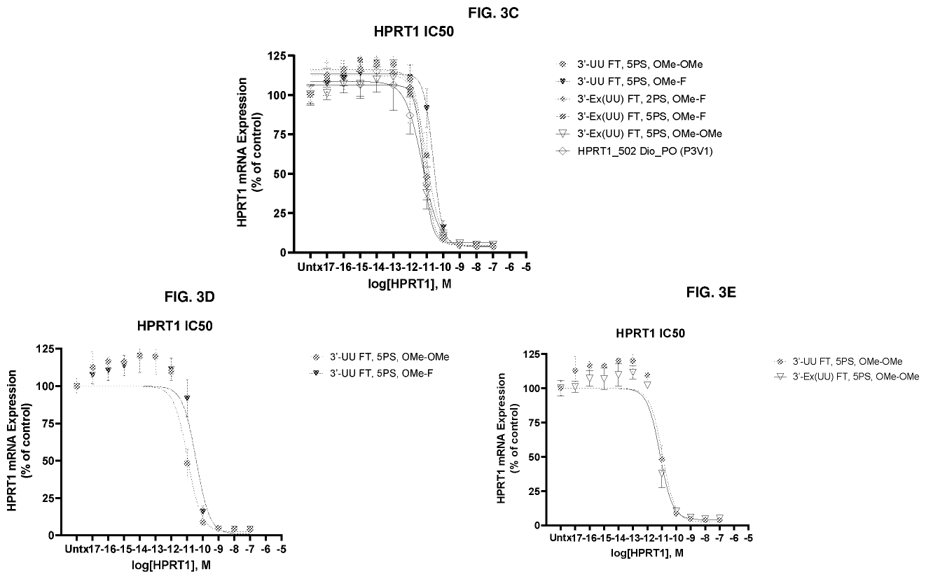

The results are shown in FIGS. 3A (3 nmol) and 3B (1 nmol). These results demonstrate that including fixed nucleobase sequences effectively silence HPRT1 mRNA in vivo.

In a separate experiment, the siRNA molecules described in Table 5 were tested at various doses in vitro using Hela cells and a 10 pt series: 100 nM and 10-fold down to 0.00000001 nM and mock treated. The cells were reverse transfected using 0.1% RNAiMAX, 0.5% FBS, 0.5% Penicillin-Streptomycin and their IC50 was calculated. The results are shown in FIGS. 3C (comparing all siRNA molecules), 3D (comparing Formula F1, containing a terminal 2′-methoxy nucleosides, with Formula F6, containing a terminal 2′-fluoro nucleoside) and 3E (comparing Formula F1 to Formula F5, in which modified internucleoside linkages of Formula E6a have been added). The IC50 values are also reported in Table 6, below.

TABLE 6

| siRNA ID | IC50 (pM) |
|---|---|
| 11 | 7.53 |
| 12 | 25.0 |
| 13 | 6.93 |
| 14 | 9.73 |
| 15 | 6.33 |
| 16 | 5.47 |

### Example 4. Evaluation of all Possible Fixed Trinucleotide Sequences at the 3′ End of the Antisense Strand of a ds-siRNA Molecule

In this experiment, the effect of all possible trinucleotide sequences (i.e., the 3′ sequences in Table 1a) was examined. A ds-siRNA molecule targeting HPRT1 having a 21 mer antisense strand and a 16 mer sense strand was utilized in this example. The three terminal nucleobases at the 3′ end of the antisense strand were fixed as the sequences in Table 1a, independent of the target mRNA transcript. These fixed nucleobase regions were chemically modified as shown in Formula F1.

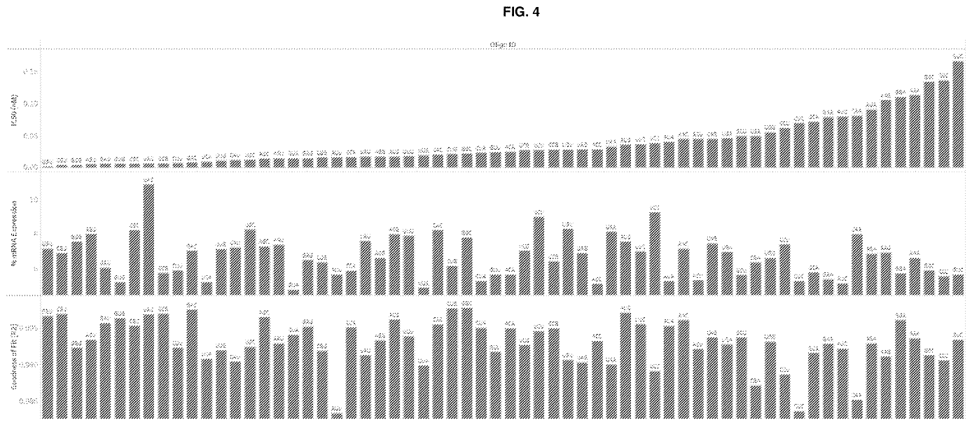

Hela cells were transfected with the siRNA molecules at doses ranging from 0.00000001 nM to 100 nM. The IC50 value was then calculated for each condition tested, with the results shown in FIG. 4. The lowest IC50 value was observed for the GGU sequence (1.9 μM) while the highest IC50 value was observed for the GUC sequence (165 μM). The IC50 values are also reported in Table 7, below.

TABLE 7

| | Compound | IC50 |
|---|---|---|
| Rank | Tail 5′-3′ | (pM) |
| 1 | GGU | 1.90 |
| 2 | CGU | 4.14 |
| 3 | GGG | 4.20 |
| 4 | AGU | 5.46 |
| 5 | GAU | 5.70 |
| 6 | GUG | 5.72 |
| 7 | CGC | 6.26 |
| 8 | UAC | 6.35 |
| 9 | GCG | 6.73 |
| 10 | CUU | 7.31 |
| 11 | GAC | 7.68 |
| 12 | UCA | 9.13 |
| 13 | UUG | 11.40 |
| 14 | CAU | 12.30 |
| 15 | UGC | 12.50 |
| 16 | AGC | 13.70 |
| 17 | AAU | 14.60 |
| 18 | GAG | 14.80 |
| 19 | GUA | 14.80 |
| 20 | CGG | 15.90 |
| 21 | AUU | 16.00 |
| 22 | CCA | 16.70 |
| 23 | AGG | 17.30 |
| 24 | UAU | 17.30 |
| 25 | ACG | 17.70 |
| 26 | UUU | 18.50 |
| 27 | UUA | 20.00 |
| 28 | CAC | 20.70 |
| 29 | CUG | 21.40 |
| 30 | GGC | 22.00 |
| 31 | CUA | 23.40 |
| 32 | ACA | 24.50 |
| 33 | UCG | 27.20 |
| 34 | UCU | 27.60 |
| 35 | CCG | 27.90 |
| 36 | UAG | 28.80 |
| 37 | ACC | 29.10 |
| 38 | UGU | 31.60 |
| 39 | GUU | 33.90 |
| 40 | UAA | 34.00 |
| 41 | AUG | 35.60 |
| 42 | UUC | 37.90 |
| 43 | UCC | 38.40 |
| 44 | AUA | 40.30 |
| 45 | AAC | 44.70 |
| 46 | UGA | 45.30 |
| 47 | CAG | 45.30 |
| 48 | GCU | 48.80 |
| 49 | CGA | 49.20 |
| 50 | ACU | 53.20 |
| 51 | UGG | 55.00 |
| 52 | GCA | 77.90 |
| 53 | GAA | 78.80 |
| 54 | AUC | 79.20 |
| 55 | CUC | 79.30 |
| 56 | CAA | 80.40 |
| 57 | CCU | 88.70 |
| 58 | AGA | 90.30 |
| 59 | AAG | 105.00 |
| 60 | GGA | 110.00 |
| 61 | AAA | 113.00 |
| 62 | CCC | 137.00 |
| 63 | GCC | 158.00 |
| 64 | GUC | 166.00 |

This example demonstrates that the identity of the nucleobase sequence at the 3′ end of the antisense strand influences the silencing activity of an siRNA molecule.

### Example 5. Method of Delivering a ds-siRNA Molecule to the Central Nervous System of a Patient

A subject, such as a human subject, diagnosed with a disease is treated with a dose and frequency determined by a practitioner (e.g., three times daily, twice daily, once daily, once weekly, once monthly) by administering the siRNA molecule of the disclosure of a pharmaceutical composition containing the same. Dosage and frequency are determined based on the subject's height, weight, age, sex, and other disorders.

A siRNA molecule (e.g., a branched siRNA molecule) having a pattern of chemical modifications disclosed herein is selected by the practitioner for compatibility with the disease and subject. Single- or double-stranded branched siRNA are available for selection. The siRNA chosen has an antisense strand, and in the case of double-stranded siRNA, a sense strand with a sequence and RNA modifications (e.g., natural and non-natural internucleoside linkages, modified sugars, and 5′-phosphorus stabilizing moieties) best suited to the patient and the disease being targeted. For example, the antisense strand may have any one of the antisense strand modification patterns disclosed herein, such as, e.g., Antisense Pattern 1: A-S-B-S-A-O-B-O-B-O-B-O-A-O-B-O-A-O-B-O-A-O-B-O-A-O-B-O-A-O-B-S-A-S-A-S-A-S-A-S-A (Formula A1); Antisense Pattern 2: A-S-B-S-A-O-B-O-A-O-B-O-A-O-B-O-A-O-B-O-A-O-B-O-A-O-B-O-A-O-B-S-A-S-B-S-A-S-A-S-A (Formula A2); or Antisense Pattern 3: A-S-B-S-A-O-A-O-A-O-B-O-A-O-A-O-A-O-A-O-A-O-A-O-A-O-B-O-A-O-B-S-A-S-A-S-A-S-B-S-A (Formula A3). In the case of a ds-siRNA, Antisense Pattern 1 may have a fully or partially complementary sense strand having any one of the patterns of chemical modifications of Sense Pattern 1: A-S-A-S-A-O-B-O-A-O-B-O-A-O-B-O-A-O-B-O-A-O-A-O-A-O-B-S-A-S-A (Formula S1); Sense Pattern 2: A-S-A-S-A-O-B-O-A-O-B-O-A-O-B-O-A-O-B-O-A-O-A-O-A-O-B-O-A-O-A (Formula S2); Sense Pattern 3: A-S-A-S-A-O-B-O-A-O-B-O-A-O-B-O-A-O-B-O-A-O-A-O-A-O-B-S-A-S-B (Formula S3); or Sense Pattern 4: A-S-A-S-A-O-B-O-A-O-B-O-A-O-B-O-A-O-B-O-A-O-A-O-A-O-B-O-A-O-B (Formula S4). In the case of a ds-siRNA having an Antisense Pattern 2, the sense strand may have any one of the patterns of chemical modifications of Sense Pattern 5: A-S-A-S-A-O-B-O-A-O-B-O-A-O-B-O-A-O-B-O-A-O-B-O-A-O-B-S-A-S-A (Formula S5); Sense Pattern 6: A-S-A-S-A-O-B-O-A-O-B-O-A-O-B-O-A-O-B-O-A-O-B-O-A-O-B-O-A-O-A (Formula S6); Sense Pattern 7: A-S-A-S-A-O-B-O-A-O-B-O-A-O-B-O-A-O-B-O-A-O-B-O-A-O-B-S-A-S-B (Formula S7); or Sense Pattern 8: A-S-A-S-A-O-B-O-A-O-B-O-A-O-B-O-A-O-B-O-A-O-B-O-A-O-B-O-A-O-B (Formula S8). In the case of a ds-siRNA having an Antisense Pattern 3, the sense strand may have a sense strand having a pattern of modifications of Sense Pattern 9: A-S-A-S-A-O-A-O-A-O-B-O-B-O-B-O-A-O-B-O-A-O-A-O-A-O-A-S-A-S-A (Formula S9); wherein A and B are different nucleosides (e.g., A is a 2-O-methyl ribonucleoside; B is a 2′-fluoro ribonucleoside), T is phosphorothioate, P is a phosphodiester, and PSM is a 5′-phosphorus stabilizing moiety (e.g., 5′-vinylphosphonate).

The siRNA is delivered by the route best suited the patient and condition (e.g., intrathecally, intracerebroventricularly, or intrastriatally), at a rate tolerable to the patient until the subject has reached a maximum tolerated dose, or until the symptoms of the disease are ameliorated satisfactorily.

### Example 6. Optimizing siRNA Molecules

It is contemplated that for any small interfering RNA (siRNA) agent disclosed herein, modifications to the siRNA may further optimize the molecule's efficacy or biophysical properties (e.g., increasing serum stability or circulating half-life, increasing thermal stability, enhancing transmembrane delivery, and/or targeting to a particular location or cell type). Such optimization could be achieved by systematically either adding or removing linked nucleosides to generate longer or shorter sequences. Further siRNA optimization could include the incorporation of, for example, one or more alternative nucleosides, alternative 2′ sugar moieties, and/or alternative internucleoside linkages. Further still, such optimized siRNA molecules may include the introduction of hydrophobic and/or stabilizing moieties at the 5′ and/or 3′ ends.

siRNA Optimization with Alternative Nucleosides

Optimization of the siRNA molecules of the disclosure may include one or more of the following nucleoside modifications: 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH3) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine, and/or 3-deazaguanine and 3-deazaadenine. The siRNA molecules may also include nucleobases in which the purine or pyrimidine base is replaced with other heterocycles, for example 7-deaza-adenine, 7-deazaguanosine, 2-aminopyridine, and/or 2-pyridone. Further optimization of the siRNA molecules of the disclosure may include nucleobases disclosed in U.S. Pat. No. 3,687,808; Kroschwitz, J. I., ed. The Concise Encyclopedia of Polymer Science and Engineering, New York, John Wiley & Sons, 1990, pp. 858-859; Englisch et al., Angewandte Chemie, International Edition 30:613, 1991; and Sanghvi, Y. S., Chapter 16, Antisense Research and Applications, CRC Press, Gait, M. J. ed., 1993, pp. 289-302.

siRNA Optimization with Alternative Sugar Modifications

Optimization of the siRNA molecules of the disclosure may include one or more of the following 2′ sugar modifications: 2′-O-methyl(2′-O-Me), 2′-methoxyethoxy (2′-O-CH₂CH₂OCH₃, also known as 2′-O-(2-methoxyethyl) or 2′-MOE), 2′-dimethylaminooxyethoxy, i.e., a O(CH₂)₂ON(CH₃)₂ group, also known as 2′-DMAOE, and/or 2′-dimethylaminoethoxyethoxy (also known in the art as 2′-O-dimethylamino-ethoxy-ethyl or 2′-DMAEOE), i.e., 2′-O-CH₂OCH₂N(CH₃)₂. Other possible 2′-modifications that can optimize the siRNA molecules of the disclosure include all possible orientations of OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C1 to C10 alkyl or C2 to C10 alkenyl and alkynyl. Other potential sugar substituent groups include, e.g., aminopropoxy (—OCH₂CH₂CH₂NH₂), allyl (—CH₂—CH═CH₂), —O-allyl (—O—CH₂—CH═CH₂) and fluoro (F). 2′-sugar substituent groups may be in the arabino (up) position or ribo (down) position. In some embodiments, the 2′-arabino modification is 2′-F. Similar modifications may also be made at other positions on the siRNA molecule, particularly the 3′ position of the sugar on the 3′ terminal nucleoside or in 2′-5′ linked oligonucleotides and the 5′ position of 5′ terminal nucleotide. Oligonucleotides may also have sugar mimetics such as cyclobutyl moieties in place of the pentofuranosyl sugar.

siRNA Optimization with Alternative Internucleoside Linkages

Optimization of the siRNA molecules of the disclosure may include one or more of the following internucleoside modifications: phosphorothioates, phosphorodithioates, phosphotriesters, aminoalkylphosphotriesters, methyl and other alkyl phosphonates including 3′-alkylene phosphonates, 5′-alkylene phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates, and boranophosphates having normal 3′-5′ linkages, 2′-5′ linked analogs of these, and those having inverted polarity wherein one or more internucleoside linkages is a 3′ to 3′, 5′ to 5′ or 2′ to 2′ linkage.

siRNA Optimization with Hydrophobic Moieties

Optimization of the siRNA molecules of the disclosure may include hydrophobic moieties covalently attached to the 5′ end or the 3′ end. Non-limiting examples of hydrophobic moieties suitable for use with the siRNA molecules of the disclosure may include cholesterol, vitamin D, tocopherol, phosphatidylcholine (PC), docohexaenoic acid, docosanoic acid, PC-docosanoic acid, eicosapentaenoic acid, lithocholic acid or any combination of the aforementioned hydrophobic moieties with PC.

siRNA Optimization with Stabilizing Moieties

Optimization of the siRNA molecules of the disclosure may include a 5′-phosphorous stabilizing moiety that protects the siRNA molecules from degradation. A 5′-phosphorus stabilizing moiety replaces the 5′-phosphate to prevent hydrolysis of the phosphate. Hydrolysis of the 5′-phosphate prevents binding to RISC, a necessary step in gene silencing. Any replacement for phosphate that does not impede binding to RISC is contemplated in this disclosure. In some embodiments, the replacement for the 5′-phosphate is also stable to in vivo hydrolysis. Each siRNA strand may independently and optionally employ any suitable 5′-phosphorus stabilizing moiety. Non-limiting examples of 5′ stabilizing moieties suitable for use with the siRNA molecules of the disclosure may include those demonstrated by Formulas IX-XVI above.

siRNA Optimization with Branched siRNA

Optimization of the siRNA molecules of the disclosure may include the incorporation of branching patterns, such as, for example, di-branched, tri-branched, or tetra-branched siRNAs connected by way of a linker. Each main branch may be further branched to allow for 2, 3, 4, 5, 6, 7, or 8 separate RNA single- or double-strands. The branch points on the linker may stem from the same atom, or separate atoms along the linker. Some exemplary embodiments are listed in Table 2, above.

The siRNA composition of the disclosure may be optimized to be in the form of: di-branched siRNA molecules, as represented by any one of Formulas XVII-XIX; tri-branched siRNA molecules, as represented by any one of Formulas XX-XXIII; and/or tetra-branched siRNA molecules, as represented by any one of Formulas XXIV-XXVIII, wherein each RNA, independently, is an siRNA molecule, L is a linker, and each X, independently, represents a branch point moiety (e.g., phosphoroamidite, tosylated solketal, 1,3-diaminopropanol, pentaerythritol, or any one of the branch point moieties described in U.S. Pat. No. 10,478,503).

### Example 7. Preparation and Administrating siRNA Molecules

The siRNA molecules in the present disclosure may be formulated into a pharmaceutical composition for administration to a subject in a biologically compatible form suitable for administration in vivo. For example, the siRNA molecules of the disclosure may be administered in a suitable diluent, carrier, or excipient, and may further contain a preservative, e.g., to prevent the growth of microorganisms. Conventional procedures and ingredients for the selection and preparation of suitable formulations are described, for example, in Remington, J. P. The Science and Practice of Pharmacy, Easton, PA. Mack Publishers, 2012, 22^{nd} ed. And in The United States Pharmacopeial Convention, The National Formulary, United States Pharmacopeial, 2015, USP 38 NF 33).

The method of the disclosure contemplates any route of administration to the subject's CNS that is tolerated by the siRNA compositions of the disclosure. Non-limiting examples of siRNA injections into the CNS include intrathecally, intracerebroventricularly, or intra-cisterna magna injection by catheterization. A physician having ordinary skill in the art can readily determine an effective route of administration.

### Example 8. Methods for the Treatment of a Subject in Need of Gene Silencing

A subject in need of gene silencing is treated with a dosage of the siRNA molecule or siRNA composition of the disclosure, formulated as a salt, at frequency determined by a practitioner. A physician having ordinary skill in the art can readily determine an effective amount of the siRNA molecule for administration to a mammalian subject (e.g., a human) in need thereof. For example, a physician could start prescribing doses of one of the siRNA molecules of the disclosure at levels lower than that required to achieve the desired therapeutic effect and gradually increase the dosage until the desired effect is achieved. Alternatively, a physician may begin a treatment regimen by administering one of the siRNA molecules of the disclosure at a high dose and subsequently administer progressively lower doses until a minimum dose that produces a therapeutic effect (e.g., a reduction in expression of a target mRNA or suitable biomarker) is achieved. In general, a suitable daily dose of one of one of the siRNA molecules of the disclosure will be an amount which is the lowest dose effective to produce a therapeutic effect. The ss- or ds-siRNA molecules of the disclosure may be administered by injection, e.g., intrathecally, intracerebroventricularly, or by intra-cisterna magna injection via catheterization. A daily dose of a therapeutic composition of one of the siRNA molecules of the disclosure may be administered as a single dose or as two, three, four, five, six or more doses administered separately at appropriate intervals throughout the day, week, month, or year, optionally, in unit dosage forms. While it is possible for any of the siRNA molecules of the disclosure to be administered alone, it may also be administered as a pharmaceutical formulation in combination with excipients, carriers, and optionally, additional therapeutic agents. Dosage and frequency are determined based on the subject's height, weight, age, sex, and other disorders.

The siRNA molecule(s) of the disclosure is selected by the practitioner for compatibility with the subject. Single- or double-stranded siRNA molecules (e.g., non-branched siRNA, di-branched siRNA, tri-branched siRNA, tetra-branched siRNA, covalently linked siRNA) are available for selection. The siRNA molecule chosen has an antisense strand and may have a sense strand with a sequence and RNA modifications (e.g., natural and non-natural internucleoside linkages, modified sugars, 5′-phosphorus stabilizing moieties, hydrophobic moieties, and/or branching structures) best suited to the patient.

The siRNA molecule is delivered by the route best suited the patient (e.g., intrathecally, intracerebroventricularly, or by intra-cisterna magna injection via catheterization) and condition at a rate tolerable to the patient until the subject has reached a maximum tolerated dose, or until symptoms are ameliorated satisfactorily.

### OTHER EMBODIMENTS

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

1. A small interfering RNA (siRNA) molecule comprising an antisense strand and a sense strand having complementarity to a portion of the antisense strand; wherein:
(i) the antisense strand comprises, in the 5′-to-3′ direction, a first region of linked nucleotides and a second region of linked nucleotides;
(ii) the first region has complementarity sufficient to hybridize to a portion of a target mRNA transcript;
(iii) the second region comprises an overhang that extends beyond the sense strand; and
(iv) the second region has one or more nucleotide mismatches relative to the target mRNA transcript.

2. An siRNA molecule comprising an antisense strand and a sense strand having complementarity to a portion of the antisense strand; wherein:
(i) the antisense strand comprises, in the 5′-to-3′ direction, a first region of linked nucleotides and a second region of linked nucleotides;
(ii) the first region has complementarity sufficient to hybridize to a portion of a target mRNA transcript;
(iii) the second region comprises an overhang that extends beyond the sense strand; and
(iv) the second region has a fixed nucleobase sequence that is independent of the nucleobase sequence of the target mRNA transcript.

3. The siRNA molecule of claim 1 or 2, wherein upon introducing the siRNA molecule into an RNA-induced silencing complex (RISC) and exposing the RISC to the target mRNA transcript, the target mRNA transcript is cleaved, thereby forming cleavage products, and the cleavage products dissociate from the RISC with an increased off-rate relative to a RISC formed from a corresponding siRNA comprising an antisense strand that is fully complementary to the target mRNA transcript.

4. The siRNA molecule of any one of claims 1-3, wherein the nucleobase sequence of the second region imparts the antisense strand with an increased binding affinity for an endogenous Argonaute (AGO) protein relative to a corresponding antisense strand that is fully complementary to the target mRNA.

5. The siRNA molecule of any one of claims 1-4, wherein the nucleobase sequence of the second region improves the half-life of an endogenous complex comprising the antisense strand and an AGO protein relative to a corresponding endogenous complex comprising a corresponding antisense strand that is fully complementary to the target mRNA.

6. The siRNA molecule of any one of claims 1-5, wherein the second region is from 1 to 10 nucleotides in length, optionally wherein the second region is from 1 to 6 nucleotides in length.

7. The siRNA molecule of claim 6, wherein the second region is 1 nucleotide in length.

8. The siRNA molecule of claim 6, wherein the second region is 2 nucleotides in length.

9. The siRNA molecule of claim 6, wherein the second region is 3 or 4 nucleotides in length.

10. The siRNA molecule of any one of claims 1-9, wherein the second region comprises at least one uridine nucleotide.

11. The siRNA molecule of claim 10, wherein the second region comprises two uridine nucleotides.

12. The siRNA molecule of any one of claims 1-11, wherein the second region comprises at least one modified internucleoside linkage.

13. The siRNA molecule of claim 12, wherein the second region comprises at least one modified internucleoside linkage of Formula E1: 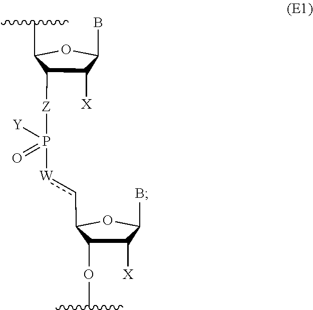
wherein:
each B is, independently, a base pairing moiety;
W is selected from the group consisting of O, S, B, BR², N, NR², OCH₂, OCH, CH₂, and CH;
each X is, independently, selected from the group consisting of halo, hydroxy, and C₁₋₆ alkoxy;
Y is selected from the group consisting of O⁻, OH, OR, NH⁻, NH₂, S⁻, and SH;
Z is selected from the group consisting of O, S, BR², NR², and CH₂;
R is a protecting group;
each R² is, independently, H or optionally substituted C₁-C₆ alkyl; and [symbol]
is an optional double bond.

14. The siRNA molecule of claim 13, wherein when Y is O⁻, either Z or W is not O.

15. The siRNA molecule of claim 13, wherein Z is CH₂ and W is CH₂.

16. The siRNA molecule of claim 12, wherein the modified internucleoside linkage of Formula E1 is a modified internucleoside linkage of Formula E2:

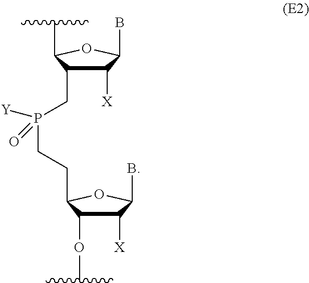

17. The siRNA molecule of claim 13, wherein Z is CH₂ and W is O.

18. The siRNA molecule of claim 12, wherein the modified internucleoside linkage of Formula E1 is a modified internucleoside linkage of Formula E3:

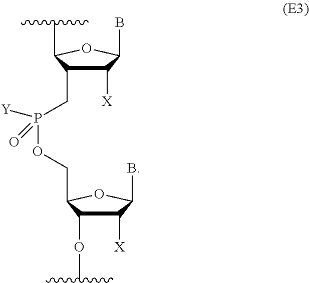

19. The siRNA molecule of claim 13, wherein Z is O and W is CH₂.

20. The siRNA molecule of claim 12, wherein the modified internucleoside linkage of Formula E1 is a modified internucleoside linkage of Formula E4:

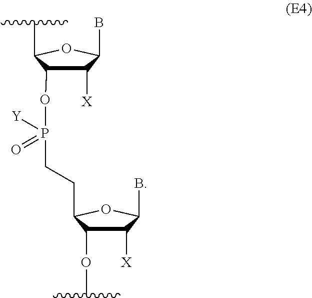

21. The siRNA molecule of claim 13, wherein Z is O and W is CH.

22. The siRNA molecule of claim 12, wherein the modified internucleoside linkage of Formula E1 is a modified internucleoside linkage of Formula E5:

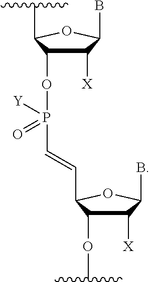

23. The siRNA molecule of claim 13, wherein Z is O and W is OCH₂.

24. The siRNA molecule of claim 12, wherein the modified internucleoside linkage of Formula E1 is a modified internucleoside linkage of Formula E6: 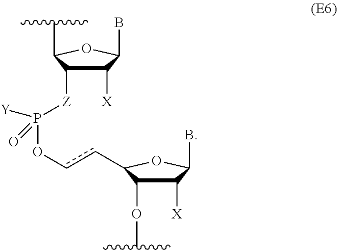
wherein
each X is, independently, selected from the group consisting of fluoro, hydroxy, and C₁₋₆ alkoxy;
Y is selected from the group consisting of O⁻, OH, OR, S⁻, SH, and SR;
Z is selected from the group consisting of O and CH₂; and [symbol]
is an optional double bond,

25. The siRNA molecule of claim 24 wherein the modified internucleoside linkage of Formula E1 is a modified internucleoside linkage of Formula E6a:

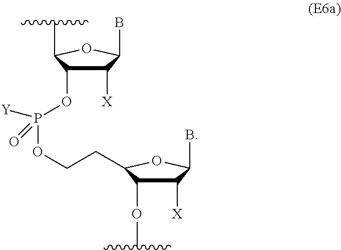

26. The siRNA molecule of claim 13, wherein Z is CH₂ and W is CH.

27. The siRNA molecule of claim 12, wherein the modified internucleoside linkage of Formula E1 is a modified internucleoside linkage of Formula E7:

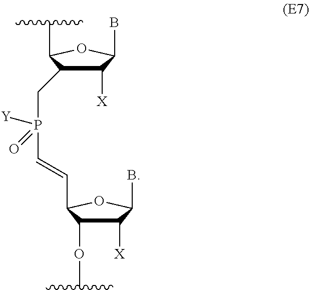

28. The siRNA molecule of any one of claims 1-27, wherein the second region comprises at least one phosphorothioate internucleoside linkage.

29. The siRNA molecule of any one of claims 1-28, wherein the second region comprises at least one nucleotide comprising a modified ribose.

30. The siRNA molecule of any claim 29, wherein the second region comprises at least one 2′-methoxy nucleotide.

31. The siRNA molecule of claim 29 or 30, wherein the second region comprises at least one 2′-fluoro nucleotide.

32. The siRNA molecule of any one of claims 1-31, wherein the second region has any one of the following sequences, in the 5′ to 3′ direction: 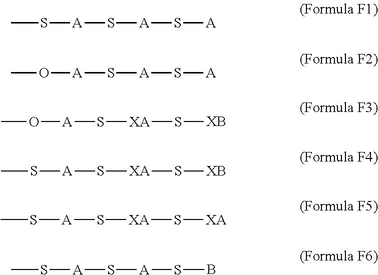
wherein
each S is a phosphorothioate internucleoside linkage;
each O is a phosphodiester internucleoside linkage;
each A is a 2′-methoxy ribonucleoside;
each B is a 2′-fluoro ribonucleoside;
each XA is a 2′-methoxy nucleotide of Formula E6a; and
each XB is a 2′-fluoro nucleotide of Formula E6a.

33. The siRNA molecule of claim 32, wherein the second region has any one of the following sequences, in the 5′ to 3′ direction: 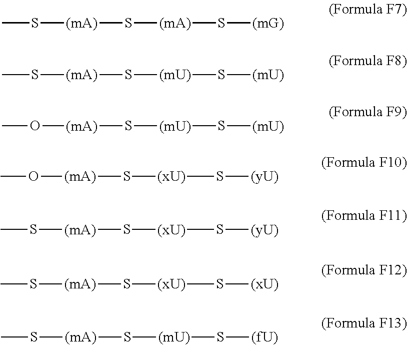
wherein
each S is a phosphorothioate internucleoside linkage,
each O is a phosphodiester internucleoside linkage,
each mA is a 2′-methoxyadenosine,
each mG is a 2′-methoxy guanidine,
each mU is a 2′-methoxyuridine,
each xU is a 2′methoxyuridine of Formula E6a, and
each yU is a 2′-fluorouridine of Formula E6a.

34. The siRNA molecule of any one of claims 1-33, wherein the antisense strand comprises a structure represented by Formula I, wherein Formula I is, in the 5′-to-3′ direction: 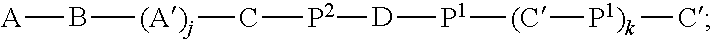
wherein A is represented by the formula C-P¹-D-P¹;
each A′ is represented by the formula C-P²-D-P²;
B is represented by the formula C-P²-D-P²-D-P²-D-P²;
each C is a 2′-O-methyl(2′-O-Me) ribonucleoside;
each C′, independently, is a 2′-O-Me ribonucleoside or a 2′-fluoro (2′-F) ribonucleoside;
each D is a 2′-F ribonucleoside;
each P¹ is a phosphorothioate internucleoside linkage;
each P² is a phosphodiester internucleoside linkage;
j is an integer from 1 to 7; and
k is an integer from 1 to 7.

35. The siRNA molecule of claim 34, wherein the antisense strand comprises a structure represented by Formula A1, wherein Formula A1 is, in the 5′-to-3′ direction: 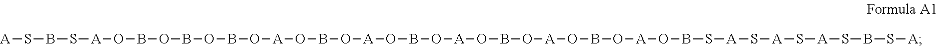
wherein A represents a 2′-O-Me ribonucleoside, B represents a 2′-F ribonucleoside, O represents a phosphodiester internucleoside linkage, and S represents a phosphorothioate internucleoside linkage.

36. The siRNA molecule of any one of claims 1-33, wherein the antisense strand comprises a structure represented by Formula II, wherein Formula II is, in the 5′-to-3′ direction: 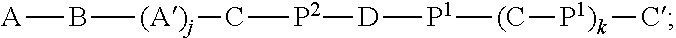
wherein A is represented by the formula C-P¹-D-P¹;
each A′ is represented by the formula C-P²-D-P²;
B is represented by the formula C-P²-D-P²-D-P²-D-P²;
each C is a 2′-O-methyl(2′-O-Me) ribonucleoside;
each C′, independently, is a 2′-O-Me ribonucleoside or a 2′-fluoro (2′-F) ribonucleoside;
each D is a 2′-F ribonucleoside;
each P¹ is a phosphorothioate internucleoside linkage;
each P² is a phosphodiester internucleoside linkage;
j is an integer from 1 to 7; and
k is an integer from 1 to 7.

37. The siRNA molecule of claim 36, wherein the antisense strand comprises a structure represented by Formula A2, wherein Formula A2 is, in the 5′-to-3′ direction: 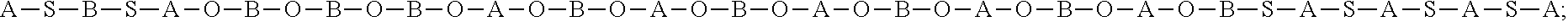
wherein A represents a 2′-O-Me ribonucleoside, B represents a 2′-F ribonucleoside, O represents a phosphodiester internucleoside linkage, and S represents a phosphorothioate internucleoside linkage.

38. The siRNA molecule of any one of claims 1-37, wherein the sense strand comprises a structure represented by Formula III, wherein Formula III is, in the 5′-to-3′ direction: 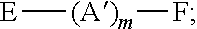
wherein E is represented by the formula (C-P¹)₂;
F is represented by the formula (C-P²)₃-D-P¹-C-P¹-C, (C-P²)₃-D-P²-C-P²-C, (C-P²)₃-D-P¹-C-P¹-D, or (C-P²)₃-D-P²-C-P²-D;
A′, C, D, P¹, and P² are as defined in Formula II; and
m is an integer from 1 to 7.

39. The siRNA molecule of claim 38, wherein the sense strand comprises a structure represented by Formula S1, wherein Formula S1 is, in the 5′-to-3′ direction: 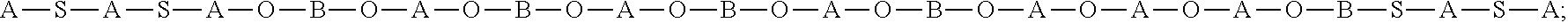
wherein A represents a 2′-O-Me ribonucleoside, B represents a 2′-F ribonucleoside, O represents a phosphodiester internucleoside linkage, and S represents a phosphorothioate internucleoside linkage.

40. The siRNA molecule of claim 38, wherein the sense strand comprises a structure represented by Formula S2, wherein Formula S2 is, in the 5′-to-3′ direction: 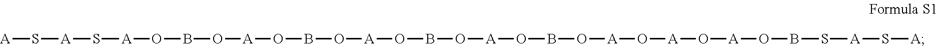
wherein A represents a 2′-O-Me ribonucleoside, B represents a 2′-F ribonucleoside, O represents a phosphodiester internucleoside linkage, and S represents a phosphorothioate internucleoside linkage.

41. The siRNA molecule of claim 38, wherein the sense strand comprises a structure represented by Formula S3, wherein Formula S3 is, in the 5′-to-3′ direction: 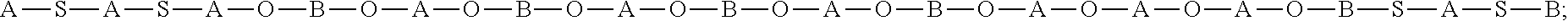
wherein A represents a 2′-O-Me ribonucleoside, B represents a 2′-F ribonucleoside, O represents a phosphodiester internucleoside linkage, and S represents a phosphorothioate internucleoside linkage.

42. The siRNA molecule of claim 38, wherein the sense strand comprises a structure represented by Formula S4, wherein Formula S4 is, in the 5′-to-3′ direction: 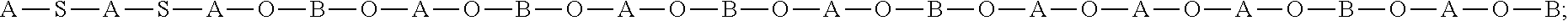
wherein A represents a 2′-O-Me ribonucleoside, B represents a 2′-F ribonucleoside, O represents a phosphodiester internucleoside linkage, and S represents a phosphorothioate internucleoside linkage.

43. The siRNA molecule of any one of claims 1-33 and 38-42, wherein the antisense strand comprises a structure represented by Formula IV, wherein Formula IV is, in the 5′-to-3′ direction: 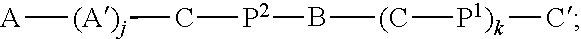
wherein A is represented by the formula C-P¹-D-P¹;
each A′ is represented by the formula C-P²-D-P²;
B is represented by the formula D-P¹-C-P¹-D-P¹;
each C is a 2′-O-Me ribonucleoside;
each C′, independently, is a 2′-O-Me ribonucleoside or a 2′-F ribonucleoside;
each D is a 2′-F ribonucleoside;
each P¹ is a phosphorothioate internucleoside linkage;
each P² is a phosphodiester internucleoside linkage;
j is an integer from 1 to 7; and
k is an integer from 1 to 7.

44. The siRNA molecule of claim 43, wherein the antisense strand comprises a structure represented by Formula A3, wherein Formula A3 is, in the 5′-to-3′ direction: 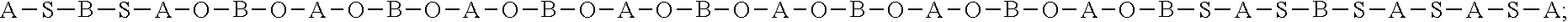
wherein A represents a 2′-O-Me ribonucleoside, B represents a 2′-F ribonucleoside, O represents a phosphodiester internucleoside linkage, and S represents a phosphorothioate internucleoside linkage.

45. The siRNA molecule of any one of claims 1-37, 43, and 44, wherein the sense strand comprises a structure represented by Formula V, wherein Formula V is, in the 5′-to-3′ direction: 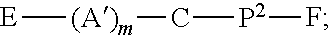
wherein E is represented by the formula (C-P¹)₂;
F is represented by the formula D-P¹-C-P¹-C, D-P²-C-P²-C, D-P¹-C-P¹-D, or D-P²-C-P²-D;
A′, C, D, P¹ and P² are as defined in Formula IV; and
m is an integer from 1 to 7.

46. The siRNA molecule of claim 45, wherein the sense strand comprises a structure represented by Formula S5, wherein Formula S5 is, in the 5′-to-3′ direction: 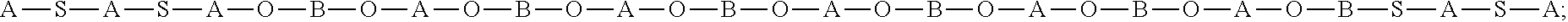
wherein A represents a 2′-O-Me ribonucleoside, B represents a 2′-F ribonucleoside, O represents a phosphodiester internucleoside linkage, and S represents a phosphorothioate internucleoside linkage.

47. The siRNA molecule of claim 45, wherein the sense strand comprises a structure represented by Formula S6, wherein Formula S6 is, in the 5′-to-3′ direction: 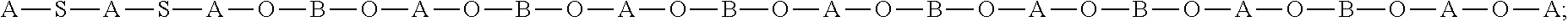
wherein A represents a 2′-O-Me ribonucleoside, B represents a 2′-F ribonucleoside, O represents a phosphodiester internucleoside linkage, and S represents a phosphorothioate internucleoside linkage.

48. The siRNA molecule of claim 45, wherein the sense strand comprises a structure represented by Formula S7, wherein Formula S7 is, in the 5′-to-3′ direction: 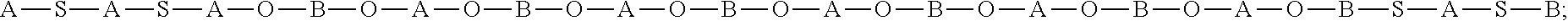
wherein A represents a 2′-O-Me ribonucleoside, B represents a 2′-F ribonucleoside, O represents a phosphodiester internucleoside linkage, and S represents a phosphorothioate internucleoside linkage.

49. The siRNA molecule of claim 45, wherein the sense strand comprises a structure represented by Formula S8, wherein Formula S8 is, in the 5′-to-3′ direction: 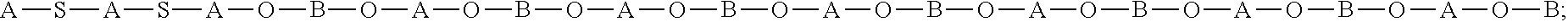
wherein A represents a 2′-O-Me ribonucleoside, B represents a 2′-F ribonucleoside, O represents a phosphodiester internucleoside linkage, and S represents a phosphorothioate internucleoside linkage.

50. The siRNA molecule of any one of claims 1-33, 38-42, and 45-49, wherein the antisense strand comprises a structure represented by Formula VI, wherein Formula VI is, in the 5′-to-3′ direction: 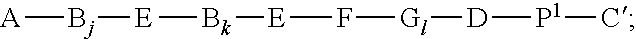
wherein A is represented by the formula C-P¹-D-P¹;
each B is represented by the formula C-P²;
each C is a 2′-O-Me ribonucleoside;
each C′, independently, is a 2′-O-Me ribonucleoside or a 2′-F ribonucleoside;
each D is a 2′-F ribonucleoside;
each E is represented by the formula D-P²-C-P²;
F is represented by the formula D-P¹-C-P¹;
each G is represented by the formula C-P¹;
each P¹ is a phosphorothioate internucleoside linkage;
each P² is a phosphodiester internucleoside linkage;
j is an integer from 1 to 7;
k is an integer from 1 to 7; and
l is an integer from 1 to 7.

51. The siRNA molecule of claim 50, wherein the antisense strand comprises a structure represented by Formula A4, wherein Formula A4 is, in the 5′-to-3′ direction: 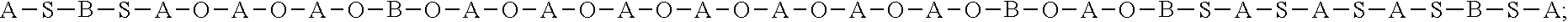
wherein A represents a 2′-O-Me ribonucleoside, B represents a 2′-F ribonucleoside, O represents a phosphodiester internucleoside linkage, and S represents a phosphorothioate internucleoside linkage.

52. The siRNA molecule of any one of claims 1-37, 43, 44, 50, and 51, wherein the sense strand comprises a structure represented by Formula VII, wherein Formula VII is, in the 5′-to-3′ direction: 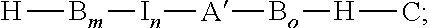
wherein A′ is represented by the formula C-P²-D-P²;
each H is represented by the formula (C-P¹)₂;
each I is represented by the formula (D-P²);
B, C, D, P¹ and P² are as defined in Formula VI;
m is an integer from 1 to 7;
n is an integer from 1 to 7; and
o is an integer from 1 to 7.

53. The siRNA molecule of claim 52, wherein the sense strand comprises a structure represented by Formula S9, wherein Formula S9 is, in the 5′-to-3′ direction: 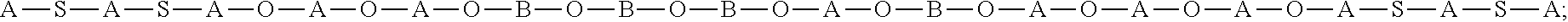
wherein A represents a 2′-O-Me ribonucleoside, B represents a 2′-F ribonucleoside, O represents a phosphodiester internucleoside linkage, and S represents a phosphorothioate internucleoside linkage.

54. The siRNA molecule of any one of claims 1-53, wherein the antisense strand further comprises a 5′ phosphorus stabilizing moiety at the 5′ end of the antisense strand.

55. The siRNA molecule of any one of claims 1-54, wherein the sense strand further comprises a 5′ phosphorus stabilizing moiety at the 5′ end of the sense strand.

56. The siRNA molecule of claim 54 or 55, wherein each 5′ phosphorus stabilizing moiety is, independently, represented by any one of Formulas IX-XVI: 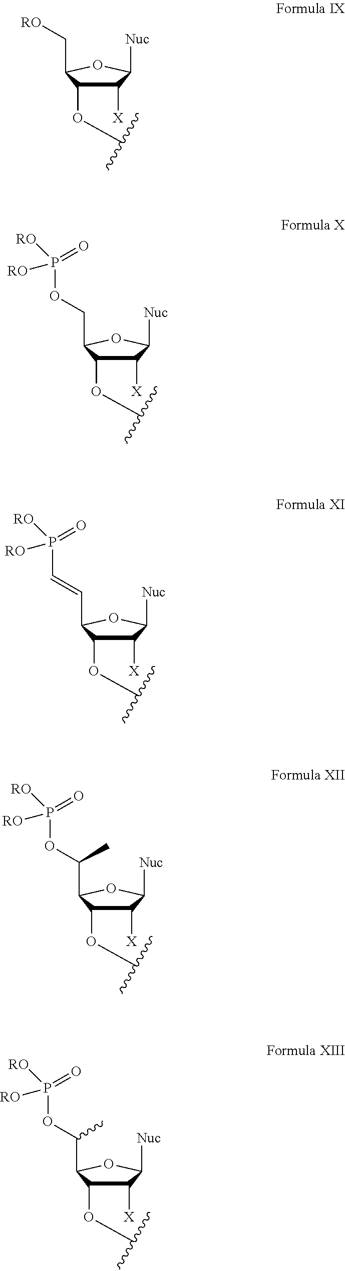 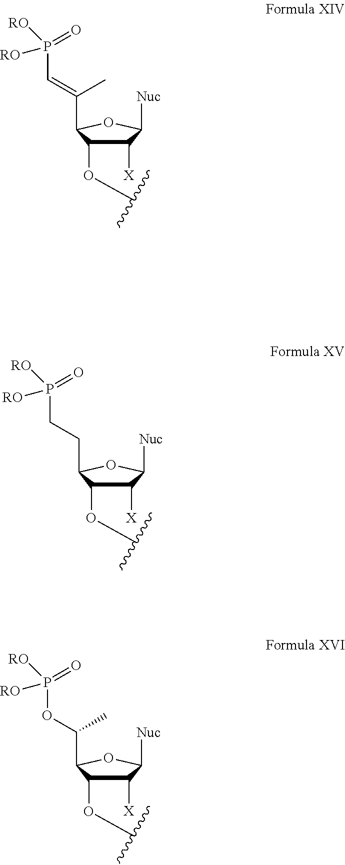
wherein Nuc represents a nucleobase selected from the group consisting of adenine, uracil, guanine, thymine, and cytosine, and R represents an optionally substituted alkyl, optionally substituted alkenyl, optionally substituted alkynyl, phenyl, benzyl, hydroxy, or hydrogen.

57. The siRNA molecule of claim 56, wherein the nucleobase is an adenine, uracil, guanine, thymine, or cytosine.

58. The siRNA molecule of any one of claims 54-57, wherein the 5′ phosphorus stabilizing moiety is (E)-vinylphosphonate represented by Formula XI.

59. The siRNA molecule of any one of claims 1-58, wherein the siRNA molecule further comprises a hydrophobic moiety at the 5′ or the 3′ end of the siRNA molecule.

60. The siRNA molecule of claim 59, wherein the hydrophobic moiety is selected from a group consisting of cholesterol, vitamin D, or tocopherol.

61. The siRNA molecule of any one of claims 1-60, wherein the length of the sense strand is between 10 and 30 nucleotides.

62. The siRNA molecule of claim 61, wherein the length of the sense strand is between 10 and 25 nucleotides.

63. The siRNA molecule of claim 62, wherein the length of the sense strand is between 12 and 25 nucleotides.

64. The siRNA molecule of claim 63, wherein the length of the sense strand is between 12 and 20 nucleotides.

65. The siRNA molecule of claim 64, wherein the length of the sense strand is between 12 and 19 nucleotides.

66. The siRNA molecule of claim 65, wherein the length of the sense strand is 15 nucleotides.

67. The siRNA molecule of claim 65, wherein the length of the sense strand is 16 nucleotides.

68. The siRNA molecule of claim 65, wherein the length of the sense strand is 18 nucleotides.

69. The siRNA molecule of any one of claims 1-68, wherein the length of the antisense strand is between 10 and 30 nucleotides.

70. The siRNA molecule of claim 69, wherein the length of the antisense strand is between 12 and 30 nucleotides.

71. The siRNA molecule of claim 70, wherein the length of the antisense strand is between 15 and 30 nucleotides.

72. The siRNA molecule of claim 71, wherein the length of the antisense strand is between 18 and 30 nucleotides.

73. The siRNA molecule of claim 72, wherein the length of the antisense strand is between 18 and 25 nucleotides.

74. The siRNA molecule of claim 73, wherein the length of the antisense strand is between 18 and 21 nucleotides.

75. The siRNA molecule of claim 74, wherein the length of the antisense strand is 18 nucleotides.

76. The siRNA molecule of claim 74, wherein the length of the antisense strand is 20 nucleotides.

77. The siRNA molecule of claim 74, wherein the length of the antisense strand is 21 nucleotides.

78. The siRNA molecule of any one of claims 1-77, wherein the siRNA molecule is a branched siRNA molecule.

79. The siRNA molecule of claim 78, wherein the branched siRNA molecule is di-branched, tri-branched, or tetra-branched.

80. The siRNA molecule of claim 79, wherein the siRNA molecule is a di-branched siRNA molecule, optionally wherein the di-branched siRNA molecule is represented by any one of Formulas XVII-XIX: 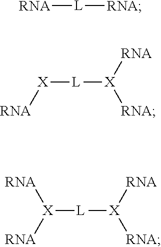
wherein each RNA is, independently, an siRNA molecule, L is a linker, and each X, independently, represents a branch point moiety.

81. The siRNA molecule of claim 79, wherein the siRNA molecule is a tri-branched siRNA molecule, optionally wherein the tri-branched siRNA molecule is represented by any one of Formulas XX-XXIII: 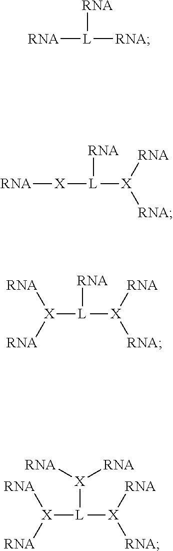
wherein each RNA is, independently, an siRNA molecule, L is a linker, and each X, independently, represents a branch point moiety.

82. The siRNA molecule of claim 79, wherein the siRNA molecule is a tetra-branched siRNA molecule, optionally wherein the tetra-branched siRNA molecule is represented by any one of Formulas XXIV-XXVIII: 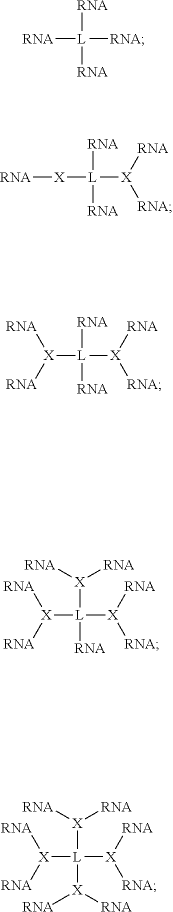
wherein each RNA is, independently, an siRNA molecule, L is a linker, and each X, independently, represents a branch point moiety.

83. The siRNA molecule of any one of claims 80-82, wherein the linker is selected from a group consisting of one or more contiguous subunits of an ethylene glycol, alkyl, carbohydrate, block copolymer, peptide, RNA, and DNA.

84. The siRNA molecule of claim 83, wherein the one or more contiguous subunits is 2 to 20 contiguous subunits.

85. A pharmaceutical composition comprising the siRNA molecule of any one of claims 1-84 and a pharmaceutically acceptable excipient, carrier, or diluent.

86. A method of delivering an siRNA molecule to the central nervous system (CNS) of a subject, the method comprising administering a therapeutically effective amount of the siRNA molecule of any one of claims 1-84 or the pharmaceutical composition of claim 85 to the subject.

87. A method of reducing expression of a target gene in a subject in need thereof, the method comprising administering a therapeutically effective amount of the siRNA molecule of any one of claims 1-84 or the pharmaceutical composition of claim 85 to the CNS of the subject.

88. The method of claim 86 or 87, wherein the siRNA molecule or the pharmaceutical composition is administered to the subject by way of intrastriatal, intracerebroventricular, or intrathecal injection.

89. The method of claim 87 or 88, wherein the target gene is an overactive disease driver gene.

90. The method of claim 87 or 88, wherein the target gene is a negative regulator of a gene with reduced expression that is associated with a disease state in the subject.

91. The method of claim 87 or 88, wherein the target gene is a positive regulator of a gene with increased expression that is associated with a disease state in a subject.

92. The method of claim 87 or 88, wherein the target gene is a splice isoform of the target gene, wherein the splice isoform reduces expression of the target gene.

93. The method of any one of claims 87-92, wherein the reduction in gene expression treats a disease state in the subject.

94. The method of any one of claims 86-93, wherein the subject is a human.

95. A kit comprising the multimeric oligonucleotide of any one of claims 1-84, or the pharmaceutical composition of claim 85, and a package insert, wherein the package insert instructs a user of the kit to perform the method of any one of claims 86-94.
